SEQUENCE LISTING (37 sequences)

SEQ ID NO: 1 - protein, 413 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 2 - DNA, 2033 bp, Homo sapiens
gcacataacgggcagaacgcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccaacagaacactaaacagactatttaacttgagggtaataaacttagaataaaattgtaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaagtctaaagtttacagtaaataaattgtttgaccatgcc

SEQ ID NO: 3 - protein, 407 aa, Homo sapiens
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS

SEQ ID NO: 4 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 5 - protein, 466 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLCRKQSSKHALGYTLHPPSQAVEGQHKDMVRIPVGSRETFYRISKTDGVCEWKFFSSMPRGSARITVSKDQSSCTTARVRSKSFLQVCCCVGPSTPSLDKNHQVPTIKVHTISLSENGEEV

SEQ ID NO: 6 - protein, 460 aa, Homo sapiens
MNTSAPPAVSPNITVLAPGKGPWQVAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENRARELAALQGSETPGKGGGSSSSSERSQPGAEGSPETPPGRCCRCCRAPRLLQAYSWKEEEEEDEGSMESLTSSEGEEPGSEVVIKMPMVDPEAQAPTKQPPRSSPNTVKRPTKKGRDRAGKGQKPRGKEQLAKRKTFSLVKEKKAARTLSAILLAFILTWTPYNIMVLVSTFCKDCVPETLWELGYWLCYVNSTINPMCYALCNKAFRDTFRLLLLCRWDKRRWRKIPKRPGSVHRTPSRQC

SEQ ID NO: 7 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 8 - protein, 382 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSEKSLALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 9 - protein, 422 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 10 - protein, 297 aa, Homo sapiens
MKHIINSYENINNTARNNSDCPRVVLPEEIFFTISIVGVLENLIVLLAVFKNKNLQAPMYFFICSLAISDMLGSLYKILENILIILRNMGYLKPRGSFETTADDIIDSLFVLSLLGSIFSLSVIAADRYITIFHALRYHSIVTMRRTVVVLTVIWTFCTGTGITMVIFSHHVPTVITFTSLFPLMLVFILCLYVHMFLLARSHTRKISTLPRANMKGAITLTILLGVFIFCWAPFVLHVLLMTFCPSNPYCACYMSLFQVNGMLIMCNAVIDPFIYAFRSPELRDAFKKMIFCSRYW

SEQ ID NO: 11 - protein, 418 aa, Homo sapiens
MRLNSSAPGTPGTPAADPFQRAQAGLEEALLAPGFGNASGNASERVLAAPSSELDVNTDIYSKVLVTAVYLALFVVGTVGNTVTAFTLARKKSLQSLQSTVHYHLGSLALSDLLTLLLAMPVELYNFIWVHHPWAFGDAGCRGYYFLRDACTYATALNVASLSVERYLAICHPFKAKTLMSRSRTKKFISAIWLASALLAVPMLFTMGEQNRSADGQHAGGLVCTPTIHTATVKVVIQVNTFMSFIFPMVVISVLNTIIANKLTVMVRQAAEQGQVCTVGGEHSTFSMAIEPGRVQALRHGVRVLRAVVIAFVVCWLPYHVRRLMFCYISDEQWTPFLYDFYHYFYMVTNALFYVSSTINPILYNLVSANFRHIFLATLACLCPVWRRRRKRPAFSRKADSVSSNHTLSSNATRETLY

SEQ ID NO: 12 - protein, 389 aa, Homo sapiens
MEGALAANWSAEAANASAAPPGAEGNRTAGPPRRNEALARVEVAVLCLILLLALSGNACVLLALRTTRQKHSRLFFFMKHLSIADLVVAVFQVLPQLLWDITFRFYGPDLLCRLVKYLQVVGMFASTYLLLLMSLDRCLAICQPLRSLRRRTDRLAVLATWLGCLVASAPQVHIFSLREVADGVFDCWAVFIQPWGPKAYITWITLAVYIVPVIVLAACYGLISFKIWQNLRLKTAAAAAAEAPEGAAAGDGGRVALARVSSVKLISKAKIRTVKMTFIIVLAFIVCWTPFFFVQMWSVWDANAPKEASAFIIVMLLASLNSCCNPWIYMLFTGHLFHELVQRFLCCSASYLKGRRLGETSASKKSNSSSFVLSHRSSSQRSCSQPSTA

SEQ ID NO: 13 - protein, 39 aa, Homo sapiens
DYKDDDDAMGQPGNGSAFLLAPNRSHAPDHDVTQQRDEV

SEQ ID NO: 14 - protein, 12 aa, Homo sapiens
RRSSLKHHHHHH

SEQ ID NO: 15 - protein, 8 aa, Homo sapiens
DYKDDDDA

SEQ ID NO: 16 - protein, 6 aa, Homo sapiens
HHHHHH

SEQ ID NO: 17 - protein, 490 aa, Homo sapiens
DYKDDDDAMGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIWTLCVIAVDRYFAITSPFKYQSLLTKNKARVIILMVWIVSGLTSFLPIQMHWYRATHQEAINCYAEETCCDFFTNQAYAIASSIVSFYVPLVIMVFVYSRVFQEAKRQLNIFEMLRIDEGLRLKIYKDTEGYYTIGIGHLLTKSPSLNAAKSELDKAIGRNTNGVITKDEAEKLFNQDVDAAVRGILRNAKLKPVYDSLDAVRRAALINMVFQMGETGVAGFTNSLRMLQQKRWDEAAVNLAKSRWYNQTPNRAKRVITTFRTGTWDAYKFCLKEHKALKTLGIIMGTFTLCWLPFFIVNIVHVIQDNLIRKEVYILLNWIGYVNSGFNPLIYCRSPDFRIAFQELLCLRRSSLKHHHHHH

SEQ ID NO: 18 - protein, 199 aa, Homo sapiens
WVVGMGIVMSLIVLAIVFGNVLVITAIAKFERLQTVTNYFITSLACADLVMGLAVVPFGAAHILMKMWTFGNFWCEFWTSIDVLCVTASIWTLCVIAVDRYFAITSPFKYQSLLTKNKARVIILMVWIVSGLTSFLPIQMHWYRATHQEAINCYAEETCCDFFTNQAYAIASSIVSFYVPLVIMVFVYSRVFQEAKRQL

SEQ ID NO: 19 - protein, 80 aa, Homo sapiens
KFCLKEHKALKTLGIIMGTFTLCWLPFFIVNIVHVIQDNLIRKEVYILLNWIGYVNSGFNPLIYCRSPDFRIAFQELLCL

SEQ ID NO: 20 - protein, 160 aa, Homo sapiens
NIFEMLRIDEGLRLKIYKDTEGYYTIGIGHLLTKSPSLNAAKSELDKAIGRNTNGVITKDEAEKLFNQDVDAAVRGILRNAKLKPVYDSLDAVRRAALINMVFQMGETGVAGFTNSLRMLQQKRWDEAAVNLAKSRWYNQTPNRAKRVITTFRTGTWDAY

SEQ ID NO: 21 - protein, 290 aa, Homo sapiens
IVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFR

SEQ ID NO: 22 - protein, 202 aa, Homo sapiens
SSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQ

SEQ ID NO: 23 - protein, 83 aa, Homo sapiens
STLQKEVHAAKSLAIIVGLFALCWLPLHIINCFTFFCPDCSHAPLWLMYLAIVLSHTNSVVNPFIYAYRIREFRQTFRKIIRS

SEQ ID NO: 24 - protein, 190 aa, Homo sapiens
KAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRE

SEQ ID NO: 25 - protein, 85 aa, Homo sapiens
LLKFSREKKAAKTLGIVVGCFVLCWLPFFLVMPIGSFFPDFKPSETVFKIVFWLGYLNSCINPIIYPCSSQEFKKAFQNVLRIQC

SEQ ID NO: 26 - protein, 194 aa, Homo sapiens
VAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENR

SEQ ID NO: 27 - protein, 82 aa, Homo sapiens
SLVKEKKAARTLSAILLAFILTWTPYNIMVLVSTFCKDCVPETLWELGYWLCYVNSTINPMCYALCNKAFRDTFRLLLLCRQ

SEQ ID NO: 28 - protein, 185 aa, Homo sapiens
YNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRR

SEQ ID NO: 29 - protein, 80 aa, Homo sapiens
LSQQKEKKATQMLAIVLGVFIICWLPFFITHILNIHCDCNIPPVLYSAFTWLGYVNSAVNPIIYTTFNIEFRKAFLKILH

SEQ ID NO: 30 - protein, 283 aa, Homo sapiens
KLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSENVALLKTVIIVLSVFIACWAPLFILLLLDVGCKVKTCDILFRAEYFLVLAVLNSGTNPIIYTLTNKEMRRAFIRIMSCS

SEQ ID NO: 31 - protein, 191 aa, Homo sapiens
YQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFR

SEQ ID NO: 32 - protein, 88 aa, Homo sapiens
MALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 33 - protein, 275 aa, Homo sapiens
PRVVLPEEIFFTISIVGVLENLIVLLAVFKNKNLQAPMYFFICSLAISDMLGSLYKILENILIILRNMGYLKPRGSFETTADDIIDSLFVLSLLGSIFSLSVIAADRYITIFHALRYHSIVTMRRTVVVLTVIWTFCTGTGITMVIFSHHVPTVITFTSLFPLMLVFILCLYVHMFLLARSHTRKISTLPRANMKGAITLTILLGVFIFCWAPFVLHVLLMTFCPSNPYCACYMSLFQVNGMLIMCNAVIDPFIYAFRSPELRDAFKKMIFCSRY

SEQ ID NO: 34 - protein, 205 aa, Homo sapiens
SKVLVTAVYLALFVVGTVGNTVTAFTLARKKSLQSLQSTVHYHLGSLALSDLLTLLLAMPVELYNFIWVHHPWAFGDAGCRGYYFLRDACTYATALNVASLSVERYLAICHPFKAKTLMSRSRTKKFISAIWLASALLAVPMLFTMGEQNRSADGQHAGGLVCTPTIHTATVKVVIQVNTFMSFIFPMVVISVLNTIIANKLTVM

SEQ ID NO: 35 - protein, 91 aa, Homo sapiens
PGRVQALRHGVRVLRAVVIAFVVCWLPYHVRRLMFCYISDEQWTPFLYDFYHYFYMVTNALFYVSSTINPILYNLVSANFRHIFLATLACL

SEQ ID NO: 36 - protein, 194 aa, Homo sapiens
LARVEVAVLCLILLLALSGNACVLLALRTTRQKHSRLFFFMKHLSIADLVVAVFQVLPQLLWDITFRFYGPDLLCRLVKYLQVVGMFASTYLLLLMSLDRCLAICQPLRSLRRRTDRLAVLATWLGCLVASAPQVHIFSLREVADGVFDCWAVFIQPWGPKAYITWITLAVYIVPVIVLAACYGLISFKIWQNL

SEQ ID NO: 37 - protein, 83 aa, Homo sapiens
VKLISKAKIRTVKMTFIIVLAFIVCWTPFFFVQMWSVWDANAPKEASAFIIVMLLASLNSCCNPWIYMLFTGHLFHELVQRFL

(19) United States
(12) Patent Application Publication
(10) Pub. No.: US 2011/0130543 A1
(43) Pub. Date: Jun. 2, 2011

(54) CHOLESTEROL CONSENSUS MOTIF OF MEMBRANE PROTEINS

(76) Inventors: Raymond C. Stevens, La Jolla, CA (US); Michael A. Hanson, San Marcos, CA (US); Vadim Cherezov, San Diego, CA (US)

(21) Appl. No.: 12/739,133

(22) PCT Filed: Oct. 22, 2008
(86) PCT No.: PCT/US08/80844

§ 371 Date: Sep. 23, 2010

Related U.S. Application Data
(60) Provisional application No. 60/999,951, filed on Oct. 22, 2007.
(60) Provisional application No. 61/000,325, filed on Oct. 24, 2007.
(60) Provisional application No. 61/060,107, filed on Jun. 9, 2008.

(51) Int. Cl. C07K 14/705; G06G 7/58

(52) U.S. Cl. 530/350; 703/12

(57) ABSTRACT

The invention provides the structure of a human β2-adrenergic receptor, a cholesterol consensus motif, and methods of identifying modulators of G-protein coupled receptors (GPCRs). Methods of using the modulators of the receptor, GPCRs, and the cholesterol consensus motif are also provided.

### CROSS REFERENCE TO RELATED APPLICATIONS

The present invention is related to and claims priority from U.S. Pat. Application Nos. 60/999,951, filed Oct. 22, 2007; 61/000,325, filed on Oct. 24, 2007; and 61/060,107, filed on Jun. 9, 2008, each of which is herein incorporated by reference, in its entirety, for all purposes.

### STATEMENT REGARDING FEDERALLY SPONSORED RESEARCH OR DEVELOPMENT

The U.S. Government has certain rights in this invention pursuant to Grant Nos. P50-GM073197 awarded by the National Institutes of Health.

### BACKGROUND OF THE INVENTION

1. Field of the Invention

The invention relates to the fields of chemistry, and biophysics.

2. Description of the Related Art

G-protein coupled receptors (GPCRs) comprise a broad class of membrane-bound proteins that share a variety of structural and functional attributes. See Friedricksson et al. Mol Pharmacol (63)6: p. 1256-1272, 2003; and Friedricksson et al. Mol Pharmacol (67)5: p. 1414-1425, 2005. GPCRs are classified into 1 of 6 classes: A, B, C, D, E, and F, see Friedricksson et al. (2003) and Friedricksson et al. (2005). GPCRs comprise seven transmembrane helical regions, as well as an extracellular portion that binds endogenous ligands. This extracelluar ligand binding domain is frequently a target site for pharmaceutical agents that modulate GPCR function. β₂-adrenergic receptor (β₂AR) (see SEQ ID NO: 1 for protein and SEQ ID NO: 2 for nucleotide sequences; see also accession number NM_{—}000024 on the NCBI web-site) is a well-characterized member of the class A GPCRs and is expressed in pulmonary and cardiac myocyte tissue (Milligan et al., 1994; Takeda et al., 2002). The β₂AR, and its close relative β₁-adrenergic receptor (β₁AR) can sense epinephrine and norepinephrine in bronchial vasculature and cardiac muscle, respectively, and a positive inotropic response can be elicited with β₁AR and bronchial dilation associated with β₂AR. One class of small molecules, known as beta-blockers, have been used clinically in the management of cardiac arrhythmias where they are thought to evoke their antagonistic effect mainly through β₁AR, but can also bind effectively to β₂AR. The high-resolution crystal structure of the β₂AR fused to T4-lysozyme (β₂AR-T4L) and a low-resolution partial structure of β₂AR in complex with a specific Fab fragment have recently been determined in the presence of the beta-blocker and partial inverse agonist carazolol. See Cherezov et al., 2007; Rasmussen et al., 2007; Rosenbaum et al., 2007; and co-owned U.S. Pat. Application Nos. 60/999,951, filed Oct. 22, 2007 and 61/000,325, filed on Oct. 24, 2007, all of which are herein incorporated by reference for all purposes. Timolol, a member of the first generation of beta-blockers, has found extensive use in the treatment of glaucoma by reducing intraocular pressure, as well as in the treatment of high-blood pressure and heart disease. Timolol has been characterized as a partial inverse agonist of the β₂AR where its efficacy varies from 28 to 90% inhibition of basal activity depending on the assay conditions. Timolol also has high water-solubility, thus making it a useful ligand for structural studies (Chidiac et al., 1994; Zimmerman and Kaufman, 1977).

Cholesterol is an important component regulating the structure and function of eukaryotic membranes and is thought to act primarily through the modulation of membrane fluidity and maintenance of sphingolipid rafts and membrane microdomains (Simons and Ikonen, 2000). In addition, cholesterol can play a regulatory role in a number of membrane proteins, either indirectly through its ability to modulate the physical properties of lipid membranes, or directly through specific interactions with select protein systems (Burger et al., 2000; Lee, 2004). It has been postulated that cholesterol can also play an important role in GPCR function and pharmacology where certain receptors are thought to partition into or out of cholesterol-rich caveolae (Pucadyil and Chattopadhyay, 2006). In addition, direct protein-cholesterol interactions have been demonstrated for the oxytocin, galanin, and serotonin_{1A} receptors (Gimpl et al., 1997; Pang et al., 1999; Pucadyil and Chattopadhyay, 2004). Despite these findings, the specific manner by which cholesterol binds to GPCRs and the effects exerted by such binding remain largely uncharacterized. There is thus a need for improved methods to characterize the three-dimensional structure of a cholesterol consensus motif (CCM) present in GPCRs, and to better structurally and biochemically characterize the interaction of molecules with the CCM, as well as to provide screening methods for developing GPCR modulators that bind to the CCM, and “bi-functional” ligands that bind the CCM and extracellular GPCR ligand-binding site(s). The present invention provides for these and other advantages as described below.

### SUMMARY OF THE INVENTION

Described herein is a method of identifying a compound that binds to a cholesterol consensus motif (CCM) of a G protein coupled receptor (GPCR) membrane protein by comparing a set of three-dimensional structures representing a set of candidate compounds with a three-dimensional molecular model of said CCM, including: receiving said three-dimensional model of said CCM, wherein said three-dimensional model of said CCM includes atomic co-ordinates of three or more residues selected from the set consisting of Ballesteros-Weinstein indexed residues [4.39-4.43(R,K)]---[4.50(W,Y)]---[4.46(I,V,L)]---[2.41(F,Y)]; receiving a set of compound three-dimensional models representing the set of candidate compounds, wherein each three-dimensional model includes atomic co-ordinates of a candidate compound of the set of candidate compounds; determining, for each of the set of compound three-dimensional models, a plurality of distance values indicating distances between the atomic co-ordinates of the candidate compound of the set of candidate compounds and the atomic coordinates of the three or more residues; determining, for each of the set of compound three-dimensional models, a binding strength value based on the plurality of distance values determined for the compound three-dimensional model, wherein the binding strength value indicates the stability of a complex formed by the GPCR membrane protein and a compound represented by the compound three-dimensional model; and storing a set of results indicating whether each candidate compound binds to the three-dimensional model based on the binding strength values.

In one embodiment of the method the GPCR membrane protein is selected from the group consisting of a class A GPCR, a class B GPCR, a class C GPCR, a class D GPCR, a class E GPCR, and a class F GPCR. In another embodiment of the method the set includes one or more members. In another embodiment the method further includes generating the three-dimensional molecular model of the cholesterol consensus motif (CCM).

In another embodiment the method, the method further includes generating the three-dimensional molecular model of the cholesterol consensus motif (CCM) includes: identifying an amino acid sequence of the G protein coupled receptor (GPCR) membrane protein; identifying the three or more residues of the amino acid sequence from the set consisting of Ballesteros-Weinstein indexed residues [4.39-4.43(R,K)]---[4.50(W,Y)]---[4.46(I,V,L)]---[2.41(F,Y)]; generating a three-dimensional model of the G protein coupled receptor (GPCR) membrane protein, the three-dimensional model of the G protein coupled receptor (GPCR) including atomic co-ordinates of residues in the amino acid sequence; and generating the three-dimensional molecular model of the cholesterol consensus motif (CCM) responsive to selecting the atomic co-ordinates of the three or more residues based on the generated three-dimensional model of the G protein coupled receptor (GPCR) membrane protein. In another embodiment, the method further includes generating the three-dimensional model of the G protein coupled receptor (GPCR) membrane protein using x-ray crystallography, electron crystallography, nuclear magnetic resonance, ab initio modeling, or a combination thereof. In another embodiment, the method further includes generating the three-dimensional model of the G protein coupled receptor (GPCR) membrane protein using computational protein structure modeling.

In another embodiment, the method further includes: receiving a three-dimensional model of a ligand binding site on the GPCR membrane protein, wherein the three-dimensional model of the ligand binding site includes atomic co-ordinates for a plurality of ligand-binding residues selected from a second set of Ballesteros-Weinstein indexed residues; determining, for each of the set of compound three-dimensional models, a plurality of distance values indicating distances between the atomic co-ordinates of the candidate compound of the set of candidate compounds and the atomic coordinates of the ligand-binding residues including the ligand binding site; determining, for each of the set of compound three-dimensional models, a second binding strength value based on the plurality of distance values determined for the compound three-dimensional model, wherein the second binding strength value indicates the stability of a complex formed by the GPCR membrane protein and a compound represented by the compound three-dimensional model; and storing a set of results indicating whether each candidate compound binds to the three-dimensional model based on the binding strength and second binding strength values.

In another embodiment, the GPCR membrane protein is β₂AR, and the second set of Ballesteros-Weinstein indexed residues are [3.32(D)]---[5.42(S)]---[5.43(S)]---[5.46(5)]---[6.44(F)]---[6.51(F)]---[6.52(F)]---[7.43(Y)]. In another embodiment, the GPCR membrane protein is HTR1A, and the second set of Ballesteros-Weinstein indexed residues are [3.32(D)]---[5.42(S)]---[5.43 (T)]---[5.46(A)]---[6.44(F)]---[6.51(F)]---[6.52(F)]---[7.43(Y)]. In another embodiment, the GPCR membrane protein is ADRA1A, and the second set of Ballesteros-Weinstein indexed residues are [3.32(D)]---[5.42(S)]---[5.43(A)]---[5.46(S)]---[6.44(F)]---[6.51(F)]---[6.52(F)]---[7.43(Y)]. In another embodiment, the GPCR membrane protein is ADORA2A, and the second set of Ballesteros-Weinstein indexed residues are [1.39(E)]---[3.36(T)]---[6.55(TN)]---[7.42(S)]---[7.43(H)]. In another embodiment, the GPCR membrane protein is CHRM1, and the second set of Ballesteros-Weinstein indexed residues are [3.32(D)]---[5.42(T)]---[5.43(A)]---[5.46(A)]---[6.44(F)]---[6.51(Y)]---[6.52(N)]---[7.43(Y)]. In another embodiment, the GPCR membrane protein is MC2R, and the second set of Ballesteros-Weinstein indexed residues are [3.25(D)]---[3.28(I)]---[3.29(D)]---[3.40(I)]---[4.56(T)]---[6.51(F)]---[6.52(F)]---[7.35(F)]. In another embodiment, the GPCR membrane protein is DRD2, and the second set of Ballesteros-Weinstein indexed residues are [3.32(D)]---[5.42(S)]---[5.43(S)]---[5.46(S)]---[6.44(F)]---[6.51(F)]---[6.52(F)]---[7.43(Y)]. In another embodiment, the GPCR membrane protein is EDG1, and the second set of Ballesteros-Weinstein indexed residues are [1.46(I)]---[2.57(Y)]---[2.65(G)]---[3.28(R)]---[3.29(E)]---[3.36(L)]---[6.44(F)]. In another embodiment, the GPCR membrane protein is TACR1, and the second set of Ballesteros-Weinstein indexed residues are [2.57(N)]---[2.61(N)]---[7.35(Y)]. In another embodiment, the GPCR membrane protein is NTSR1, and the second set of Ballesteros-Weinstein indexed residues are [1.39(Y)]---[1.42(L)]---[1.46(G)]---[6.51(Y)]---[6.54(R)]---[6.55(R)]---[7.35(Y)]---[7.43(Y)]. In another embodiment, the GPCR membrane protein is OXTR, and the second set of Ballesteros-Weinstein indexed residues are [2.61(Q)]---[3.28(V)]---[3.36(M)]---[5.38(Y)]---[6.44(F)]---[6.51(F)]---[6.52(F)]. In another embodiment, the GPCR membrane protein is any GPCR except ADORA2A. In another embodiment, the GPCR membrane protein is selected from the group consisting of any of the GPCRs specified above except ADORA2A.

In another embodiment of the method, the binding strength value is based on one or more of a hydrogen bonding strength, a hydrophobic interaction strength, or a Coulombic interaction binding strength. In another embodiment of the method, the second binding strength value is based on one or more of a hydrogen bonding strength, a hydrophobic interaction strength, or a Coulombic interaction binding strength.

In another embodiment of the method, one or more of the receiving, determining, or storing steps is carried out using a commercially-available software program. In another embodiment of the method, the commercially-available software program is selected from the group consisting of DOCK, QUANTA, Sybyl, CHARMM, AMBER, GRID, MCSS, AUTODOCK, CERIUS II, Flexx, CAVEAT, MACCS-3D, HOOK, LUDI, LEGEND, LeapFrog, Gaussian 92, QUANTA/CHARMM, Insight II/Discover, and ICM.

In another embodiment of the method, the set of candidate compounds includes one or more candidate compounds selected from the group consisting of:

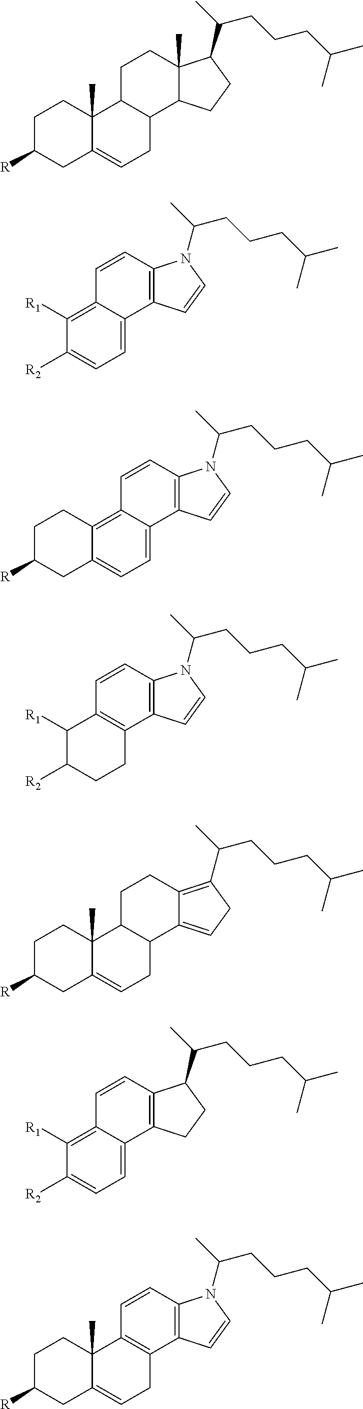

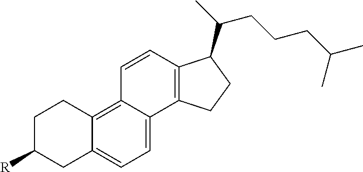

wherein R, R1, and R2 are independently selected from the group consisting of: hydrogen, acetate, aldehyde, benzoate, caproate, carboxylate, chloro, cyano, dichloroacetate, ethoxycarbonyl, ethyl ester, ethyleneketal, formate, hemisuccinate, hydrazone, oxime, phenylpropionate, proprionate, and sulphate.

In another embodiment the method further includes the step of contacting the GPCR membrane protein with a molecule including an identified candidate compound. In another embodiment of the method, the molecule further includes a moiety capable of competitively displacing a ligand from the GPCR membrane protein, wherein the ligand does not bind to the CCM. In another embodiment of the method, the ligand is selected from the group consisting of: timolol, isoproterenol, alprenolol, carazolol, and a ligand shown in Table 6.

In another embodiment of the method, the method further includes characterizing a binding interaction between the GPCR membrane protein and the molecule including the identified candidate compound, and storing a result of the characterizing. In another embodiment of the method, the characterization includes determining an activation of a function of the GPCR membrane protein, an inhibition of a function of the GPCR membrane protein, an increase in expression of the GPCR membrane protein, a decrease in expression of the GPCR membrane protein, a displacement of a sterol bound to the CCM, or a stability measure for the GPCR membrane protein.

In another embodiment of the method, the GPCR membrane protein is a class A GPCR membrane protein. In another embodiment of the method, the class A GPCR membrane protein is β₂AR. In another embodiment of the method, the class A GPCR membrane protein is selected from the group consisting of: TACR1, ADORA2A, ADRA1A, CHRM1, DRD2, EDG1, HTR1A, MC²R, NTSR1, and OXTR. In another embodiment of the method, the set of Ballesteros-Weinstein indexed residues for the CCM is [4.43(R)]---[4.50(W)]---[4.46(I)]---[2.41(Y)]. In another embodiment of the method, the class A GPCR membrane protein is HTR1A and the set of Ballesteros-Weinstein indexed residues for the CCM is [4.41(R)]---[4.50(W)]---[4.46(I)]---[2.41(Y)]. In another embodiment of the method, the class A GPCR membrane protein is ADRA1A and the set of Ballesteros-Weinstein indexed residues for the CCM is [4.41(R)]---[4.50(W)]---[4.46(L)]---[2.41(Y)]. In another embodiment of the method, the class A GPCR membrane protein is ADORA2A and the set of Ballesteros-Weinstein indexed residues for the CCM is [4.43(K)]---[4.50(W)]---[4.46(I)]---[2.41(Y)]. In another embodiment of the method, the class A GPCR membrane protein is CHRM1 and the set of Ballesteros-Weinstein indexed residues for the CCM is [4.41(R)]---[4.50(W)]---[4.46(I)]---[2.41(Y)]. In another embodiment of the method, the class A GPCR membrane protein is MC2R and the set of Ballesteros-Weinstein indexed residues for the CCM is [4.41(R)]---[4.50(W)]---[4.46(L)]---[2.41(F)]. In another embodiment of the method, the class A GPCR membrane protein is DRD2 and the set of Ballesteros-Weinstein indexed residues for the CCM is [4.41(R)]---[4.50(W)]---[4.46(I)]---[2.41(Y)]. In another embodiment of the method, the class A GPCR membrane protein is EDG1 and the set of Ballesteros-Weinstein indexed residues for the CCM is [4.41(R)]---[4.50(W)]---[4.46(I)]---[2.41(Y)]. In another embodiment of the method, the class A GPCR membrane protein is TACR1 and the set of Ballesteros-Weinstein indexed residues for the CCM is [4.43(K)]---[4.50(W)]---[4.46(I)]---[2.41(Y)]. In another embodiment of the method, the class A GPCR membrane protein is NTSR1 and the set of Ballesteros-Weinstein indexed residues for the CCM is [4.43(K)]---[4.50(W)]---[4.46(I)]---[2.41(Y)]. In another embodiment of the method, the class A GPCR membrane protein is OXTR and the set of Ballesteros-Weinstein indexed residues for the CCM is [4.43(R)]---[4.50(W)]---[4.46(V)]---[2.41(F)]. In another embodiment of the method, the GPCR membrane protein is any GPCR except ADORA2A. In another embodiment of the method, the GPCR membrane protein is selected from the group consisting of any of the GPCRs specified above except ADORA2A.

In another embodiment of the method, the class A GPCR membrane protein is selected from the group consisting of: HTR1A, HTR1B, HTR1E, HTR1F, HTR2A, HTR2B, HTR2c, HTR4, HTR6, HTR7, ADRA1A, ADRA1B, ADRA1D, ADORA2A, ADORA3, CHRM1, CHRM2, CHRM3, CHRM4, CHRM5, MC2R, ADRB2, DRD2, DRD3, DRD1, EDG1, EDG2, EDG3, GPR10, GPR19, GPR21, GPR52, MC3R, MC4R, MC5R, TACR1, TACR2, TACR3, NTSR1, NPY2R, OPN1SW, OPN1MW, OPN1LW, HCRTR1, HCRTR2, and OXTR. In another embodiment of the method, the class A GPCR membrane protein is selected from the group consisting of: HTR5A, ADRA2A, ADRA2B, ADRA2C, ADORA1, ADRB1, ADRB3, CNR2, CCKAR, DRD4, DRD5, EDNRB, FPR1, GALR1, GALR2, GALR3, CCKBR, GHSR, GPR45, GPR63, GPR72, GPR1, GPR3, GNRHR, HRH1, HRH2, LGR7, MTNR1A, MTNR1B, GPR50, MC1R, MTLR1, NPFF1, NPGPR, TACR3L, NTSR2, NPY1R, OR10H1, OR10H2, OR10H3, OR10J1, OR11A1, OPN4, LTB4R, PTGER3, PTGER4, PTGFR, TBXA2R, TRHR, and AVPR1A.)]. In another embodiment of the method, the class A GPCR membrane protein is any GPCR except ADORA2A. In another embodiment of the method, the class A GPCR membrane protein is selected from the group consisting of any of the GPCRs specified above except ADORA2A.

In another embodiment of the method, the set of candidate compounds is designed from known compounds. In another embodiment of the method, the set of candidate compounds is designed de novo based on the three-dimensional molecular model of the CCM.

Also described herein is a crystalline form of β₂AR(E122W)-T4L having unit cell dimensions of a=40.0 Angstroms, b=75.7 Angstroms, and c=172.7 Angstroms.

In one embodiment, the space group of the crystalline form is P2₁2₁2₁. In another embodiment the crystalline form diffracts X-rays to resolution of 2.8 Angstroms.

Also described herein is a ligand which cross reacts with a compound that binds a CCM of a GPCR membrane protein, wherein the CCM includes three or more residues selected from the set consisting of Ballesteros-Weinstein indexed residues [4.39-4.43(R,K)]---[4.50(W,Y)]---[4.46(I,V,L)]---[2.41(F,Y)], which compound adopts one or more copies of a motif that includes: one or more of a ring CH-π electron; an aromatic group; a plurality of hydrophobic groups; or a hydrogen bond donor, with the proviso that the ligand is not cholesterol, cholesteryl hemisuccinate, or salmeterol.

In one embodiment of the ligand, the ligand further includes a moiety capable of selectively displacing a ligand from the GPCR membrane protein, wherein the ligand does not bind to the CCM. In another embodiment of the ligand, the ligand is selected from the group consisting of: timolol, isoproterenol, alprenolol, carazolol, and a ligand shown in Table 6. In another embodiment of the ligand, the motif further includes a second ring CH-π electron. In another embodiment of the ligand, the plurality is three hydrophobic groups.

In another embodiment of the ligand, the GPCR membrane protein is a class A GPCR membrane protein. In another embodiment of the ligand, the class A GPCR membrane protein is β₂AR. In another embodiment of the ligand, the class A GPCR membrane protein is selected from the group consisting of: HTR1A, HTR1B, HTR1E, HTR1F, HTR2A, HTR2B, HTR2c, HTR4, HTR6, HTR7, ADRA1A, ADRA1B, ADRA1D, ADORA2A, ADORA3, CHRM1, CHRM2, CHRM3, CHRM4, CHRM5, MC2R, ADRB2, DRD2, DRD3, DRD1, EDG1, EDG2, EDG3, GPR10, GPR19, GPR21, GPR52, MC3R, MC4R, MC5R, TACR1, TACR2, TACR3, NTSR1, NPY2R, OPN1SW, OPN1MW, OPN1LW, HCRTR1, HCRTR2, OXTR, HTR5A, ADRA2A, ADRA2B, ADRA2C, ADORA1, ADRB1, ADRB3, CNR2, CCKAR, DRD4, DRD5, EDNRB, FPR1, GALR1, GALR2, GALR3, CCKBR, GHSR, GPR45, GPR63, GPR72, GPR1, GPR3, GNRHR, HRH1, HRH2, LGR7, MTNR1A, MTNR1B, GPR50, MC1R, MTLR1, NPFF1, NPGPR, TACR3L, NTSR2, NPY1R, OR10H1, OR10H2, OR10H3, OR10J1, OR11A1, OPN4, LTB4R, PTGER3, PTGER4, PTGFR, TBXA2R, TRHR, and AVPR1A.)]. In another embodiment of the method, the class A GPCR membrane protein is any GPCR except ADORA2A. In another embodiment of the ligand, the class A GPCR membrane protein is selected from the group consisting of any of the GPCRs specified above except ADORA2A.

Also described herein is a method of identifying a compound that binds to a ligand binding site of a G protein coupled receptor (GPCR) membrane protein by comparing a set of three-dimensional structures representing a set of candidate compounds with a three-dimensional molecular model of the ligand binding site, including: receiving a three-dimensional model of a ligand binding site on the GPCR membrane protein, wherein the three-dimensional model of the ligand binding site includes atomic co-ordinates for a plurality of ligand-binding residues selected from a set of Ballesteros-Weinstein indexed residues; determining, for each of the set of compound three-dimensional models, a plurality of distance values indicating distances between the atomic co-ordinates of the candidate compound of the set of candidate compounds and the atomic coordinates of the ligand-binding residues including the ligand binding site; determining, for each of the set of compound three-dimensional models, a binding strength value based on the plurality of distance values determined for the compound three-dimensional model, wherein the binding strength value indicates the stability of a complex formed by the GPCR membrane protein and a compound represented by the compound three-dimensional model; and storing a set of results indicating whether each candidate compound binds to the three-dimensional model based on the binding strength values.

In one embodiment of the method, the GPCR membrane protein is selected from the group consisting of a class A GPCR, a class B GPCR, a class C GPCR, a class D GPCR, a class E GPCR, and a class F GPCR. In another embodiment of the method, the set includes one or more members.

In another embodiment of the method, the GPCR membrane protein is β₂AR, and the set of Ballesteros-Weinstein indexed residues are [3.32(D)]---[5.42(S)]---[5.43(S)]---[5.46(S)]---[6.44(F)]---[6.51(F)]---[6.52(F)]---[7.43(Y)]. In another embodiment of the method, the GPCR membrane protein is HTR1A, and the second set of Ballesteros-Weinstein indexed residues are [3.32(D)]---[5.42(S)]---[5.43(T)]---[5.46(A)]---[6.44(F)]---[6.51(F)]---[6.52(F)]---[7.43(Y)]. In another embodiment of the method, the GPCR membrane protein is ADRA1A, and the second set of Ballesteros-Weinstein indexed residues are [3.32(D)]---[5.42(S)]---[5.43(A)]---[5.46(S)]---[6.44(F)]---[6.51(F)]---[6.52(F)]---[7.43(Y)]. In another embodiment of the method, the GPCR membrane protein is ADORA2A, and the second set of Ballesteros-Weinstein indexed residues are [1.39(E)]---[3.36(T)]---[6.55(TN)]---[7.42(S)]---[7.43(H)]. In another embodiment of the method, the GPCR membrane protein is CHRM1, and the second set of Ballesteros-Weinstein indexed residues are [3.32(D)]---[5.42(T)]---[5.43(A)]---[5.46(A)]---[6.44(F)]---[6.51(Y)]---[6.52(N)]---[7.43(Y)]. In another embodiment of the method, the GPCR membrane protein is MC2R, and the second set of Ballesteros-Weinstein indexed residues are [3.25(D)]---[3.28(I)]---[3.29(D)]---[3.40(I)]---[4.56(T)]---[6.51(F)]---[6.52(F)]---[7.35(F)]. In another embodiment of the method, the GPCR membrane protein is DRD2, and the second set of Ballesteros-Weinstein indexed residues are [3.32(D)]---[5.42(S)]---[5.43(S)]---[5.46(S)]---[6.44(F)]---[6.51(F)]---[6.52(F)]---[7.43(Y)]. In another embodiment of the method, the GPCR membrane protein is EDG1, and the second set of Ballesteros-Weinstein indexed residues are [1.46(I)]---[2.57(Y)]---[2.65(G)]---[3.28(R)]---[3.29(E)]---[3.36(L)]---[6.44(F)]. In another embodiment of the method, the GPCR membrane protein is TACR1, and the second set of Ballesteros-Weinstein indexed residues are [2.57(N)]---[2.61(N)]---[7.35(Y)]. In another embodiment of the method, the GPCR membrane protein is NTSR1, and the second set of Ballesteros-Weinstein indexed residues are [1.39(Y)]---[1.42(L)]---[1.46(G)]---[6.51(Y)]---[6.54(R)]---[6.55(R)]---[7.35(Y)]---[7.43(Y)]. In another embodiment of the method, the GPCR membrane protein is OXTR, and the second set of Ballesteros-Weinstein indexed residues are [2.61(Q)]---[3.28(V)]---[3.36(M)]---[5.38(Y)]---[6.44(F)]---[6.51(F)]---[6.52(F)]. In another embodiment of the method, the GPCR membrane protein is any GPCR except ADORA2A. In another embodiment of the ligand, the GPCR membrane protein is selected from the group consisting of any of the GPCRs specified above except ADORA2A.

In another embodiment of the method, the binding strength value is based on one or more of a hydrogen bonding strength, a hydrophobic interaction strength, or a Coulombic interaction binding strength.

In another embodiment of the method, one or more of the receiving, determining, or storing steps is carried out using a commercially-available software program. In another embodiment of the method, the commercially-available software program is selected from the group consisting of DOCK, QUANTA, Sybyl, CHARMM, AMBER, GRID, MCSS, AUTODOCK, CERIUS II, Flexx, CAVEAT, MACCS-3D, HOOK, LUDI, LEGEND, LeapFrog, Gaussian 92, QUANTA/CHARMM, Insight II/Discover, and ICM.

In another embodiment of the method, the method further includes the step of contacting the GPCR membrane protein with a molecule including an identified candidate compound. In another embodiment of the method, the molecule further includes a moiety capable of competitively displacing a ligand from the GPCR membrane protein, wherein the ligand binds to a CCM.

In another embodiment of the method, the method further includes characterizing a binding interaction between the GPCR membrane protein and the molecule including the identified candidate compound, and storing a result of the characterizing. In another embodiment of the method, the characterization includes determining an activation of a function of the GPCR membrane protein, an inhibition of a function of the GPCR membrane protein, an increase in expression of the GPCR membrane protein, a decrease in expression of the GPCR membrane protein, a displacement of a ligand bound to the ligand binding site, or a stability measure for the GPCR membrane protein.

In another embodiment of the method, the GPCR membrane protein is a class A GPCR membrane protein. In another embodiment of the method, the class A GPCR membrane protein is β₂AR. In another embodiment of the method, the class A GPCR membrane protein is selected from the group consisting of: TACR1, ADORA2A, ADRA1A, CHRM1, DRD2, EDG1, HTR1A, MC2R, NTSR1, and OXTR. In another embodiment of the method, the class A GPCR membrane protein is selected from the group consisting of: HTR1A, HTR1B, HTR1E, HTR1F, HTR2A, HTR2B, HTR2c, HTR4, HTR6, HTR7, ADRA1A, ADRA1B, ADRA1D, ADORA2A, ADORA3, CHRM1, CHRM2, CHRM3, CHRM4, CHRM5, MC2R, ADRB2, DRD2, DRD3, DRD1, EDG1, EDG2, EDG3, GPR10, GPR19, GPR21, GPR52, MC3R, MC4R, MC5R, TACR1, TACR2, TACR3, NTSR1, NPY2R, OPN1SW, OPN1MW, OPN1LW, HCRTR1, HCRTR2, and OXTR. In another embodiment of the method, the class A GPCR membrane protein is selected from the group consisting of: HTR5A, ADRA2A, ADRA2B, ADRA2C, ADORA1, ADRB1, ADRB3, CNR2, CCKAR, DRD4, DRD5, EDNRB, FPR1, GALR1, GALR2, GALR3, CCKBR, GHSR, GPR45, GPR63, GPR72, GPR1, GPR3, GNRHR, HRH1, HRH2, LGR7, MTNR1A, MTNR1B, GPR50, MC1R, MTLR1, NPFF1, NPGPR, TACR3L, NTSR2, NPY1R, OR10H1, OR10H2, OR10H3, OR10J1, OR11A1, OPN4, LTB4R, PTGER3, PTGER4, PTGFR, TBXA2R, TRHR, and AVPR1A. In another embodiment of the method, the class A GPCR membrane protein is any GPCR except ADORA2A. In another embodiment of the ligand, the class A GPCR membrane protein is selected from the group consisting of any of the GPCRs specified above except ADORA2A

In another embodiment of the method, the set of candidate compounds is designed from known compounds. In another embodiment of the method, the set of candidate compounds is designed de novo based on the three-dimensional molecular model of the ligand binding site.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1. Overview of the ^{tim}β₂AR(E122W)-T4L structure. A. The location of the tryptophan mutation is indicated, as well as the position of the ligand timolol. Two cholesterol molecules (orange) occupy roughly the same position as in the ^{car}β₂AR-T4L structure. Two lipid monoolein molecules (yellow) are located in the proximity of the E122^{3.41}W mutation in a crystal packing interface and one by helix I. The tilt angle between the receptor and T4L is markedly different between the ^{tim}β₂AR(E122W)-T4L structure (green ribbon trace) and ^{car}β₂AR-T4L (blue ribbon trace). This intramolecular conformational change is likely due to an altered crystal packing environment. B. Timolol and carazolol are both shown and colored magenta and green, respectively. Intra-receptor polar interactions are represented by black dashed lines and receptor-ligand polar interactions by red dashed lines. In addition to the interactions between Asp113^{3.32}, Asn312^{7.39} and Tyr316^{7.43} and the oxypropanolamine tail of the both ligands, the morpholino oxygen of timolol is within hydrogen bonding distance of Asn293^{6.55} which has an altered side-chain rotamer relative to the carazolol-bound structure (orange side-chains). Thus, the head group of timolol participates in a second hydrogen bonding network between Tyr308^{7.35}, Asn293^{6.55} and Ser204^{5.43}. In addition, the thiadiazole ring protrudes deeper into the binding pocket than the analogous ring of carazolol allowing a stronger interaction between the thiadiazole group and Thr118^{3.37}.

FIG. 2. Structural evidence for cholesterol specificity in binding. A. Crystal packing environment of ^{car}β₂AR-T4L (2RH1) where the receptor monomers pack in a parallel orientation. Three cholesterol molecules are bound to each monomer and a palmitic acid alkyl chain from the crystallographically related monomer that is located between cholesterol two and three. The four lipid molecules form an eight membered lipid sheet when the crystallographically related monomer is generated. B. Crystal packing environment of ^{tim}β₂AR(E122W)-T4L structure where the receptor monomers pack in an antiparallel orientation. Cholesterol 1 and 2 are retained in the new crystal form and are not implicated in packing interactions. C. Experimental electron density for the cholesterol molecules is shown in stereo. The Fₒ-F_{c} maps contoured at 2σ were calculated after omitting the cholesterol contribution to the overall phases and randomly shaking the model to reduce phase bias. D. Comparison of cholesterol binding between ^{tim}β₂AR(E122W)-T4L (yellow) and ^{car}β₂AR-T4L (cyan). Cholesterol two binds in approximately the same orientation between the two structures. Cholesterol one is modeled differently in the current structure with a 90° rotation and a 1.9 Å translation about the long axis of the molecule. These modifications were necessary to optimally fit the experimental electron density.

FIG. 3. Analysis of helical packing and thermal stability increase due to cholesterol binding. A. Receptor is colored by normalized occluded surface area. Red thick lines indicate the compact areas of the receptor and blue thin lines are the least compact. Helix IV has the lowest packing of the seven helices in the tertiary structure, particularly on the cytoplasmic end. Cholesterol binding stabilizes the receptor by increasing packing constraints, especially in the vicinity of the cytoplasmic end of helix IV. The values range from ten to seventy percent of the total available surface area being involved in packing interactions. B. Differences in the normalized occluded surface area of the receptor due to cholesterol binding. Values range from zero to fifteen percent increase in packing of available surface area due to cholesterol binding with the most significant increases seen for residues on helices II and IV. C. Molecular surface representation of the receptor and cholesterol. Green colored surface corresponds to atoms on both cholesterol and receptor that are within 4 Å of each other. Blue colored surface corresponds to atoms on the receptor that are 4 and 5 Å from the cholesterol molecules. In the second panel, the cholesterol molecules have been lifted out of the binding groove to better show the interactions and the binding groove. D. Isothermal CPM determination of the half-life of denaturation in the presence of 1M GnHCl with and without both CHS and timolol. The thickness of the line represents the 95% confidence interval over three replicates and the fitted half lives are indicated next to the respective curves. Both timolol and cholesterol cause an approximate 5-fold increase in half-life under these conditions. In combination, the effect is almost 16-fold relative to apo.

FIG. 4. The structurally determined receptor cholesterol consensus motif and the effects of cholesterol association on ligand binding properties of β₂AR. A. The sites of importance in the receptor cholesterol consensus motif are displayed with the β₂AR side-chain positions. Site 1 (colored orange) on helix II at position 2.41 can be either a phenylalanine or tyrosine. Site 2 (colored blue) at the cytoplasmic base of helix IV spanning positions 4.39-4.43 fulfills the CCM requirement if one or more of these positions contains an arginine or lysine residue. Site 3 (colored green) at position 4.46 on helix IV contributes van der Waals interactions (represented as space-filling atoms) to cholesterol binding and fulfills the CCM requirement if isoleucine, valine or leucine occupy the position. Site 4 (colored cyan) at position 4.50 on helix IV contributes CH-n hydrogen bonding interactions (represented as space-filling atoms) and is the most conserved site with tryptophan occupying the position in 94% of class A receptors. B. Competition binding curves for β₂AR(E122W)-T4L in the presence and absence of cholesterol. Cholesterol in complex with β-methyl cyclodextran was added to the expression cultures 24 hours post expression. A two-fold reduction in the Kᵢ for timolol is observed due to cholesterol addition, but not for isoproterenol.

FIG. 5. Venn diagram illustrating the abundance of specific elements of the CCM among human class A GPCRs. The individual circles are proportional to the percentage of receptors possessing each element. Twenty-one percent of human class A receptors contain the entire four component CCM. If the requirement for an aromatic at position 2.41 is removed, 44% of human class A receptors would contain the revised CCM (rCCM) motif. The removal of this position from the CCM is justified by the relatively long van der Waals interactions between cholesterol and Tyr70 in β₂AR.

FIG. 6. Ligand-based pharmacophore model based on the interactions between cholesterol and β₂AR. CHπ refers to one or more CHπ electrons.

FIG. 7. Distance constraints between select regions of the sites and points of the pharmacophore model.

FIG. 8. Angle constraints between select regions of the sites of the pharmacophore model. Each angle is represented by the angle defined by Site 1A (far left sphere of Site 1) and the line projected along the direction of the ring edge interactions associated with Site 1.

FIG. 9. Mapping of cholesterol onto the pharmacophore model.

FIG. 10. Modeled binding of salmeterol to β₂AR. A. Molecular structure of salmeterol with its three distinct entities marked as the orthosteric binding moiety, alkyl chain linker, and exosite binding moiety. B. Salmeterol docked into the β₂AR structure where the orthosteric binding moiety is making optimal interactions with its biochemically determined anchor points: Ser207, Ser 203, Asp113, Asn312, and Tyr316. C. Salmeterol docked into the β₂AR structure so that the exosite binding phenyl ring is located in the vicinity of the biochemically determined interaction site at the cytoplasmic base of helix IV. D. Both ligand positions are shown where it is apparent based on the modeling that an alkyl chain two carbon units longer can make optimal interactions with both sites simultaneously.

FIG. 11. A general diagram of a CCM site binding moiety linked by a linker to a ligand binding site binding moiety.

FIG. 12. Example of CHS-induced stabilization as judged by size exclusion chromatography.

FIG. 13. The androstenol molecules depicted in the figure are expected to bind to the CCM motif of GPCRs.

FIG. 14. R groups designate the potential for derivatization of the sterol ring structure. “L(1-3)” in upper left figure indicates that the ring may be expanded at those positions by the addition of 1-3 ring members.

### DETAILED DESCRIPTION OF THE INVENTION

### Advantages and Utility

Briefly, and as described in more detail below, described herein is the 2.8 Angstrom structure of a human β2-adrenergic receptor, which includes a cholesterol consensus motif (CCM). Advantages of this invention can include: the ability to create or identify compounds with increased specificity for proteins and an increased cholesterol context-specific action of compounds. While much of the disclosure that follows deals specifically with a human β2AR, the invention contemplates and encompasses application of findings and observations developed using this receptor to other GPCRs having a CCM.

### DEFINITIONS

Terms used in the claims and specification are defined as set forth below unless otherwise specified.

As used herein, the term “binding site” or “binding pocket” refers to a region of a protein that binds or interacts with a particular compound.

As used herein, the terms “binding” or “interaction” refers to a condition of proximity between a chemical entity, compound, or portions thereof, with another chemical entity, compound or portion thereof. The association or interaction can be non-covalent—wherein the juxtaposition is energetically favored by hydrogen bonding or van der Waals or electrostatic interactions—or it can be covalent.

As used herein, the term “residue” refers to an amino acid residue is one amino acid that is joined to another by a peptide bond. Residue is referred to herein to describe both an amino acid and its position in a polypeptide sequence.

As used herein, the term “surface residue” refers to a surface residue is a residue located on a surface of a polypeptide. In contrast, a buried residue is a residue that is not located on the surface of a polypeptide. A surface residue usually includes a hydrophilic side chain. Operationally, a surface residue can be identified computationally from a structural model of a polypeptide as a residue that contacts a sphere of hydration rolled over the surface of the molecular structure. A surface residue also can be identified experimentally through the use of deuterium exchange studies, or accessibility to various labeling reagents such as, e.g., hydrophilic alkylating agents.

As used herein, the term “polypeptide” refers to a single linear chain of 2 or more amino acids. A protein is an example of a polypeptide.

As used herein, the term “homolog” refers to a gene related to a second gene by descent from a common ancestral DNA sequence. The term, homolog, can apply to the relationship between genes separated by the event of speciation or to the relationship between genes separated by the event of genetic duplication.

As used herein, the term “conservation” refers to conservation a high degree of similarity in the primary or secondary structure of molecules between homologs. This similarity is thought to confer functional importance to a conserved region of the molecule. In reference to an individual residue or amino acid, conservation is used to refer to a computed likelihood of substitution or deletion based on comparison with homologous molecules.

As used herein, the term “distance matrix” refers to the method used to present the results of the calculation of an optimal pairwise alignment score. The matrix field (i,j) is the score assigned to the optimal alignment between two residues (up to a total of i by j residues) from the input sequences. Each entry is calculated from the top-left neighboring entries by way of a recursive equation.

As used herein, the term “substitution matrix” refers to a matrix that defines scores for amino acid substitutions, reflecting the similarity of physicochemical properties, and observed substitution frequencies. These matrices are the foundation of statistical techniques for finding alignments.

As used herein, the term “pharmacophore” refers to an ensemble of steric and electronic features that is necessary to ensure the optimal supramolecular interactions with a specific biological target structure and to trigger or block a biological response. A pharmacophore can be used to design one or more candidate compounds that comprise all or most of the ensemble of steric and electronic features present in the pharmacophore and that are expected to bind to a site and trigger or block a biological response.

As used herein, the term “atomic co-ordinates” refers to a set of three-dimensional co-ordinates for atoms within a molecular structure. In one embodiment, atomic-co-ordinates are obtained using X-ray crystallography according to methods well-known to those of ordinarily skill in the art of biophysics. Briefly described, X-ray diffraction patterns can be obtained by diffracting X-rays off a crystal. The diffraction data are used to calculate an electron density map of the unit cell comprising the crystal; said maps are used to establish the positions of the atoms (i.e., the atomic co-ordinates) within the unit cell. Those of skill in the art understand that a set of structure co-ordinates determined by X-ray crystallography contains standard errors. In other embodiments, atomic co-ordinates can be obtained using other experimental biophysical structure determination methods that can include electron diffraction (also known as electron crystallography) and nuclear magnetic resonance (NMR) methods. In yet other embodiments, atomic co-ordinates can be obtained using molecular modeling tools which can be based on one or more of ab initio protein folding algorithms, energy minimization, and homology-based modeling. These techniques are well known to persons of ordinary skill in the biophysical and bioinformatic arts, and are described in greater detail below.

Atomic co-ordinates for binding pockets, such as, e.g., the GPCRs CCMs, and agonist/antagonist binding sites of the present invention are intended to encompass those co-ordinates set out in the .pdb files (Appendices I-XI) incorporated into this specification, as well as co-ordinates that are substantially equivalent. Substantially equivalent co-ordinates are those that can be related to a reference set of co-ordinates by transformation reflecting differences in the choice of origin or inter-axis angels for one or more axes used to define the coordinate system. Operationally, co-ordinates are “substantially equivalent” when the structures represented by those co-ordinates can be superimposed in a manner such that root mean square deviations (RMSD) of atomic positions for the structures differs by less than a predetermined threshold. In some embodiments that threshold is less than about 5 Angstroms, or less than about 4 Angstroms, or less than about 3 Angstroms, or less than about 2 Angstroms, or less than about 1 Angstrom, or less than about 0.9 Angstrom, or less than about 0.8 Angstrom, or less than about 0.7 Angstrom, or less than about 0.6 Angstrom, or less than about 0.5 Angstrom, or less than about 0.4 Angstrom, or less than about 0.3 Angstrom. Preferably, co-ordinates are considered “substantially equivalent” when the RMSD is less than about 1 Angstrom. Methods for structure superpositioning and RMSD calculations are well known to those of ordinary skill in the art, and can be carried out using programs such as, e.g., the programs shown in Table 9 below.

Structural similarity can be inferred from, e.g., sequence similarity, which can be determined by one of ordinary skill through visual inspection and comparison of the sequences, or through the use of well-known alignment software programs such as CLUSTAL (Wilbur, W. J. and Lipman, D. J. Proc. Natl. Acad. Sci. USA, 80, 726 730 (1983)) or CLUSTALW (Thompson, J. D., Higgins, D. G. and Gibson, T. J., CLUSTAL W: improving the sensitivity of progressive multiple sequence alignment through sequence weighting, positions-specific gap penalties and weight matrix choice, Nucleic Acids Research, 22:4673 4680 (1994)) or BLAST® (Altschul S F, Gish W, et al., J. Mol. Biol., October 5; 215(3):403 10 (1990)), a set of similarity search programs designed to explore all of the available sequence databases regardless of whether the query is protein or DNA. CLUSTAL W is available at the EMBL-EBI website (http://www.ebi.ac.uk/clustalw/); BLAST is available from the National Center for Biotechnology website (http://www.ncbi.nlm.nih.gov/BLAST/). A residue within a first protein or nucleic acid sequence corresponds to a residue within a second protein or nucleic acid sequence if the two residues occupy the same position when the first and second sequences are aligned.

The term “a set” refers to a collection of one or more objects.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence co-ordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (NCBI web-site)

The term “sterol” refers to a subgroup of steroids with a hydroxyl group at the 3-position of the A-ring. See Fahy E. Subramaniam S et al., “A comprehensive classification system for lipids,” J. Lipid Res. 46 (5):839-861 (2005)). Sterols are amphipathic lipids synthesized from acetyl-coenzyme A via the HMG-CoA reductase pathway. The overall molecule is quite flat. Sterols can include, e.g., cholesterol or CHS.

The term “atomic co-ordinates for residues” refers to co-ordinates for all atoms associated with a residue, or for some of the atoms such as, e.g., side chain atoms.

The term “atomic co-ordinates of a candidate compound” refers to co-ordinates for all atoms comprising the compound or a subset of atoms comprising the compound.

The term “characterizing a binding interaction” refers to characterizing any observable property of a first molecule and determining an whether there is a change in that observable property after contacting the first molecule with a second molecule under conditions in which said first and second molecules can potentially bind.

Ballesteros-Weinstein numbering is used throughout the text as superscripts to the protein numbering. Within each helix is a single most conserved residue among the class A GPCRs. This residue is designated X.50, where x is the number of the transmembrane helix. All other residues on that helix are numbered relative to this conserved position.

It must be noted that, as used in the specification and the appended claims, the singular forms “a,” “an” and “the” include plural referents unless the context clearly dictates otherwise.

Introduction

G-protein coupled receptors are cell surface receptors that indirectly transduce extracellular signals to downstream effectors, e.g., intracellular signaling proteins, enzymes, or channels. G-protein coupled receptor membrane proteins are grouped into one of 6 classes: A, B, C, D, E, and F, see Friedricksson et al. (2003) and Friedricksson et al. (2005), supra. An example of a mammalian G-protein coupled receptor is β₂-adrenergic receptor (β₂AR). Changes in the activity of these effectors then mediate subsequent cellular events. The interaction between the receptor and the downstream effector is mediated by a G-protein, a heterotrimeric protein that binds GTP. Examples of mammalian G proteins include Gi, Go, Gq, Gs, and Gt.

G-protein coupled receptors (GPCRs) typically have seven transmembrane regions, along with an extracellular domain and a cytoplasmic tail at the C-terminus. These receptors form a large superfamily of related receptor molecules that play a key role in many signaling processes, such as sensory and hormonal signal transduction. Human β₂AR is an example of a class A GPCR.

The role of cholesterol in eukaryotic membrane protein function has been primarily attributed to an influence on membrane fluidity and curvature. A 2.8 Å resolution crystal structure of a thermally-stabilized human β₂-adrenergic receptor bound to cholesterol and the partial inverse agonist timolol is described. The receptors pack as monomers in an antiparallel association with two distinct cholesterol molecules bound per receptor, but not in the packing interface, thereby indicating a structurally relevant cholesterol binding site between helices I, II, III, and IV. Thermal stability analysis using isothermal denaturation confirms that cholesterol enhances the stability of the receptor. A cholesterol consensus motif (CCM) is defined that predicts cholesterol binding for 26% of all class A receptors indicating that specific sterol binding is important to the structure and stability of other G protein-coupled receptors, and that this site can provide a novel target for allosteric therapeutic discovery. Class A GPCRs function in a variety of physiological processes such as vasodilation, bronchodilation, neurotransmitter signaling, stimulation of endocrine secretions, gut peristalsis, development, mitogenesis, cell proliferation, cell migration, immune system function, and oncogenesis. Accordingly, class A GPCRs can be used as screening targets to identify modulators of these processes which can then function to ameliorate diseases associated with these processes, e.g., cancer and autoimmunity.

The Crystal Structure Co-ordinates of the Human β2-Adrenergic Receptor and the CCM

The 2.8 Angstrom structure of human 132-adrenergic receptor and the CCM can be used as a model for rationally designing pharmacophore and/or candidate compounds, either de novo or by modification of known compounds. As noted below, the CCM is a motif that is conserved across a large number of G protein coupled receptors (GPCRs) indicating that the 2.8 Angstrom structure of human β2-adrenergic receptor can be used for rational design of CCM-specific compounds to CCM-comprising GPCRs other than human β2-adrenergic receptor, including GPCRs belonging to any of classes A-E. Pharmacophore and candidate compounds identified through the use of the crystal structure co-ordinates are useful for altering the binding of cholesterol or other agents to a CCM, and so have utility as pharmaceuticals. Pharmacophores and candidate compounds can be determined according to any method known in the art, including the methods described in U.S. Pat. No. 5,888,738 to Hendry, and the methods described in U.S. Pat. No. 5,856,116 to Wilson et al. the disclosures of which both are incorporated by reference in their entirety for all purposes.

The structure data provided herein can be used in conjunction with computer-modeling techniques to develop models of sites on the human β2-adrenergic receptor or CCM on this or other GPCRs selected by analysis of the crystal structure data. The site models characterize the three-dimensional topography of site surface, as well as factors including van der Waals contacts, electrostatic interactions, and hydrogen-bonding opportunities. Computer simulation techniques can be used to map interaction positions for functional groups including protons, hydroxyl groups, amine groups, divalent cations, aromatic and aliphatic functional groups, amide groups, alcohol groups, etc. that are designed to interact with the model site. These groups can be designed into a pharmacophore or candidate compound with the expectation that the candidate compound will specifically bind to the site. Pharmacophore design thus involves a consideration of the ability of the candidate compounds falling within the pharmacophore to interact with a site through any or all of the available types of chemical interactions, including hydrogen bonding, van der Waals, electrostatic, and covalent interactions, although, in general, and preferably, pharmacophores interact with a site through non-covalent mechanisms.

The ability of a pharmacophore or candidate compound to bind to a human β2-adrenergic receptor or CCM can be analyzed prior to actual synthesis using computer modeling techniques. Only those candidates that are indicated by computer modeling to bind the target with sufficient binding energy (i.e., binding energy corresponding to a dissociation constant with the target on the order of 10⁻² M or tighter) can be synthesized and tested for their ability to bind to a human β2-adrenergic receptor or CCM using binding assays or functional assays known to those of skill in the art. The computational evaluation step thus avoids the unnecessary synthesis of compounds that are unlikely to bind the human β2-adrenergic receptor or the CCM portion of the human β2-adrenergic receptor or the CCM portion of another GPCR with adequate affinity.

A human β2-adrenergic receptor or CCM pharmacophore or candidate compound can be computationally evaluated and designed by means of a series of steps in which chemical entities or fragments are screened and selected for their ability to associate with individual binding target sites on a human β2-adrenergic receptor or CCM portion of a GPCR, including, but not limited to the CCM portion of a human β2-adrenergic receptor. One skilled in the art can use one of several methods to screen chemical entities or fragments for their ability to associate with a human β2-adrenergic receptor or CCM portion of a GPCR, and more particularly with target sites on a human 132-adrenergic receptor or CCM portion of this or another GPCR. The process can begin by visual inspection of, for example a target site on a computer screen, based on a human β2-adrenergic receptor or CCM co-ordinates, or a subset of those co-ordinates, as set forth in Appendix I. Selected fragments or chemical entities can then be positioned in a variety of orientations or “docked” within a target site of a human 132-adrenergic receptor or CCM as defined from analysis of the crystal structure data. Docking can be accomplished using software such as Quanta (Molecular Simulations, Inc., San Diego, Calif.) and Sybyl (Tripos, Inc. St. Louis, Mo.) followed by energy minimization and molecular dynamics with standard molecular mechanics forcefields such as CHARMM (Molecular Simulations, Inc., San Diego, Calif.), ICM (Molsoft, San Diego, Calif.), and AMBER (University of California, San Francisco).

Specialized computer programs can also assist in the process of selecting fragments or chemical entities. These include but are not limited to: GRID (Goodford, P. J., “A Computational Procedure for Determining Energetically Favorable Binding Sites on Biologically Important Macromolecules,” J. Med. Chem., 28, pp. 849 857 (1985)); GRID is available from Oxford University, Oxford, UK; MCSS (Miranker, A. and M. Karplus, “Functionality Maps of Binding Sites: A Multiple Copy Simultaneous Search Method,” Proteins: Structure, Function and Genetics, 11, pp. 29 34 (1991)); MCSS is available from Molecular Simulations, Inc., San Diego, Calif.; AUTODOCK (Goodsell, D. S, and A. J. Olsen, “Automated Docking of Substrates to Proteins by Simulated Annealing,” Proteins: Structure, Function, and Genetics, 8, pp. 195 202 (1990)); AUTODOCK is available from Scripps Research Institute, La Jolla, Calif.; DOCK (Kuntz, I. D., et al. “A Geometric Approach to Macromolecule-Ligand Interactions,” J. Mol. Biol., 161, pp. 269 288 (1982)); DOCK is available from University of California, San Francisco, Calif.; CERIUS II (available from Molecular Simulations, Inc., San Diego, Calif.); and Flexx (Raret, et al. J. Mol. Biol. 261, pp. 470 489 (1996)).

After selecting suitable chemical entities or fragments, they can be assembled into a single compound. Assembly can proceed by visual inspection of the relationship of the fragments to each other on a three-dimensional image of the fragments in relation to a human β2-adrenergic receptor or CCM portion of this or another GPCR receptor structure or portion thereof displayed on a computer screen. Visual inspection can be followed by manual model building using software such as the Quanta or Sybyl programs described above.

Software programs also can be used to aid one skilled in the art in connecting the individual chemical entities or fragments. These include, but are not limited to CAVEAT (Bartlett, P. A., et al. “CAVEAT: A Program to Facilitate the Structure-Derived Design of Biologically Active Molecules” In “Molecular Recognition in Chemical and Biological Problems,” Special Publ, Royal Chem. Soc., 78, pp. 182 196 (1989)); CAVEAT is available from the University of California, Berkeley, Calif.; 3D Database systems such as MACCS-3D (MDL Information Systems, San Leandro, Calif.); this area is reviewed in Martin, Y. C., “3D Database Searching in Drug Design,” J. Med. Chem., 35:2145 2154 (1992)); and HOOK (available from Molecular Simulations Inc., San Diego, Calif.).

As an alternative to building candidate pharmacophores or candidate compounds up from individual fragments or chemical entities, they can be designed de novo using the structure of a human β2-adrenergic receptor or CCM target site or the structure of a CCM target site of another GPCR, optionally, including information from co-factor(s) or known activators or inhibitor(s) that bind to the target site. De novo design can be implemented by programs including, but not limited to LUDI (Bohm, H. J., “The Computer Program LUDI: A New Method for the De Novo Design of Enzyme Inhibitors,” J. Comp. Aid. Molec. Design, 6, pp. 61 78 (1992)); LUDI is available from Molecular Simulations, Inc., San Diego, Calif.; LEGEND (Nishibata, Y., and Itai, A., Tetrahedron 47, p. 8985 (1991); LEGEND is available from Molecular Simulations, San Diego, Calif.; and LeapFrog (available from Tripos Associates, St. Louis, Mo.).

The functional effects of known human β2-adrenergic receptor or CCM ligands also can be altered through the use of the molecular modeling and design techniques described herein. This can be carried out by docking the structure of the known ligand on a human 132-adrenergic receptor or CCM model structure and modifying the shape and charge distribution of the ligand or CCM model structure to optimize the binding interactions with a human β2-adrenergic receptor or CCM. The modified structure can be synthesized or obtained from a library of compounds and tested for its binding affinity and/or effect on ribosome function. Of course, where the crystal structure of a complex between a human β2-adrenergic receptor, CCM, or subunit and a ligand is known, comparisons between said complex and the structures of the present invention can be made to gain additional information about alterations in human β2-adrenergic receptor or CCM conformation that occur upon ligand binding. This information can be used in design of optimized ligands. Compounds that interfere or activate human β2-adrenergic receptor or CCM function are especially well suited for the docking, co-crystallization, and optimization applications of the present invention.

Additional molecular modeling techniques also can be employed in accordance with the invention. See, e.g., Cohen, N. C., et al. “Molecular Modeling Software and Methods for Medicinal Chemistry,” J. Med. Chem., 33, pp. 883 894 (1990); Hubbard, Roderick E., “Can drugs be designed?” Curr. Opin. Biotechnol. 8, pp. 696 700 (1997); and Afshar, et al. “Structure-Based and Combinatorial Search for New RNA-Binding Drugs,” Curr. Opin. Biotechnol. 10, pp. 59 63 (1999).

Following pharmacophore or candidate compound design or selection according to any of the above methods or other methods known to one skilled in the art, the efficiency with which a candidate compound falling within the pharmacophore definition binds to a human β2-adrenergic receptor or CCM portion thereof or to another GPCR or CCM portion thereof can be tested and optimized using computational evaluation. A candidate compound can be optimized, e.g., so that in its bound state it would preferably lack repulsive electrostatic interaction with the target site. These repulsive electrostatic interactions include repulsive charge-charge, dipole-dipole, and charge-dipole interactions. It is preferred that the sum of all electrostatic interactions between the candidate compound and the human β2-adrenergic receptor or CCM portion thereof or other GPCR or CCM portion thereof (collectively “target”) when the candidate compound is bound to the target make a neutral or favorable contribution to the binding enthalpy or free energy.

Specific computer software is available in the art to evaluate compound deformation energy and electrostatic interactions. Examples of programs designed for such uses include, but are not limited to Gaussian 92, revision C (Frisch, M. J., Gaussian, Inc., Pittsburgh, Pa. (1992)); AMBER, version 4.0 (Kollman, P. A., University of California at San Francisco, (1994)); QUANTA/CHARMM (Molecular Simulations, Inc., San Diego, Calif. (1994)); and Insight II/Discover (Biosym Technologies Inc., San Diego, Calif. (1994)). These programs can be run, using, e.g., a Silicon Graphics workstation, Indigo, 02-R10000 or IBM RISC/6000 workstation model 550. Other hardware and software combinations can be used to carry out the above described functions, and are known to those of skill in the art.

Once a pharmacophore or candidate compound has been optimally selected or designed, as described above, substitutions can then be made in some of its atoms or side groups to improve or modify its binding properties. Generally, initial substitutions are conservative in that the replacement group will have approximately the same size, shape, hydrophobicity and charge as the original group. Components known in the art to alter conformation should be avoided in making substitutions. Substituted candidates can be analyzed for efficiency of fit to a human 132-adrenergic receptor or CCM using the same methods described above.

Assays

Any one of a number of assays of function known to those of skill in the art can be used to determine the biological activity of candidate compounds.

Candidate compound interaction with a human β2-adrenergic receptor or CCM portion thereof or to another GPCR or CCM portion thereof can be evaluated using direct binding assays including filter binding assays, such as are known to those skilled in the art. Binding assays can be modified to evaluate candidate compounds that competitively inhibit the binding of, e.g., known human β2-adrenergic receptor or CCM binding compounds. These and other assays are described in International Publication WO 00/69391, the entire disclosure of which is incorporated by reference in its entirety for all purposes. Methods of assaying for modulators of ligand binding and signal transduction include in vitro ligand binding assays using GPCRs, such as human β2-adrenergic receptor, portions thereof such as the extracellular domain, or chimeric proteins comprising one or more domains of a GPCR, oocyte GPCR expression or tissue culture cell GPCR expression, either naturally occurring or recombinant; membrane expression of a GPCR, either naturally occurring or recombinant; tissue expression of a GPCR; expression of a GPCR in a transgenic animal, etc.

GPCRs and their alleles and polymorphic variants are G-protein coupled receptors that participate in signal transduction and are associated with cellular function in a variety of cells, e.g., neurons, immune system cells, kidney, liver, colon, adipose, and other cells. The activity of GPCR polypeptides can be assessed using a variety of in vitro and in vivo assays to determine functional, chemical, and physical effects, e.g., measuring ligand binding, (e.g., radioactive ligand binding), second messengers (e.g., cAMP, cGMP, IP₃, DAG, or Ca²⁺), ion flux, phosphorylation levels, transcription levels, neurotransmitter levels, and the like. Such assays can be used to test for inhibitors and activators of a GPCR. In particular, the assays can be used to test for compounds that modulate natural ligand-induced GPCR activity, for example, by modulating the binding of the natural ligand to the receptor and/or by modulating the ability of the natural ligand to activate the receptor. Typically in such assays, the test compound is contacted with the GPCR in the presence of the natural ligand. The natural ligand can be added to the assay before, after, or concurrently with the test compound. The results of the assay, for example, the level of binding, calcium mobilization, etc. is then compared to the level in a control assay that comprises the GPCR and natural ligand in the absence of the test compound.

Screening assays of the invention are used to identify modulators that can be used as therapeutic agents, e.g., antagonists of GPCR activity.

The effects of test compounds upon the function of the GPCR polypeptides can be measured by examining any of the parameters described above. Any suitable physiological change that affects GPCR activity can be used to assess the influence of a test compound on the GPCRs and natural ligand-mediated GPCR activity. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP₃ or cAMP.

For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu Rev. Biochem. 67:653-92 (1998).

Modulators of GPCR activity are tested using GPCR polypeptides, either recombinant or naturally occurring. The protein can be isolated, expressed in a cell, expressed in a membrane derived from a cell, expressed in tissue or in an animal, either recombinant or naturally occurring. For example, neurons, cells of the immune system, adipocytes, kidney cells, transformed cells, or membranes can be used. Modulation is tested using one of the in vitro or in vivo assays described herein or others as generally known in the art. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

Ligand binding to a GPCR, a CCM, or chimeric protein can be tested in a number of formats. For example, binding can be performed in solution, in a bilayer membrane, attached to a solid phase, in a lipid monolayer, or in vesicles. Typically, in an assay of the invention, the binding of the natural ligand to its receptor is measured in the presence of a candidate modulator. Alternatively, the binding of the candidate modulator can be measured in the presence of the natural ligand. Often, competitive assay that measure the ability of a compound to compete with binding of the natural ligand to the receptor are used. Binding can be measured by assessing GPCR activity or by other assays: binding can be tested by measuring e.g., changes in spectroscopic characteristics (e.g., fluorescence, absorbance, refractive index), hydrodynamic (e.g., shape) changes, or changes in chromatographic or solubility properties.

Receptor-G-protein interactions can also be used to assay for modulators. For example, in the absence of GTP, binding of an activator such as the natural ligand will lead to the formation of a tight complex of a G protein (all three subunits) with the receptor. This complex can be detected in a variety of ways, as noted above. Such an assay can be modified to search for inhibitors. For example, the ligand can be added to the receptor and G protein in the absence of GTP to form a tight complex. Inhibitors can be identified by looking at dissociation of the receptor-G protein complex. In the presence of GTP, release of the alpha subunit of the G protein from the other two G protein subunits serves as a criterion of activation.

An activated or inhibited G-protein will in turn alter the properties of downstream effectors such as proteins, enzymes, and channels. The classic examples are the activation of cGMP phosphodiesterase by transducin in the visual system, adenylate cyclase by the stimulatory G-protein, phospholipase C by G_{q} and other cognate G proteins, and modulation of diverse channels by Gi and other G proteins. Downstream consequences such as generation of diacyl glycerol and IP₃ by phospholipase C, and in turn, for calcium mobilization e.g., by IP₃ can also be examined. Thus, modulators can be evaluated for the ability to stimulate or inhibit ligand-mediated downstream effects. In other examples, the ability of a modulator to activate a GPCR expressed in adipocytes in comparison to the ability of a natural ligand, can be determined using assays such as lipolysis (see, e.g., WO01/61359).

Activated GPCRs become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G-proteins. The kinase/arrestin pathway plays a key role in the desensitization of many GPCR receptors. Modulators can therefore also be identified using assays involving beta-arrestin recruitment. Beta-arrestin serves as a regulatory protein that is distributed throughout the cytoplasm in unactivated cells. Ligand binding to an appropriate GPCR is associated with redistribution of beta-arrestin from the cytoplasm to the cell surface, where it associates with the GPCR. Thus, receptor activation and the effect of candidate modulators on ligand-induced receptor activation, can be assessed by monitoring beta-arrestin recruitment to the cell surface. This is frequently performed by transfecting a labeled beta-arrestin fusion protein (e.g., beta-arrestin-green fluorescent protein (GFP)) into cells and monitoring its distribution using confocal microscopy (see, e.g., Groarke et al., J. Biol. Chem. 274(33):23263-69 (1999)).

Receptor internalization assays can also be used to assess receptor function. Upon ligand binding, the G-protein coupled receptor—ligand complex is internalized from the plasma membrane by a clathrin-coated vesicular endocytic process; internalization motifs on the receptors bind to adaptor protein complexes and mediate the recruitment of the activated receptors into clathrin-coated pits and vesicles. Because only activated receptors are internalized, it is possible to detect ligand-receptor binding by determining the amount of internalized receptor. In one assay format, cells are transiently transfected with radiolabeled receptor and incubated for an appropriate period of time to allow for ligand binding and receptor internalization. Thereafter, surface-bound radioactivity is removed by washing with an acid solution, the cells are solubilized, and the amount of internalized radioactivity is calculated as a percentage of ligand binding. See, e.g., Vrecl et al., Mol. Endocrinol. 12:1818-29 (1988) and Conway et al., J. Cell Physiol. 189(3):341-55 (2001). In addition, receptor internalization approaches have allowed real-time optical measurements of GPCR interactions with other cellular components in living cells (see, e.g., Barak et al., Mol. Pharmacol. 51(2)177-84 (1997)). Modulators can be identified by comparing receptor internalization levels in control cells and cells contacted with candidate compounds. For example, candidate modulators are assayed by examining their effects on receptor internalization upon binding of the natural ligand, cholesterol to its cognate receptor, i.e. the CCM of a GPCR.

Another technology that can be used to evaluate GPCR-protein interactions in living cells involves bioluminescence resonance energy transfer (BRET). A detailed discussion regarding BRET can be found in Kroeger et al., J. Biol. Chem., 276(16):12736-43 (2001).

Receptor-stimulated guanosine 5′-O-(.gamma.-Thio)-Triphosphate ([35S]GTP.gamma.S) binding to G-proteins can also be used as an assay for evaluating modulators of GPCRs. [³⁵S]GTPγS is a radiolabeled GTP analog that has a high affinity for all types of G-proteins, is available with a high specific activity and, although unstable in the unbound form, is not hydrolyzed when bound to the G-protein. Thus, it is possible to quantitatively assess ligand-bound receptor by comparing stimulated versus unstimulated [³⁵S]GTP.gamma.S binding utilizing, for example, a liquid scintillation counter. Inhibitors of the receptor-ligand interactions would result in decreased [³⁵S]GTPγS binding. Descriptions of [³⁵S]GTPγS binding assays are provided in Traynor and Nahorski, Mol. Pharmacol. 47(4):848-54 (1995) and Bohn et al., Nature 408:720-23 (2000).

The ability of modulators to affect ligand-induced ion flux can also be determined. Ion flux can be assessed by determining changes in polarization (i.e., electrical potential) of the cell or membrane expressing a GPCR. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., Pflügers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., J. Pharmacol. Meth. 25:185-193 (1991); Holevinsky et al., J. Membrane Biology 137:59-70 (1994)). Generally, the compounds to be tested are present in the range from 1 pM to 100 mM.

Preferred assays for G-protein coupled receptors include cells that are loaded with ion or voltage sensitive dyes to report receptor activity. Assays for determining activity of such receptors can also use known agonists and antagonists for other G-protein coupled receptors and the natural ligands disclosed herein as negative or positive controls to assess activity of tested compounds. In assays for identifying modulatory compounds (e.g., agonists, antagonists), changes in the level of ions in the cytoplasm or membrane voltage are monitored using an ion sensitive or membrane voltage fluorescent indicator, respectively. Among the ion-sensitive indicators and voltage probes that can be employed are those disclosed in the Molecular Probes 1997 Catalog. For G-protein coupled receptors, promiscuous G-proteins such as Gα15 and Gα16 can be used in the assay of choice (Wilkie et al., Proc. Nat'l Acad. Sci. USA 88:10049-10053 (1991)). Such promiscuous G-proteins allow coupling of a wide range of receptors to signal transduction pathways in heterologous cells.

Receptor activation by ligand binding typically initiates subsequent intracellular events, e.g., increases in second messengers such as IP₃, which releases intracellular stores of calcium ions. Activation of some G-protein coupled receptors stimulates the formation of inositol triphosphate (IP₃) through phospholipase C-mediated hydrolysis of phosphatidylinositol (Berridge & Irvine, Nature 312:315-21 (1984)). IP₃ in turn stimulates the release of intracellular calcium ion stores. Thus, a change in cytoplasmic calcium ion levels, or a change in second messenger levels such as IP₃ can be used to assess G-protein coupled receptor function. Cells expressing such G-protein coupled receptors can exhibit increased cytoplasmic calcium levels as a result of contribution from both intracellular stores and via activation of ion channels, in which case it can be desirable although not necessary to conduct such assays in calcium-free buffer, optionally supplemented with a chelating agent such as EGTA, to distinguish fluorescence response resulting from calcium release from internal stores.

Other assays can involve determining the activity of receptors which, when activated by ligand binding, result in a change in the level of intracellular cyclic nucleotides, e.g., cAMP or cGMP, by activating or inhibiting downstream effectors such as adenylate cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., Proc. Natl. Acad. Sci. U.S.A. 88:9868-9872 (1991) and Dhallan et al., Nature 347:184-187 (1990)). In cases where activation of the receptor results in a decrease in cyclic nucleotide levels, it can be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-gated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In one embodiment, changes in intracellular cAMP or cGMP can be measured using immunoassays. The method described in Offermanns & Simon, J. Biol. Chem. 270:15175-15180 (1995) can be used to determine the level of cAMP. Also, the method described in Felley-Bosco et al., Am. J. Resp. Cell and Mol. Biol. 11:159-164 (1994) can be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates are separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which can or can not contain an agonist).

In another embodiment, transcription levels can be measured to assess the effects of a test compound on ligand-induced signal transduction. A host cell containing the protein of interest is contacted with a test compound in the presence of the natural ligand for a sufficient time to effect any interactions, and then the level of gene expression is measured. The amount of time to effect such interactions can be empirically determined, such as by running a time course and measuring the level of transcription as a function of time. The amount of transcription can be measured by using any method known to those of skill in the art to be suitable. For example, mRNA expression of the protein of interest can be detected using northern blots or their polypeptide products can be identified using immunoassays. Alternatively, transcription based assays using reporter genes can be used as described in U.S. Pat. No. 5,436,128, herein incorporated by reference. The reporter genes can be, e.g., chloramphenicol acetyltransferase, firefly luciferase, bacterial luciferase, beta-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)).

The amount of transcription is then compared to the amount of transcription in either the same cell in the absence of the test compound, or it can be compared with the amount of transcription in a substantially identical cell that lacks the protein of interest. A substantially identical cell can be derived from the same cells from which the recombinant cell was prepared but which had not been modified by introduction of heterologous DNA. Any difference in the amount of transcription indicates that the test compound has in some manner altered the activity of the protein of interest.

Samples that are treated-with a potential GPCR inhibitor or activator are compared to control samples comprising a known ligand such as, e.g., an agonist, an antagonist, a partial agonist or an inverse agonist, without the test compound to examine the extent of modulation. Control samples (untreated with activators or inhibitors) are assigned a relative GPCR activity value of 100. Inhibition of a GPCR is achieved when the GPCR activity value relative to the control is about 90%, optionally 50%, optionally 25-0%. Activation of a GPCR is achieved when the GPCR activity value relative to the control is 110%, optionally 150%, 200-500%, or 1000-2000%.

In one embodiment the invention provides soluble assays using molecules such as a domain, e.g., a ligand binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; a GPCR; or a cell or tissue expressing a GPCR, either naturally occurring or recombinant. In another embodiment, the invention provides solid phase based in vitro assays in a high throughput format, where the domain, chimeric molecule, GPCR, or cell or tissue expressing a GPCR is attached to a solid phase substrate.

Certain screening methods involve screening for a compound that modulate the expression of the GPCRs described herein, or the levels of natural ligands, e.g., ASP and stanniocalcins. Such methods generally involve conducting cell-based assays in which test compounds are contacted with one or more cells expressing the GPCR or ligand and then detecting an increase or decrease in expression (either transcript or translation product). Such assays are typically performed with cells that express the endogenous GPCR or ligand.

Expression can be detected in a number of different ways. As described herein, the expression levels of the protein in a cell can be determined by probing the mRNA expressed in a cell with a probe that specifically hybridizes with a transcript (or complementary nucleic acid derived therefrom) of the GPCR or protein ligand. Probing can be conducted by lysing the cells and conducting Northern blots or without lysing the cells using in situ-hybridization techniques (see above). Alternatively, protein can be detected using immunological methods in which a cell lysate is probed with antibodies that specifically bind to the protein.

Other cell-based assays are reporter assays conducted with cells that do not express the protein. Certain of these assays are conducted with a heterologous nucleic acid construct that includes a promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, beta-glucuronidase, CAT (chloramphenicol acetyl transferase), luciferase, beta-galactosidase and alkaline phosphatase.

In these assays, cells harboring the reporter construct are contacted with a test compound. A test compound that either modulates the activity of the promoter by binding to it or triggers a cascade that produces a molecule that modulates the promoter causes expression of the detectable reporter. Certain other reporter assays are conducted with cells that harbor a heterologous construct that includes a transcriptional control element that activates expression of the GPCR or ligand and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

In one embodiment the invention provides soluble assays using molecules such as a domain, e.g., a ligand binding domain, an extracellular domain, a CCM, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; a GPCR; or a cell or tissue expressing a GPCR, either naturally occurring or recombinant. In another embodiment, the invention provides solid phase based in vitro assays in a high throughput format, where the domain, chimeric molecule, GPCR, or cell or tissue expressing a GPCR is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-1500 different compounds. It is possible to assay several different plates per day; assay screens for up to about 6,000-20,000 different compounds is possible using the integrated systems of the invention.

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the signal transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and are appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or hetero functional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Modulators

Inhibitors and/or activators identified according to the methods of the invention can be provided from libraries of compounds available from a number of sources or can be derived by combinatorial chemistry approaches known in the art. Such libraries include but are not limited to the available Chemical Director, Maybridge, and natural product collections. In one embodiment of the invention libraries of compounds with known or predicted structures can be docked to a human 132-adrenergic receptor or other GPCR CCM structures of the invention. In another embodiment, the libraries for a CCM can include compounds with a ring-type structure. In one aspect of this embodiment, the ring-type structure can stack into a CCM pocket through interactions involving it electron orbitals. In another embodiment, the libraries can include a linker component or moiety. In some embodiments, the linker can include from about 10-22 atoms and can include one or more of C, O, N, S, and/or H atoms. In another embodiment, the libraries can include a ligand binding site (also known as the ligand, agonist, or antagonist binding pocket) component or moiety. In some embodiments, the libraries can include drug-like molecules, i.e., molecules having structural attributes of one or more compounds known to bind to and/or affect a physiologic function of a GPCR.

In some embodiments, the invention includes compounds that can be tested as modulators of GPCR activity. Compounds tested as modulators of GPCRs can be any small chemical compound or biological entity. Typically, test compounds will be small chemical molecules and peptides. Essentially any chemical compound can be used as a potential modulator or ligand in the assays of the invention, although most often compounds can be dissolved in aqueous or organic (especially DMSO-based) solutions. The assays are designed to screen large chemical libraries by automating the assay steps. The assays are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or ligand libraries are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Russell & Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

It is noted that modulators that compete with the binding and/or activity of the known ligands for a human β2-adrenergic receptor or the CCM can be used to treat various diseases including, but not limited to, coronary artery disease, atherosclerosis, thrombosis, obesity, diabetes, stroke, and other diseases.

In one embodiment, a modulator binds to a site on a GPCR. In one aspect, the site is a CCM. In another aspect, the site is a site other than the CCM. In another aspect, the site is a ligand binding site. In another aspect, the modulator has a first moiety that binds to a CCM. In another aspect, the first moiety is connected to a linker. In another aspect, the first moiety and the linker are connected to a second moiety that binds to a site other than the CCM site. In another aspect, the first and second moieties are not connected by a linker and are both present in a composition.

Computer-Based Modeling of Membrane Proteins, GPCRs, and CCMs

Protein-ligand docking aims to employ principles by which protein receptors, e.g., a human β2AR, recognize, interact, and associate with molecular substrates and compounds to predict the structure arising from the association between a given compound and a target protein of known three-dimensional structure.

In protein-ligand docking, the search algorithm can allow the degrees of freedom of the protein-ligand system to be sampled sufficiently as to include the true binding modes. Three general categories of algorithms have been developed to address this problem of ligand flexibility: systematic methods; random or stochastic methods; and simulation methods.

Systematic search algorithms attempt to explore all degrees of freedom in a molecule. These algorithms can be further divided into three types: conformational search methods, fragmentation methods, and database methods.

In conformational search methods, all rotatable bonds in the ligand are systematically rotated through 360° using a fixed increment, until all possible combinations have been generated and evaluated. As the number of structures generated increases immensely with the number of rotatable bonds (combinatorial explosion), the application of this type of method, in its purest form, is very limited.

Fragmentation methods use two different approaches to incrementally grow the ligands into the active site. One approach is by docking the several fragments into a site, e.g., a CCM, and linking them covalently to recreate the initial ligand (“the place-and-join approach”). Another approach is by dividing the ligand into a rigid core-fragment that is docked in first place and flexible regions that are subsequently and successively added (“the incremental approach”). DOCK (see above) is an example of s docking programs that use a fragmentation search method.

Database methods using libraries of pre-generated conformations or conformational ensembles to address the combinatorial explosion problem. A example of a docking program using database methods is FLOG which generates a small set of 25 database conformations per molecule based on distance geometry, that are subsequently subject to a rigid docking protocol.

Random search algorithms sample the conformational space by performing random changes to a single ligand or a population of ligands. At each step, the alteration performed is accepted or rejected based on a predefined probability function. There are three basic types of methods based on random algorithms: Monte Carlo methods (MC), Genetic Algorithm methods (GA), and Tabu Search methods.

Simulation methods employ a rather different approach to the docking problem, based on the calculation of the solutions to Newton's equations of motion. Two major types exist: molecular dynamics (MD) and pure energy minimization methods.

Scoring functions normally employed in protein-ligand docking are generally able to predict binding free energies within 7-10 kJ/mol and can be divided into three major classes: force field-based, empirical, and knowledge-based scoring functions.

In force-field based scoring, standard force fields quantify the sum of two energies: the interaction energy between the receptor and the ligand, and the internal energy of the ligand. The energies are normally accounted through a combination of a van der Waals with an electrostatic energy terms. A Lennard-Jones potential is used to describe the van der Waals energy term, whereas the electrostatic term is given by a Coulombic formulation with a distance-dependent dielectric function that lessens the contribution from charge-charge interactions.

Empirical scoring functions are based on the idea that binding energies can be approximated by a sum of several individual uncorrelated terms. Experimentally determined binding energies and sometimes a training set of experimentally resolved receptor-ligand complexes are used to determine the coefficients for the various terms by means of a regression analysis.

Knowledge-based scoring functions focus on following the rules and general principles statistically derived that aim to reproduce experimentally determined structures, instead of binding energies, trying to implicitly capture binding effects that are difficult to model explicitly. Typically, these methods use very simple atomic interactions-pair potentials, allowing large compound databases to be efficiently screened. These potentials are based on the frequency of occurrence of different atom-atom pair contacts and other typical interactions in large datasets of protein-ligand complexes of known structure. Therefore, their derivation is dependent on the information available in limited sets of structures.

Consensus Scoring combines the information obtained from different scores to compensate for errors from individual scoring functions, therefore improving the probability of finding the correct solution. Several studies have demonstrated the success of consensus scoring methods in relation to the use individual functions schemes.

Using the Protein-ligand docking methods described above the predicted association between a given compound selected from chemical libraries, see above for examples, and the CCM included in the human β2AR structure described in the PDB file (PDB accession number 3D4S) of Appendix I can be made. These methods will therefore allow the generation of a binding profile for any known compound in the CCM of human β2AR based on the simulated docking of compounds in the CCM.

In another embodiment, a form of computer-assisted drug design is employed in which a computer system is used to generate a three-dimensional structure of the candidate GPCR based on the structural information encoded by the amino acid sequence. This will allow use of the methods described above to identify candidate compounds based on their ability to dock in the CCM of the predicted GPCR structure. In one aspect, the input amino acid sequence of the GPCR interacts directly and actively with a pre-established algorithm in a computer program to yield secondary, tertiary, and quaternary structural models of the GPCR. The models of the GPCR structure are then examined to identify the position and structure of the CCM. The position and structure of the predicted CCM is then used to identify various compounds that modulate ligand-receptor binding using the methods described above.

The three-dimensional structural model of the GPCR is generated by entering protein amino acid sequences of at least 10 amino acid residues or corresponding nucleic acid sequences encoding a GPCR polypeptide into the computer system. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the GPCR is then generated by the interaction of the amino acid sequence and the computer system, using software known to those of skill in the art. Any method of protein structure modeling such as ab-initio modeling, threading or sequence-sequence based methods of fold recognition. In one embodiment, the AS2TS system of protein structure modeling is used. In other embodiments, a sequence alignment in combination with a threshold protein sequence similarity to determine a set of protein sequences for which to model protein structure is used. In one aspect, sequence alignments are generated for the set of sequences to be modeled with sequences of proteins with solved empirical structure in a protein structure databank known to one of skill in the art. If the sequences to be modeled have a sufficient similarity to one or more sequences with known protein structure, then the three dimensional structure of the sequence can be modeled.

The amino acid sequence represents a primary structure that encodes the information necessary to form the secondary, tertiary and quaternary structure of the GPCR of interest. In one embodiment, software can look at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

In another embodiment, protein structure alignments can be used to determine the structure of GPCRs using a known structure of β2AR (Appendix I). Protein structure alignments preferably are sets of correspondences between spatial co-ordinates of sets of carbon alpha atoms which form the ‘backbone’ of the three-dimensional structure of polypeptides, although alignments of other backbone or side chain atoms also can be envisioned. These correspondences are generated by computationally aligning or superimposing two sets of atoms order to minimize distance between the two sets of carbon alpha atoms. The root mean square deviation (RMSD) of all the corresponding carbon alpha atoms in the backbone is commonly used as a quantitative measure of the quality of alignment. Another quantitative measure of alignment is the number of equivalent or structurally aligned residues.

In another embodiment, a GPCR structure is calculated based on a solved structure of a β2AR by computationally aligning or superimposing two sets of atoms to minimize distance between the two sets of carbon alpha atoms (i.e., the alpha carbon atoms of a β2AR and an unknown GPCR structure), followed by one or more of simulated annealing and energy minimization. The result of this calculation is a computed structure for a GPCR that provides atomic co-ordinates for the alpha carbon backbone as well as side chain atoms.

A variety of methods for generating an optimal set of correspondences can be used in the present invention. Some methods use the calculation of distance matrices to generate an optimal alignment. Other methods maximize the number of equivalent residues while RMSD is kept close to a constant value.

In the calculation of correspondences, various cutoff values can be specified to increase or decrease the stringency of the alignment. These cutoffs can be specified using distance in Angstroms. Depending on the level of stringency employed in the present invention, the distance cutoff used is less than 10 Angstroms or less than 5 Angstroms, or less than 4 Angstroms, or less than 3 Angstroms. One of ordinary skill will recognize that the utility of stringency criterion depends on the resolution of the structure determination.

In another embodiment of the present invention, the set of residue-residue correspondences are created using a local-global alignment (LGA), as described in US Patent Publication Number 2004/0185486. In this method, a set of local superpositions are created in order to detect regions which are most similar. The LGA scoring function has two components, LCS (longest continuous segments) and GDT (global distance test), established for the detection of regions of local and global structure similarities between proteins. In comparing two protein structures, the LCS procedure is able to localize and superimpose the longest segments of residues that can fit under a selected RMSD cutoff. The GDT algorithm is designed to complement evaluations made with LCS searching for the largest (not necessary continuous) set of ‘equivalent’ residues that deviate by no more than a specified distance cutoff.

Using the protein structure alignments described above, the structure of β2AR in Appendix I can be used as a model on which to discern the structure of other GPCRs and/or their predicted CCMs, e.g. such as disclosed in Appendices II-XI.

Once the GPCR structure has been generated, the CCM regions are identified by the computer system. Computational models seek to identify the CCM by characterization of the three dimensional structure of the GPCR.

Some methods of CCM identification use triangulation such as weighted Delaunay triangulation to determine pocket volumes (castP). Other methods use spheres to determining protein pocket volumes (Q-site-finder, UniquePocket).

Conserved CCM identification seeks to identify conserved CCMs through associating the residues which form CCMs with residues which form a conserved CCM in homologous protein sequences or structures, e.g., see Appendix I.

One method of identifying CCMs entails filling the three dimensional protein structures with spheres, creating a “negative image” of the structure. A cutoff distance, such as 8 Angstroms, is used to determine spheres which interact with residues. Spheres are labeled as conserved or not-conserved based on their interaction with residues which form a conserved CCM. The conserved spheres are clustered based on their three dimensional co-ordinates to identify a set of spheres with interact with conserved residues and are proximal in three dimensional space forming a cluster. Three-dimensional structures for potential compounds are generated by entering chemical formulas of compounds. The three-dimensional structure of the potential compound is then compared to that of the GPCR protein CCM to identify compounds that bind to GPCR CCM. Binding affinity between the GPCR CCM and compound is determined using energy terms to determine which ligands have an enhanced probability of binding to the protein.

The following examples are set forth so that the invention can be understood more fully. The examples are for illustrative purposes only and are not to be construed as limiting this invention in any manner.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols. A and B(1992).

Methods

Expression and Purification

β₂AR(E122W)-T4L was identical to β₂AR-T4L in all but two features. (1) Residue 122 was mutated from a glutamate to a tryptophan using standard site-directed mutagenesis protocols, and (2) the C-terminus was further truncated to residue 348 eliminating a total of 65 residues. High-titer recombinant baculovirus was obtained using standard protocols in the Bac-to-Bac system (Invitrogen) and used to infect spodoptera frugiperda (Sf9) insect cells at a multiplicity of infection of five and the expression was allowed to proceed for 48 hours. Insect cell membranes were initially disrupted by nitrogen cavitation in a hypotonic buffer containing 10 mM Hepes pH 7.5, 20 mM KCl and 10 mM MgCl₂. Extensive washing of the membranes was carried out by repeated centrifugation and Dounce homogenization to strip the membranes of soluble and membrane associated proteins. Membranes were flash frozen and stored at −80° C. until further use. Prior to solubilization, prepared membranes were thawed on ice in the presence of 1 mM timolol, 2 mg/mL iodoacetamide, and protease inhibitors. Membranes were then solubilized by incubation in the presence of 0.5% w/v dodecylmaltoside (DDM) for 2-4 hours at 4° C. After solubilization, the solution was clarified at 100,000 g and the resulting supernatant incubated with Co²⁺ charged TALON IMAC resin overnight at 4° C. The resin was washed with 20 mM imidazole to remove impurities followed by an elution of the receptor with 200 mM imidazole. An additional IMAC step after desalting was used to concentrate and deglycosylate (PNGase) the receptor (increasing purity up to 98%). The protein was maintained in 20 mM Hepes pH 7.5, 150 mM NaCl, 0.05% DDM, 0.01% cholesteryl hemisuccinate (CHS) and 1 mM timolol throughout the purification unless otherwise indicated. Timolol concentration was increased to 5 mM on the second IMAC column to avoid ligand depletion in the subsequent concentration step which utilized a 600 μL Vivaspin cartridge with a 100 kDa molecular weight cutoff

Crystallization

Crystals were generated from a 10% w/w cholesterol/monoolein lipidic cubic phase after reconstituting the protein from a 30 mg/mL solution and overlaying the lipid phase with a solution of 28% w/v PEG 400, 300 mM K Formate, 100 mM Bis-tris propane pH 7.0 and 2 mM timolol. Crystals were obtained from 25 mL of cubic phase and harvested directly from glass sandwich plates in which they were grown (Cherezov et al., 2004).

Thermal Stability Assay

Thermal stability data was collected by a modified procedure utilizing a thiol-reactive coumarin maleimide, 7-diethylamino-3-(4′-maleimidylphenyl)-4-methylcoumarin (CPM). The data were collected at 35° C. in the presence of guanidinium hydrochloride (GnHCL) at three different concentrations. The chemical denaturant was included to speed the process of unfolding thereby minimizing the effects of dehydration on data processing error. Protein was purified as according to standard procedure until the final Ni-NTA purification and concentration step at which point timolol and cholesteryl hemisuccinate (CHS) were washed from the protein with five column volumes of buffer. The protein was then eluted at approximately 2 mg/mL as a 10× stock. The protein was then diluted 3-fold into a 50 mM Hepes pH 7.5, 150 mM NaCl buffer containing 2 μg of CPM dye (diluted from a 4 mg/mL stock in dimethyl formamide) and incubated at 4° C. for 10 minutes. Various combinations of timolol and CHS in dodecylmaltoside (DDM) were then added to the mixture at 2× final concentration and allowed to equilibrate for 30 minutes at 4° C. before the final dilution to working volume with the appropriate concentration of GnHCl in the assay buffer. Data was collected as soon as possible after the addition of the chemical denaturant to reduce the degree of unfolding prior to measurement. Fluorescence of the CPM dye was measured on a Tecan Genios Pro fluorescent plate reader using 340 nM excitation filter with a 35 nM bandpass and a 475 nM emission filter with a 20 nM bandpass. Readings were measured every minute for three hours. The data were fit to a single exponential decay curve using GraphPad Prism Software (San Diego, Calif.).

Lipidic Cubic Phase Crystallization

For lipidic cubic phase (LCP) crystallization, robotic trials were performed using an in meso crystallization robot (Cherezov et al., 2004). Glass sandwich plates in 96-well format (Cherezov and Caffrey, 2003; Cherezov et al., 2004) were filled with 25 or 50 mL protein-laden LCP drops overlaid by 0.8 μL of precipitant solution in each well and sealed with a glass coverslip. All operations starting from mixing lipid and protein were performed at room temperature (˜21-23° C.). Crystals were obtained in 28% (v/v) PEG 400, 0.3 M potassium formate, 0.1 M Bis-tris propane pH 7.0 and 2 mM timolol using 10% (w/w) cholesterol in monoolein as the host lipid.

Data Collection and Structure Solution

X-ray data were collected on the 231D-B/D beamline (GM/CA CAT) at the Advanced Photon Source, Argonne, Ill. using a 10 μm minibeam (wavelength 1.0332 Å) and a MarMosaic 300 CCD detector. A complete dataset was collected from a single crystal at 5 Å resolution using 5× attenuated beam, 5 s exposure and 1° oscillation per frame. However, some crystals diffracted to a maximum of 2.6 Å resolution upon 5 s exposure with 1× attenuated beam, therefore, we collected 10-20° wedges of high-resolution data from more than 20 crystals, 10 of which were combined and scaled against the lower resolution dataset to obtain a 94% complete 2.8 Å data using the software program XDS (Kabsch, 1993). Initial phases for ^{tim}β₂AR(E122W)-T4L were obtained by molecular replacement using the receptor and T4L portion of β₂AR-T4L (PDBID: 2RH1) separately with the program Phaser (McCoy et al., 2005). Refinement was carried out using the Phenix software suite (Adams et al., 2002) followed iteratively by model rebuilding in Coot utilizing 2Fₒ-F_{c} sigma-A weighted maps, as well as 2Fₒ-F_{c} composite omit maps calculated using the Bhat procedure (Bhat, 1988).

Ligand Binding Assays

Saturation isotherm: Cell pellets (cultivated in the presence and absence of cholesterol-β-methylcyclodextrin) were suspended in ice-cold 25 mM Hepes, pH 7.4 lysis buffer, containing protease inhibitors (Complete protease inhibitor cocktail tablet, Roche Applied Science, USA) and homogenized with 30 strokes using a Dounce homogenizer. Cellular debris and DNA were removed by centrifugation at 400×g for 10 minutes at 4° C., and the supernatants were collected. Crude plasma membranes were isolated by centrifugation of the supernatants at 150,000×g for 60 minutes at 4° C., and crude plasma membranes were further washed three times by repeat centrifugation and resuspension in 25 mM Hepes, 800 mM NaCl, pH 7.4, and containing protease inhibitors. Prior to the ligand binding assays, the membrane pellets were resuspended in ligand binding buffer (TME: 50 mM Tris-HCl, 10 mM MgCl₂, 0.5 mM EDTA, pH 7.4). The samples were tested for binding with Levo-[Ring, Propyl-³H(N)]-Dihydroalprenolol Hydrochloride [³H]DHA, (81 Ci/mmol, Perkin-Elmer Life Sciences, USA) and ³H-CGP-12177 (4-(3-t-butylamino-2-hydroxypropoxy)-[5,7-³H]benzimidazole-2-one hydrochloride, (49 Ci/mmol, Perkin-Elmer Life Sciences, USA). Crude plasma membranes (0.2-1 μg of total protein per reaction) were incubated for 45 minutes at room temperature with serial dilutions of the radioligand (0.05-10 nM). Incubations were rapidly terminated by filtration using a Tomtec Mach III cell harvester (Tomtec) through a 96-well GF/B filter plate (MultiScreen Harvest plate, Millipore Corp.), and rinsed five times with 500 μl of ice-cold buffer (50 mM Tris-HCl, pH 7.4). The harvest plates were dried, and 30 μl of OptiPhase “HiSafe” III scintillation liquid (Perkin-Elmer Life Sciences) were added. The bound radioactivity was measured using a Packard's TopCounter NTX. Nonspecific binding was determined in parallel reactions in the presence of an excess of Alprenolol (100 μM, Sigma-Aldrich, USA), and specific binding was defined as the difference between total and nonspecific binding. Protein concentrations were determined with the BCA protein assay (Pierce, USA), using bovine serum albumin as a reference. All incubations were performed in triplicates, and independent experiments were repeated at least two times. Equilibrium dissociation constants (K_{d}) and maximal receptor levels (Bₘₐₓ) were calculated from the results of saturation experiments using GraphPad Prism Software.

Competition Binding Assays: Competitive ligand binding was assayed by incubating isolated crude plasma membranes (1.5 μg of protein per reaction) with [³H]DHA and competing ligands in TME for 45 minutes at room temperature. Reactions were terminated and the harvest plates were processed as above. Nonspecific binding was determined in the presence of an excess of Alprenolol (100 μM). The radioligand concentrations were close to equilibrium dissociation constants (K_{d}˜4.5 nM) assayed above. Ligands tested in competitive binding assays were Timolol (Sigma) and Isoproterenol hydrochloride (Tocris, Bioscience) (16 concentrations of each). The apparent affinities (apparent Kᵢ) of each ligand at each receptor were determined by using nonlinear regression analysis (GraphPad Prism), applying the Cheng-Prusoff equation (Kᵢ={IC50/[(L/Kd)+1]} and assuming one-site binding. Incorporation of the cholesterol into crude plasma membranes was verified using the Amplex-Red cholesterol assay kit (Invitrogen, Molecular Probes).

### Example 1

### Crystal Generation and Structure Solution

High-level expression of β₂AR(E122W)-T4L was carried out in Sf9 insect cells using standard protocols. A simplified purification scheme was enabled by the presence of the E122W mutation, which resulted in a higher yield of functionally active receptor in the folded state than for the wild-type (85% for E122W vs. 25% for wild-type), as judged by size-exclusion chromatography using a fluorescent-labeled alprenolol probe and traditional binding assays (Roth et al., 2008). This shift to a functionally folded receptor eliminates the need for a ligand affinity chromatography step in the purification. In addition, the high expression levels of 2 mg of receptor per liter of cell culture enabled a single metal-affinity chromatography step to achieve greater than 90% homogeneity, thus mitigating the effects of delipidation on the final purified protein. The β₂AR(E122W)-T4L was purified and crystallized in the presence of a saturating concentration of S-(−)-1-(t-Butylamino)-3-[(4-morpholino-1,2,5-thiadiazol-3-yl)oxyl]-2-propanol maleate (timolol) (Table 1).

TABLE 1

^{tim}β₂AR(E122W)-T4L

Data collection (APS GM/CA CAT ID-B/D, 10 μm beam)

| | |
|---|---|
| Space group | P2₁2₁2₁ |
| a, b, c (Å) | 40.0, 75.7, 172.7 |
| No. of reflections processed | 54,405 |
| No. unique reflections | 12,782 |
| Resolution (Å) | 50-2.8 (3.0-2.8) |
| R_{sym} | 14.3 (56.9) |
| Mean I/σ(I) | 6.9 (1.9) |
| Completeness (%) | 94.0 (91.0) |
| Redundancy | 4.2 (2.9) |
| Refinement | |
| Resolution (Å) | 20-2.8 (3.0-2.8) |
| No. reflections (test set) | 12782 (640) |
| R_{work}/R_{free} | 23.1 (30.2)/27.3 (38.3) |
| No. atoms | 3700 |
| Protein | 3528 |
| Ions, lipids, ligand and other | 152 |
| Water | 20 |
| Overall B-values (Å²) | 69 |
| β₂AR | 63 |
| T4-Lysozyme | 80 |
| Ligand | 49 |
| Cholesterol #1 | 71 |
| Cholesterol #2 | 84 |
| Lipid | 71 |
| R.m.s deviations | |
| Bond lengths (Å) | 0.010 |
| Bond angles (°) | 1.313 |
| Ramachandran plot statistics (%): | |
| Most favored regions | 98.2 |
| Additionally allowed regions | 1.8 |
| Disallowed regions | 0 |

*Highest resolution shell is shown in parenthesis.
R_{sym} = Σₕₖₗ |I(hkl) − <I(hkl)>|/Σₕₖₗ(hkl), where <I(hkl)> is the mean of the symmetry equivalent reflections of I(hkl).

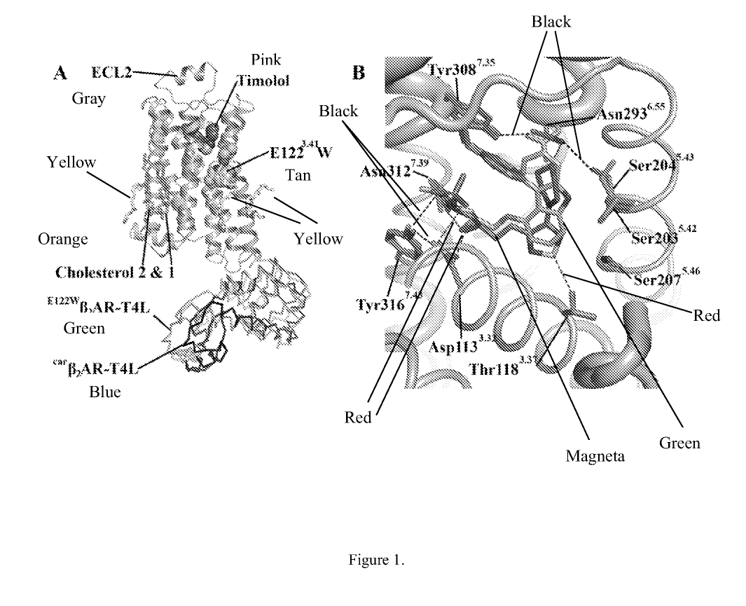

Comparison between timolol bound β₂AR(E122W)-T4L (^{tim}β₂AR(E122W)-T4L) and carazolol bound ^{car}β₂AR-T4L (PDB ID: 2RH1) (Overall RMSD: 1.8 Å, receptor RMSD: 0.34 Å) reveals a change in the relative tilt angle between T4L and the receptor, as well as a few minor shifts in ICL2 and ECL3 that can be explained by the monomers packing in a higher symmetry space group with different crystal packing interactions (FIG. 1A). In addition, the relative orientation between the two domains of T4L has shifted significantly so that when domain 1 (residues 2-11, 59-161) is aligned between the two structures (RMSD=0.6 Å), domain 2 (residues 12-28) has an RMSD of 5.1 Å. However, as indicated, the receptor portion of the fusion protein is very similar between the two crystal structures. As such it is difficult to explain the occurrence of the altered space group as the conditions used for crystallization of each species are very similar. Thus it is necessary to examine the differences in the protein and in its preparation. There are three possibilities for the observed shift in intermolecular interactions. The stabilizing mutant is altering the packing environment either through loss of a buried charge or through increased interactions with the monoolein lipid membrane. This possibility is supported by the presence of electron density for a lipid species adjacent to the W122^{3.41} modeled as monoolein in the current structure. No corresponding density was seen in this region for the ^{car}β₂AR-T4L structure despite higher resolution data. A second related possibility is the presence of endogenous insect plasma membrane lipid being retained through the shortened purification protocol. The final possibility is the shortened length of the C-terminus in the current structure enables an altered crystal packing environment despite the fact that the same number of residues are ordered at the C-terminus in both structures. Given the current information it is difficult to distinguish between these three possibilities although the first two seem more plausible as the majority of the interreceptor packing interactions occur within the lipid membrane.

### Example 2

### Timolol Binding Interactions

Given the established functional similarities between β₂AR and β₂AR(E122W) (Roth et al., 2008), as well as between β₂AR and β₂AR-T4L (Rosenbaum et al., 2007), we feel confident in drawing relevant conclusions from studies focusing on the molecular interactions associated with β₂AR and its small molecule effectors using the β₂AR(E122W)-T4L system, which, as shown here, is structurally equivalent to ^{car}β₂AR-T4L. The binding orientation of timolol is similar to that of carazolol, where the oxypropanolamine tail forms strong interactions with the polar triad (Asp113^{3.32}, Asn312^{7.39} and Trp316^{7.43}), and the morpholino-thiadiazole head group binds in a similar orientation to the carbazole head group of carazolol (FIG. 1B). However, two subtle yet relevant differences occur between the carazolol and timolol binding modes. The thiadiazole ring of timolol binds deeper into the receptor pocket allowing an additional hydrogen bonding interaction with Thr118^{3.37}. Secondly, an additional polar interaction is formed between the ether moiety of the morpholino ring of timolol and Asn293^{6.55}, which has been implicated in agonist binding and the transfer of conformational information to the G protein coupling interface (Hannawacker et al., 2002; Wieland et al., 1996). Due to hydrogen bonding between Asn293^{6.55} and Tyr308^{7.35}, both residues have a slightly altered side-chain rotamers relative to ^{car}β₂AR-T4L to accommodate the receptor-timolol interaction (FIG. 1B). This interaction can result in timolol being a more effective inverse agonist due to interactions formed with helix VI that restrict the receptor from forming an active conformation. Although a direct comparison of inverse agonism between timolol and carazolol has not been reported, a study describing the suppression of basal activity of β₂AR by various ligands resulted in 28% suppression by timolol and partial enhancement of signaling by carvedilol, a chemically homologous ligand to carazolol (Baker et al., 2003). Another study of the suppression of basal activity of β₂AR indicated a much stronger inverse agonism effect of timolol (Chidiac et al., 1994) and carazolol itself suppressed 50% basal activity in a reconstituted liposome system (Rasmussen et al., 2007). These data indicate that the magnitude of inverse agonism is context dependent, although one would expect the rank order of inverse agonist efficacy to remain constant.

### Example 3

### The β₂AR Cholesterol Binding Site

Over the past few years, studies highlighting the effect of cholesterol depletion on ligand binding characteristics of a few receptors in membranes have been reported and recently reviewed (Pucadyil and Chattopadhyay, 2006). In addition, the thermal stability of both the oxytocin receptor and the β₂AR is improved in the presence of cholesterol and cholesteryl hemisuccinate (CHS), respectively (Gimpl and Fahrenholz, 2002; Yao and Kobilka, 2005). For the oxytocin receptor, cholesterol or cholesterol analogues that enhance thermal stability also shift the receptor to the high-affinity agonist binding state implying allosteric modulation by cholesterol.

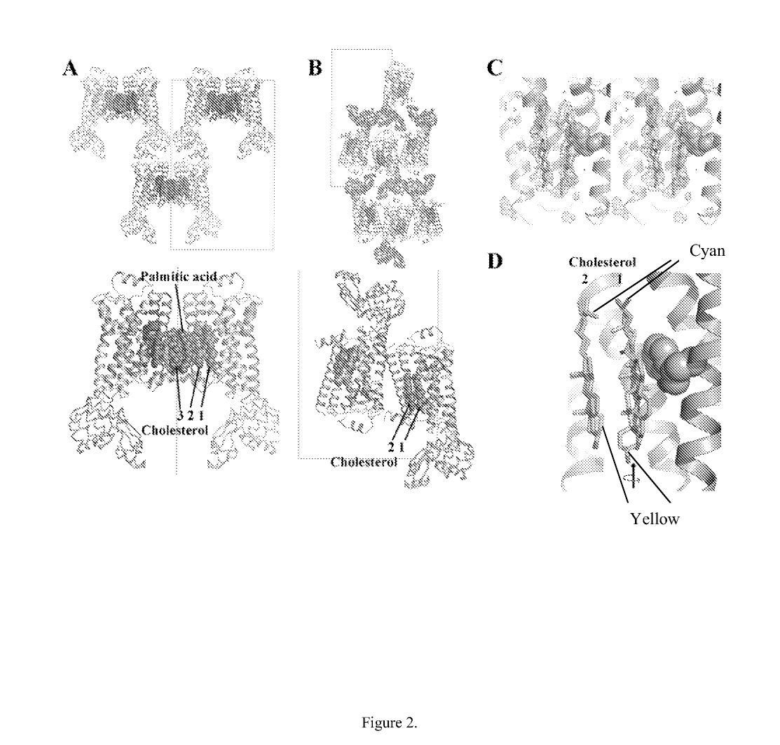

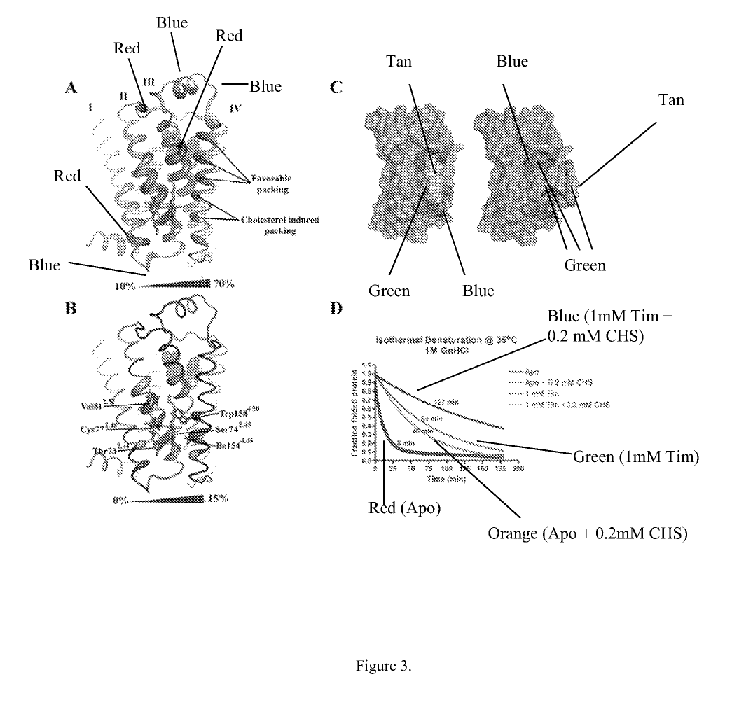

The structure of ^{car}β₂AR-T4L had interpretable density for three molecules of cholesterol per monomer of protein with visible density for the palmitate moiety that is post-translationally attached to Cys341 and located between cholesterol molecules 2 and 3 of a symmetry-related monomer (FIG. 2A). Due to the position of the cholesterol molecules relative to the crystal-packing interface, the biological relevance of the binding sites could not be decoupled from potential crystal packing artifacts (FIG. 2A) and the orientation of the sterol ring relative to the receptor could not be established. With the generation of crystals for ^{tim}β₂AR(E122W)-T4L in a higher symmetry space group and the antiparallel orientation of the receptor molecules within the asymmetric unit, the role of cholesterol in crystal packing is reduced (FIG. 2B). The retention of binding sites for cholesterol 1 and 2 in the current structure indicate that two sites are not experimental artifacts and that cholesterol binding in this region can be physiologically relevant. The electron density for cholesterol 1 in the ^{tim}β₂AR(E122W)-T4L does not support the position seen in the ^{car}β₂AR-T4L structure, rather a 90° rotation and slight translation along the long axis of the molecule are necessary for optimal fitting of the experimental electron density (FIGS. 2C and 2D). This alternate orientation results in the cholesterol molecules packing with the sterol ring system of cholesterol 2 related to cholesterol 1 by an approximate two-fold rotation and a slight translational shift along an axis parallel to the ring system. Both cholesterol molecules bind in a shallow surface groove formed by segments of helices I, II, III and IV and, thus, provide an increase in the intramolecular occluded surface area, a parameter linked to the enhanced stability of proteins from thermophilic organisms and used to compare the internal packing of helices in membrane proteins relative to soluble protein (DeDecker et al., 1996; Eilers et al., 2002; Eilers et al., 2000). The occluded surface area method was used to analyze packing value (PV) differences between the transmembrane helices compared to each other and to their equivalent helices in rhodopsin (PDBID: 1U19) (Pattabiraman et al., 1995). It is noted that overall the rhodopsin helical bundle has slightly tighter packing interactions than β₂AR (PV: 0.43 vs. 0.42), perhaps reflective of its greater stability. In the absence of cholesterol, helix IV has the loosest packing of all the transmembrane helices in β₂AR (PV without cholesterol: 0.37), whereas it is tightly packed in rhodopsin (PV: 0.45). Binding interactions with cholesterol 1 increase the packing for this helix (PV with cholesterol: 0.39), which is indicative of a decrease in mobility (FIG. 3A, B). Both cholesterol molecules contribute equally to the increase in packing of helix II upon cholesterol binding (FIG. 3A, B). However, in the absence of cholesterol, helix II has the second highest occluded surface area of the transmembrane helices so that the marginal gain from cholesterol interactions on this helix would not significantly affect the mobility of the helix or the overall thermal stability of the protein. Cholesterol increases the packing interactions for both helix II and IV explaining structurally the observed increase in thermal stability, however helix IV is still unable to achieve the high degree of intramolecular packing interactions associated with rhodopsin, indicating that this helix is perhaps one of the weakest points in the β₂AR fold. While the overall effect on normalized occluded surface area upon cholesterol binding appears small, the differences between rhodopsin and β₂AR over the 7TM bundle are also very small even though the resulting change in thermostability appears quite significant. Similarly, comparison of hyperthermophilic proteins to their mesophilic counterparts indicates an increase in normalized occluded surface area of 4% at the most, putting the change in packing and its relationship to stability on a more absolute scale (Eilers et al., 2002). It is likely that the main effect of cholesterol on stability is mediated through its effect on helix IV where cholesterol 1 serves as a bridge to helix II, which forms many intramolecular interactions contributing to the core of the receptor fold (FIGS. 3B and 3C).

The thermal stability of β₂AR(E122W)-T4L solubilized in the detergent dodecylmaltoside (DDM) in the presence and absence of CHS and timolol was further characterized by monitoring the extent of covalent coupling of receptor cysteines to the thiol-reactive dye, 7-diethylamino-3-(4′-maleimidylphenyl)-4-methylcoumarin (CPM), as a function of time and guanidinium hydrochloride (GnHCl) concentration (FIG. 3D) (Alexandrov et al., 2008). The half-life of thermal denaturation of β₂AR(E122W)-T4L at 35° C. changes significantly upon incorporation of both timolol and CHS (FIG. 3D). The overall stabilizing effect of CHS in 1 M GnHCl results in a 5-fold increase in the half-life of protein denaturation in the absence of timolol and a 2-fold increase over protein bound to timolol alone. Relative to the apo protein, with a half-life of 8 minutes, incorporation of both ligands simultaneously increases the half-life to 127 minutes, which is almost a 16-fold improvement in stability (FIG. 3D).

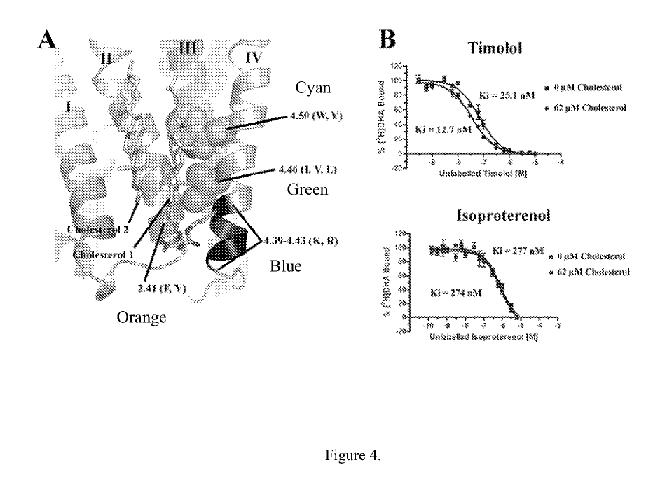

The specific interaction of other membrane proteins with cholesterol has previously been postulated and a consensus sequence for cholesterol binding has been proposed within the context of the peripheral-type benzodiazepine receptor (Li and Papadopoulos, 1998). Cholesterol binding appears to be dependent on the presence of an incipient cleft located at a membrane interfacial region, which contains at least one aromatic residue and a positively charged residue capable of participating in electrostatic interactions with apical hydroxyl group (Epand et al., 2006; Jamin et al., 2005). The cleft formed by β₂AR helices I, II, III and IV is capable of accommodating two cholesterol molecules although there are a reduced number of interactions between β₂AR and cholesterol 2. Interactions with helix IV are perhaps most analogous to the previously defined cholesterol binding motif and together with an additional site on helix II form a receptor cholesterol consensus motif (CCM) defined by four spatially distributed interactions with cholesterol 1. The aromatic Trp158^{4.50} is almost universally conserved (94%) among class A GPCRs and appears to contribute the most significant interaction with the sterol ring of cholesterol 1 through a CH-π interaction and the edge of ring D. The hydrophobic Ile154^{4.46} interacts with rings A and B and is 60% homology conserved (35% by identity). An aromatic residue from helix II, Tyr70^{2.41} in β₂AR, forms Van der Waals interactions with ring A of cholesterol 1 and hydrogen bonds to Arg151^{4.43}. Importantly, a positive charge at an analogous position to Arg151^{4.43} is only 22% conserved with either arginine or lysine occupying the position. However, due to the non-specific nature of electrostatic interactions in the interfacial region of the membrane, nearby positions with positive charge might also serve the role of interacting with the cholesterol hydroxyl group, the limits of which will need to be established structurally. Consideration of the spatial distribution of conserved residues that are important for cholesterol binding in β₂AR allow the definition of the CCM for cholesterol interaction within the class A receptors based on the Ballesteros-Weinstein numbering scheme as follows: [4.39-4.43(R,K)]---[4.50(W,Y)]---[4.46(I,V,L)]---[2.41(F,Y)] where 26% of class A receptors (21% for human class A receptors) (Table 2) are predicted to bind cholesterol at the same site as β₂AR (FIG. 4A). The four positions within the CCM are listed according to their perceived rank order of binding interactions with the positively charged position contributing the most binding energy and the aromatic residue on helix II contributing the least.

TABLE 2
List of receptors with the CCM and the less restrictive rCCM motif

| | |
|---|---|
| CCM | rCCM |

| | | | |
|---|---|---|---|
| HTR1A | DRD3 | HTR5A | LGR7 |
| HTR1B | DRD1 | ADRA2A | MTNR1A |
| HTR1E | EDG1 | ADRA2B | MTNR1B |
| HTR1F | EDG2 | ADRA2C | GPR50 |
| HTR2A | EDG3 | ADORA1 | MC1R |
| HTR2B | GPR10 | ADRB1 | MTLR1 |
| HTR2C | GPR19 | ADRB3 | NPFF1 |
| HTR4 | GPR21 | CNR2 | NPGPR |
| HTR6 | GPR52 | CCKAR | TACR3L |
| HTR7 | MC3R | DRD4 | NTSR2 |
| ADRA1A | MC4R | DRD5 | NPY1R |
| ADRA1B | MC5R | EDNRB | OR10H1 |
| ADRA1D | TACR1 | FPR1 | OR10H2 |
| ADORA2A | TACR2 | GALR1 | OR10H3 |
| ADORA3 | TACR3 | GALR2 | OR10J1 |
| CHRM1 | NTSR1 | GALR3 | OR11A1 |
| CHRM2 | NPY2R | CCKBR | OPN4 |
| CHRM3 | OPN1SW | GHSR | LTB4R |
| CHRM4 | OPN1MW | GPR45 | PTGER3 |
| CHRM5 | OPN1LW | GPR63 | PTGER4 |
| MC2R | HCRTR1 | GPR72 | PTGFR |
| ADRB2 | HCRTR2 | GPR1 | TBXA2R |
| DRD2 | OXTR | GPR3 | TRHR |
| | | GNRHR | AVPR1A |
| | | HRH1 | |
| | | HRH2 | |

In β₂AR, addition of cholesterol induces an increase in the affinity for the partial inverse agonist timolol, but no change is observed for the full agonist isoproterenol (FIG. 4B). While the physiological effect of cholesterol binding to β₂AR is unknown it has been established that β₂AR preferentially sequesters to cholesterol rich caveolae in neonatal rat cardiomyocyte cultures (Ostrom et al., 2001; Rybin et al., 2000; Steinberg, 2004), and partitions out of the caveolae upon stimulation (Rybin et al., 2000). While this compartmentalization has been shown to be due in part to the presence of a C-terminal PDZ binding domain that directs the trafficking of β₂AR to caveolae (Xiang and Kobilka, 2003), receptor cholesterol interactions might also play a role in defining the trafficking properties of β₂AR and other GPCRs. Furthermore sequence disparities in the cholesterol binding site among receptors might potentiate the trafficking effect of cholesterol binding, analogous to the trafficking and cholesterol sequestration effects observed for caveolin and other cholesterol binding proteins (Epand et al., 2005).

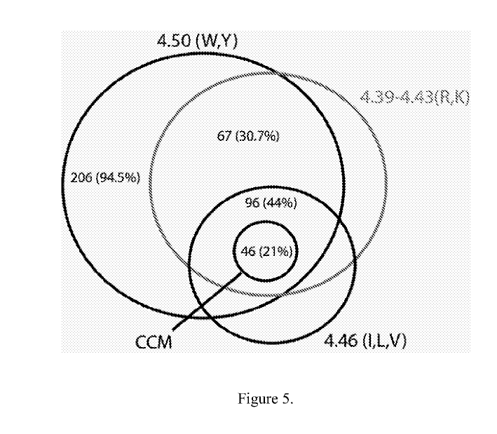

The presence of an aromatic residue at position 2.41 is the most restrictive of the four rules and also appears to be the least important for binding of cholesterol based on the structure. In the absence of this requirement 44% of human class A receptors would contain the revised CCM (rCCM) and should bind cholesterol in a similar manner as β₂AR (FIG. 5; Table 2). Mutagenesis studies can illuminate the actual rank order importance of these positions in cholesterol binding, it is increasingly apparent that sterols play an important direct role GPCR stability and pharmacology. The accession numbers of the proteins throughout this description and listed in Table 2 can be derived from the NCBI database (National Center for Biotechnology Information) maintained by the National Institute of Health, U.S.A. The accession numbers and associated sequence information are as provided in the database on Jun. 9, 2008. One of ordinary skill can use the abbreviated gene names listed in Table 2 to search the NCBI website and determine the gene's mRNA and protein sequences. In another aspect, one of ordinary skill can use the abbreviated gene names in Table 2 to search the HUGO database (HUGO website; HGNC Search tool) and determine the gene's mRNA and protein sequences, full unabbreviated gene name(s), and synonym(s).

### Example 4

### Development of a Pharmacophore Model Using the CCM

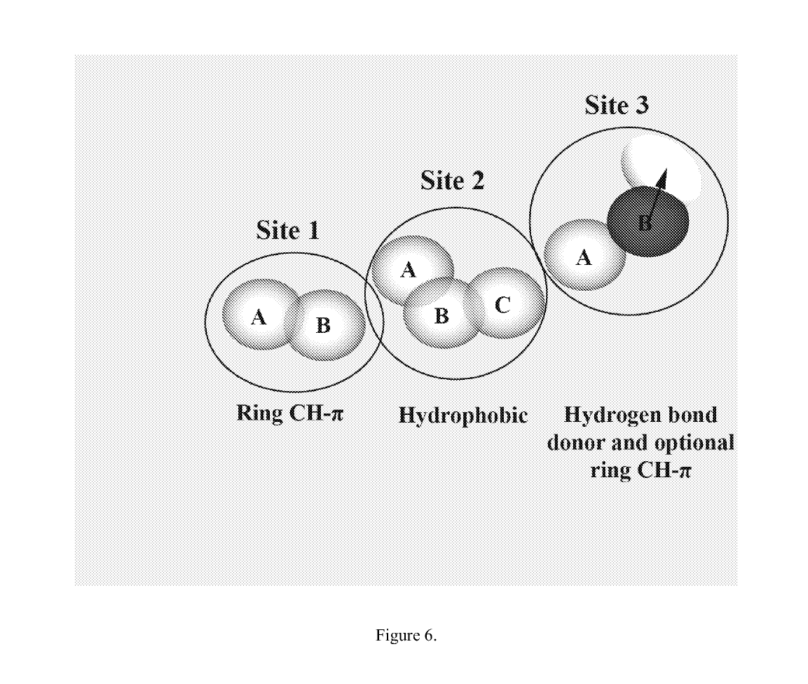

A ligand-based pharmacophore model was developed using the structure predicted interactions between cholesterol and the CCM of β₂AR (FIG. 6). Site 1 contains two interactions points (A and B) with Trp158^{4.50} through CH-π electron hydrogen bonding interactions. The strain associated with the sterol ring structure can induce a more polarizable CH bond, which can participate in hydrogen bonding interactions under favorable conditions. The low dielectric constant where the interactions are located can serve to strengthen these bonds. Alternatively, Site 1 can contain an aromatic group which can interact with the tryptophan residue through ring edge interactions. Site 2 contains a cluster of three hydrophobic groups that can participate in non-directional interactions. Satisfaction of this site can require the presence of at least one hydrophobic moiety at either of the three points: A, B, or C. Site 3 contains an optional polar CH bond in proximity to a hydrogen bond donor such as the hydroxyl group of cholesterol.

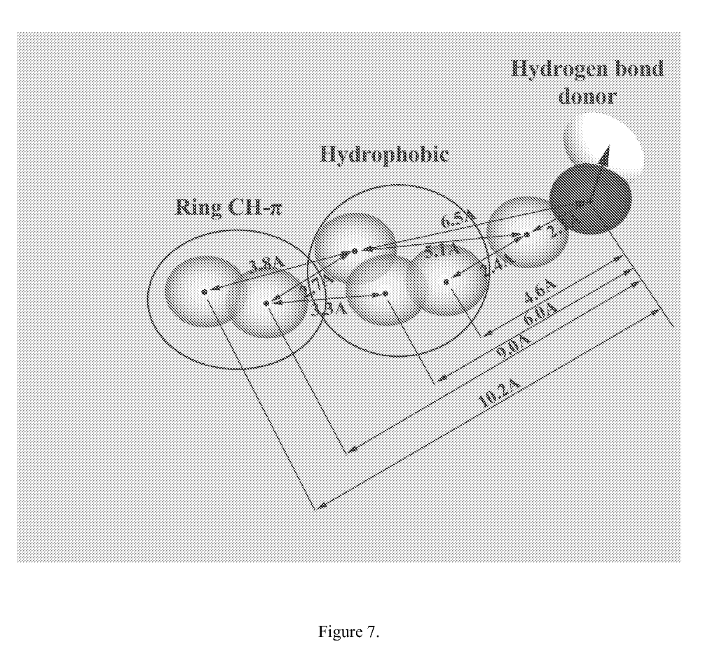

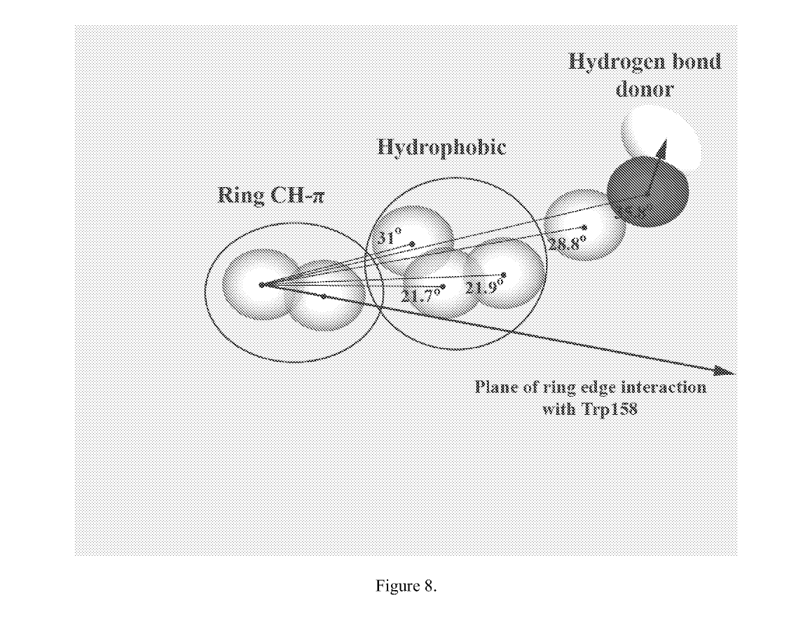

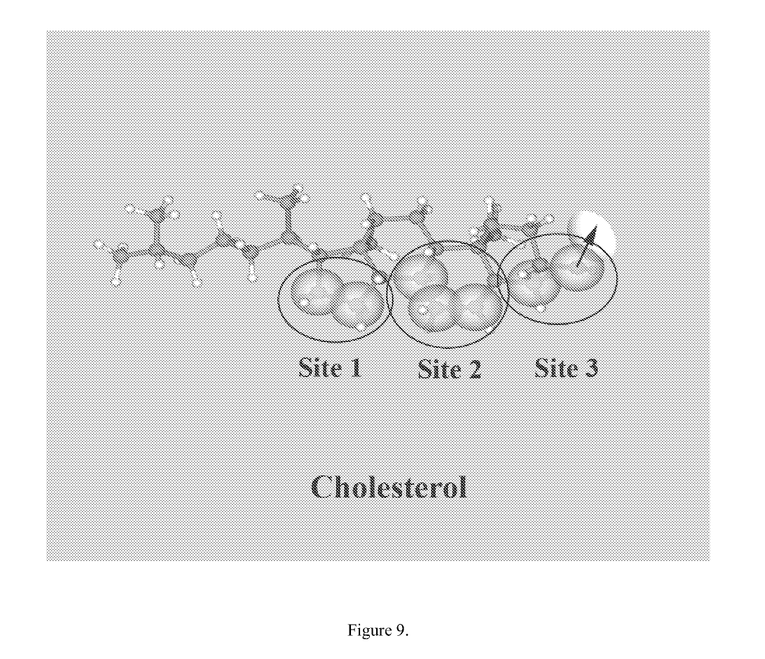

FIG. 7 shows the distance constraints between select regions of the sites and points of the pharmacophore model. FIG. 8 shows the angle constraints between select regions of the sites of the pharmacophore model. Each angle is represented by the angle defined by Site 1A (far left sphere of Site 1) and the line projected along the direction of the ring edge interactions associated with Site 1. FIG. 9 shows the mapping of cholesterol onto the pharmacophore model demonstrating the applicability of the model to compound development using methods known to one of ordinary skill in the art. Using methods known to one of ordinary skill in the art a number of compounds possessing a wide variety of steroid-like ring structures are predicted to bind to the CCM, including but not limited to the compounds listed in Table 3.

TABLE 3

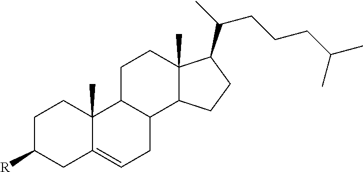

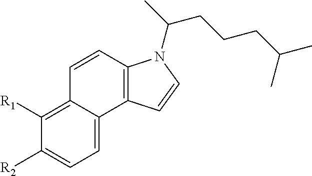

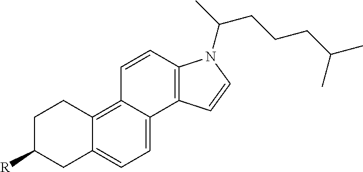

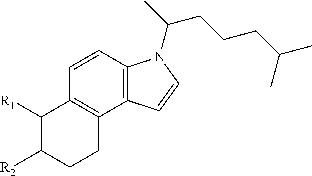

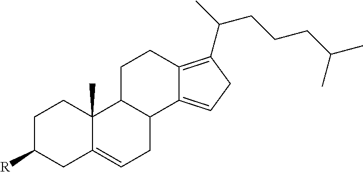

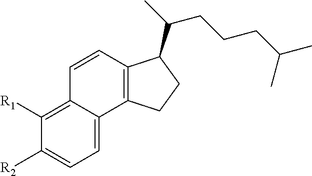

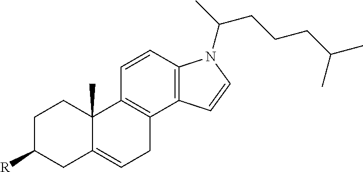

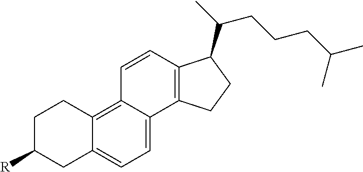

R = H
Acetate
Aldehyde
Benzoate
Caproate
Carboxylate
Chloro
Cyano
Dichloroacetate
Ethoxycarbonyl
Ethyl ester
Ethyleneketal
Formate
Hemisuccinate
Hydrazone
Oxime
Phenylpropionate
Proprionate
Sulphate

In addition, non-steroid based compounds can have some degree of binding interaction with the CCM, especially if an aromatic group is available for ring edge interactions with the tryptophan quadrapole. Examples of receptors for which known non-steroidal compounds are predicted to have some degree of binding interaction with their CCM are listed in Table 4.

TABLE 4
List of receptors that have a CCM, as grouped by drug development
stage.

| Approved drug on market | Ongoing clinical trials |
|---|---|
| Serotonin | Neuropeptide |
| Adrenergic | Cannabinoid |
| Muscarinic | Endothelin |
| Dopamine | Galanin |
| NK1 | Leukotriene B4 |
| Oxytocin | Motilin |
| Gonadotropin releasing | |
| hormone | |
| Vasopressin | |
| Thromboxane | |
| Histamine | |
| Thyrotropin | |

In addition, the pharmacophore model can be used for the development of long-acting β₂AR agonists by coupling ligands that bind to the receptors orthosteric binding site with new compounds capable of binding to residues within the CCM. This approach is generally applicable to many human class A GPCRs as 44% of human class A GPCRs possess a form of the CCM (see Table 2 above) and would be amenable to bifunctional ligand development.

### Example 5

### Modeling of Salmeterol on the β₂AR Structure

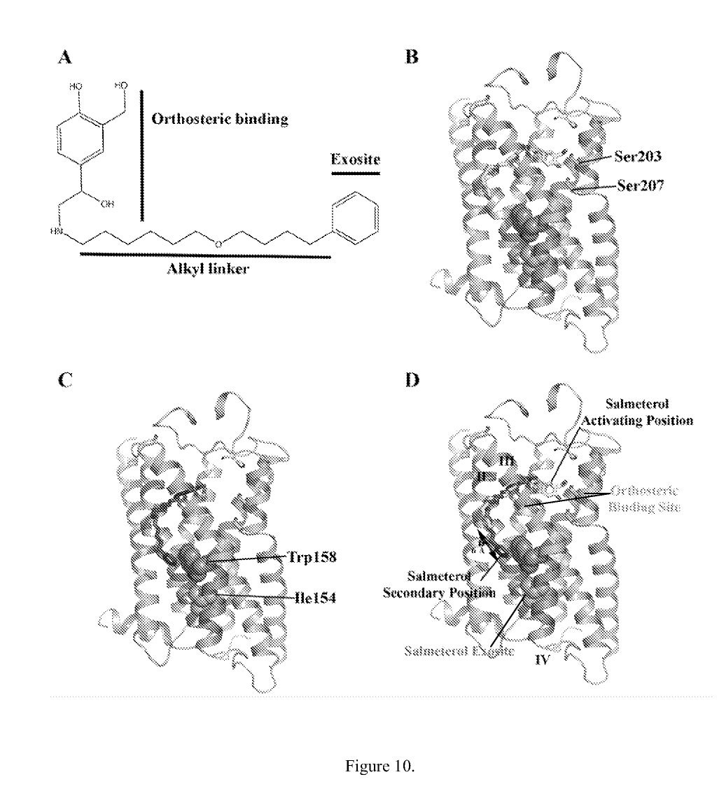

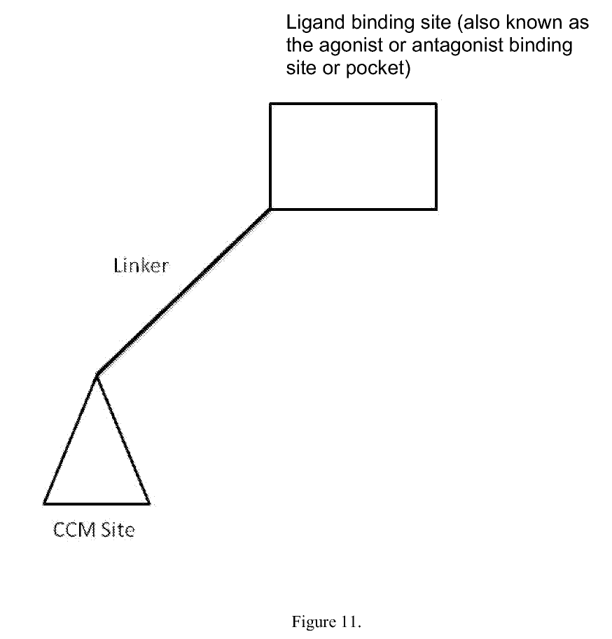

The agonist salmeterol is known to bind to the active sites of β₂AR and induce sustained activation (Green and Liggett, 1996). In addition, Green et al. provide evidence supporting the existence of an exosite binding mode for salmeterol in addition to the canonical orthosteric binding site, suggesting that residues in the CCM participate in the exosite binding of the phenyl group of this ligand. Thus, the clues from the development of salmeterol can be applied to the compound design efforts of the present invention in a more direct fashion. For instance, modeling of salmeterol into the β₂AR structure using the established constraints of known interaction points in both the orthosteric binding site and exosite allows visualization of the orientation of this ligand, providing insight alterations that can be made to optimize candidate compounds (FIG. 10 and accompanying legend). FIG. 10 shows that linker length can be optimized to allow the most direct path from the orthosteric site to the CCM exosite without building in excess hydrophobicity. In addition, to connect the two sites one end of the bifunctional ligand can insert between two helices, most likely helices II and III. For this insertion to occur there can either be e.g., significant structural flexibility in the helical orientations or the head group of either end must be quite small. As an increase in structural flexibility is often associated with an agonist bound receptor, known agonist orthosteric head groups in applicable receptors are linked to the CCM exosite (Table 4). In addition, other small functional groups and compounds are designed that still interact optimally with the CCM based on the pharmacophore model, see above, and docking studies based on compounds such as those in Table 3 and Table 4. Based on this modeling study and the pharmacophore design study above, FIG. 11 shows a general diagram scheme of a CCM site binding moiety linked by a linker to a ligand binding site binding moiety.

### Example 6

### Binding of Salmeterol to Multiple Sites on Human β₂AR

Recombinant human β₂AR is purchased from commercial sources or is prepared using recombinant baculovirus-infected insect cells using standard methods well known to one skilled in the art. β₂AR is diluted in assay buffer and salmeterol (dissolved in an aqueous or organic solvent) is added. The β₂AR and salmeterol are allowed to interact at 18-37° C. Typically step is performed in a volume of 50 μl (range 10-200 ul). Binding of salmeterol to the active site and the CCM of β₂AR is detected using assays and methods known in the art or described in detail above.

### Example 7

### The Calculated Structure of the GPCR TACR1 Based on the Empirically-Determined Structure for Human β₂AR

The structure of the GPCR TACR1 (SEQ ID NO: 3) was calculated based on the empirically determined structure for β2AR in Appendix I. Sequences of the proteins were obtained from ExPASy (see ExPASy web-site and proteomics server available from the Swiss Institute for Bioinformatics (SIB)) and aligned using CLUSTALW (see above). The model of the structure was generated using the program MODELLER (available at the web-site for Andrej Sali; University of California, San Francisco) and the β2AR structure as a template. Residues not modeled on the N and C termini and 3^{rd} intracellular loop were deleted. The resulting structure was minimized using CNS (Crystallography & NMR System; see Brunger et al., Crystallography & NMR system: A new software suite for macromolecular structure determination; Acta Crystallogr D Biol Crystallogr.; 1998 Sep. 1; 54(Pt 5):905-21; Yale University; New Haven, Conn.). Initial minimization of the side chains was performed while holding the backbone atoms fixed. Secondary minimization of the entire model was performed until convergence. The PDB file for the resulting calculated structure is shown in Appendix II. The residues important for ligand binding to the GPCR are shown in Table 5 using the Ballesteros-Weinstein numbering scheme described above.

TABLE 5

| Ballesteros | | | | | | | | | | |
|---|---|---|---|---|---|---|---|---|---|---|
| index | HTR1A | ADRA1A | ADORA2a | CHRM1 | MC2R | DRD2 | EDG1 | TACR1 | NTSR1 | OXTR |
| 1.39 | | | E | | | | | | Y | |
| 1.42 | | | | | | | | | L | |
| 1.46 | | | | | | | I | | G | |
| 2.57 | | | | | | | Y | N | | |
| 2.61 | | | | | | | | N | | Q |
| 2.65 | | | | | | | G | | | |
| 3.25 | | | | | D | | | | | |
| 3.28 | | | | | I | | R | | | V |
| 3.29 | | | | | D | | E | | | |
| 3.32 | D | D | | D | | D | | | | |
| 3.36 | | | T | | | | L | | | M |
| 3.40 | | | | | I | | | | | |
| 4.56 | | | | | T | | | | | |
| 4.60 | | | | | | | | | | |
| 5.38 | | | | | | | | | | Y |
| 5.42 | S | S | | T | | S | | | | |
| 5.43 | T | A | | A | | S | | | | |
| 5.46 | A | S | | A | | S | | | | |
| 6.44 | F | F | | F | | F | F | | | F |
| 6.51 | F | F | | Y | F | F | | | Y | F |
| 6.52 | F | F | | N | | F | | | | F |
| 6.54 | | | | | | | | | R | |
| 6.55 | | | N | | | | | | R | |
| 7.35 | | | | | F | | | Y | Y | |
| 7.42 | | | S | | | | | | | |
| 7.43 | Y | Y | H | Y | | Y | | | Y | |

Ligands that bind the ligand binding sites of the classes of GPCRs of Tables 2 and 5 are shown in Table 6.

TABLE 6

Serotonin
5-Carboxamidotryptamine, [1,2-3H]-
8-Hydroxy-DPAT, [Propyl-2,3-ring-1,2,3-3H]-
BRL-43694, [9-Methyl-3H]-
Carboxamidotryptamine, [1,2-3H]-5-
DOI, [125I]-(±)-
GR 65630, [N-Methyl-3H]-
Hydroxytryptamine Binoxalate, 5-[2-14C]-
Hydroxytryptamine Creatinine Sulfate, 5-[1,2-3H(N)]-
Ketanserin Hydrochloride (R41 468), [Ethylene-3H]-
Lysergic Acid Diethylamide, [N-Methyl-3H]-
Lysergic Acid Diethylamide, 2-[125I]iodo-(+)
MPPF, [Methyl-3H]-
Muscarinic
AF-DX 384, [2,3-dipropylamino-3H]-
Pirenzepine, [N-Methyl-3H]-
Quinuclidinyl Benzilate, L-[Benzilic-4,4′-3H]-
Neurokinin
Eledoisin, [125I]-(Lys4)-
Kallidin (Des Arg10, Leu9), [3,4-Prolyl-3,4-3H(N)]-
Neurokinin A, [125I]-Substance K-
Neurokinin B, [125I]His, MePhe7-
Senktide, [Phenylalanyl-3,4,5-3H]-
SR 48968, [Benzamide-4-3H]-
Substance P, [125I]Tyr8]-
Substance P, [Leucyl-3,4,5-3H(N)]-,
Substance P, [125I]-(Lys3)-
Substance-P (9-Sar, 11-Met(O2)), [2-Prolyl-3,4-3H]-
GNRH
Human Chorionic Gonadotropin, ([125I]-HCG)-
GnRH, [125I]-(His5,D-Tyr6)-
LH-RH, [125I]-[D-Trp6]
Luteinizing Hormone Releasing Hormone, [125I]Tyr5-
Thromboxane
SQ29,548, [3H]-
Thyrotropin
Thyrotropin Releasing Hormone (3-methyl-histidine2),
[L-histidyl-4-3H(N), L-propyl-3,4-3H(N)]-
Thyrotropin Releasing Hormone, [125I]-(His2)-
Cannabinoid
CP 55940, [Side Chain-2,3,4-3H(N)]-
WIN 55212-2, [5,7-Naphthyl-3H]-
Galanin
Galanin (Human), [125I]-
Motilin
Motilin, [125I]-
Adrenergic
(−)-CGP-12177, [5,7-3H]-
(−)Iodocyanopindolol [125I]-
(−)Iodopindolol [125I]-
(±)-β-([125I]Iodo-4-hydroxyphenyl)-ethyl-aminomethyl-tetralone
Dihydroalprenolol Hydrochloride, Levo-[Ring, Propyl-3H(N)]-
Epinephrine, Levo-[N-methyl-3H]-
Iodoclonidine, p-[125I]-(2-[(2,6-dichloro-4-[125I]-
MK-912, [Methyl-3H]-
Norepinephrine Hydrochloride, DL-[7-3H(N)]-
Norepinephrine, Levo-[7-3H]-
Norepinephrine, Levo-[ring-2,5,6-3H]-
Prazosin, [7-Methoxy-3H]-
Propranolol, L-[4-3H]-
Rauwolscine, [Methyl-3H]-
UK-14,304, [Imidazolyl-4,5-3H]-
Yohimbine, [Methyl-3H]-
Dopamine
Dihydroxyphenylethylamine, 3,4-[7-3H]-
Dihydroxyphenylethylamine, 3,4-[Ring-2,5,6-3H]-
Iodospiperone, 2′-[125I]-
Methylspiperone, [N-Methyl-3H]-
Quinpirole, [N-Propyl-3H]-
R-(+)-trans-7-hydroxy-PIPAT, [125I]
Raclopride, [Methoxy-3H]-
SCH 23390, [N-Methyl-3H]-
Spiperone, [Benzene ring-3H]-
Sulpiride, (−), [methoxy-3H]-
YM-09151-2, [N-Methyl-3H]-
Oxytocin
Oxytocin, [125I]Tyr2-
Oxytocin, [Tyrosyl-2,6-3H]-
Vasopressin
Ornithine Vasotocin Analog, [125I]-
Vasopressin (Linear), VIA Antagonist
(Phenylacetyl1, 0-Me-D-Tyr2, [125I-Arg6]-)
Vasopressin, 8-arginine, [Phenylalanyl-3,4,5-3H(N)]-
Histamine
Histamine Dihydrochloride, [Ring, Methylenes-3H(N)]-
Methylhistamine Dihydrochloride, N-a-[methyl-3H]-
Pyrilamine (Mepyramine), [pyridinyl 5-3H]-
Neuropeptide
Neuropeptide FF, [D-Tyr1[125I], N-MePhe3]-
Neuropeptide S (Human), [125I]Tyr10-
Neuropeptide W23, [125I]-
Peptide YY (Human), ([125I]-PYY)-
Peptide YY, [125I]-(Leu31, Pro34)-
Endothelin
Endothelin-1 (Human), [125I]Tyr13-
Leukotriene
LTB4, [3H]

Examples of which classes the GPCRs of Tables 2 and 5 belong are shown in Table 7.

TABLE 7

| Serotonin | Adrenergic | Muscarinic |
|---|---|---|
| HTR1A | ADRA1A | CHRM1 |
| HTR1B | ADRA1B | CHRM2 |
| HTR1E | ADRA1D | CHRM3 |
| HTR1F | ADRB2 | CHRM4 |
| HTR2A | ADRB1 | CHRM5 |
| | (rCCM) | |
| HTR2B | ADRB3 | |
| | (rCCM) | |
| HTR2C | ADRA2A | |
| | (rCCM) | |
| HTR4 | ADRA2B | |
| | (rCCM) | |
| HTR6 | ADRA2C | |
| | (rCCM) | |
| HTR7 | | |
| HTR5A (rCCM) | | |

| Dopamine | Neurokinin | Oxytocin |
|---|---|---|
| DRD2 | TACR1 | OXTR |
| DRD4 (rCCM) | TACR2 | |
| DRD5 (rCCM) | TACR3 | |

| GNRH | Vasopressin | Thromboxane |
|---|---|---|
| GNRHR | AVPR1A | TBXA2R |
| Histamine | Thyrotropin | Neuropeptide |
| HRH1 | GHSR | NPY2R |
| HRH2 | | NPY1R |
| | | (rCCM) |

| Cannabinoid | Endothelin | Galanin |
|---|---|---|
| CNR2 (rCCM) | EDNRB | GALR1 |
| | (rCCM) | (rCCM) |
| | | GALR2 (rCCM) |
| | | GALR3 (rCCM) |

| Leukotriene | Motilin |
|---|---|
| LTB4R | MTLR1 |
| (rCCM) | (rCCM) |

The residues important for binding to the CCM are shown in Table 8 using the Ballesteros-Weinstein numbering scheme described above.

TABLE 8

| Ballesteros | | | | | | | | | | |
|---|---|---|---|---|---|---|---|---|---|---|
| index | HTR1A | ADRA1A | ADORA2a | CHRM1 | MC2R | DRD2 | EDG1 | TACR1 | NTSR1 | OXTR |
| 4.5 | W | W | W | W | W | W | W | W | W | W |
| 4.46 | I | L | I | I | L | I | I | I | I | V |
| 4.43-4.39 | R(4.41) | R(4.41) | K(4.43) | R(4.41) | R(4.41) | R(4.41) | R(4.41) | K(4.43) | K(4.43) | R(4.43) |
| 2.41 | Y | Y | Y | Y | F | Y | Y | Y | Y | F |

### Example 8

### The Calculated Structure of the GPCR ADORA2A Based on the Empirically-Determined Structure for Human β₂AR

The structure of the GPCR ADORA2A (SEQ ID NO: 4) was calculated based on the empirically determined structure for 132AR in Appendix I. The methods used for calculating the structure are as in Example 7. The PDB file for the resulting calculated structure is shown in Appendix III. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 9

### The Calculated Structure of the GPCR ADRA1A Based on the Empirically-Determined Structure for Human β₂AR.

The structure of the GPCR ADRA1A (SEQ ID NO: 5) was calculated based on the empirically determined structure for β2AR in Appendix I. The methods used for calculating the structure are as in Example 7. The PDB file for the resulting calculated structure is shown in Appendix IV. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 10

### The Calculated Structure of the GPCR CHRM1 Based on the Empirically-Determined Structure for Human β₂AR

The structure of the GPCR CHRM1 (SEQ ID NO: 6) was calculated based on the empirically determined structure for β2AR in Appendix I. The methods used for calculating the structure are as in Example 7. The PDB file for the resulting calculated structure is shown in Appendix V. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 11

### The Calculated Structure of the GPCR DRD2 Based on the Empirically-Determined Structure for Human β₂AR

The structure of the GPCR DRD2 (SEQ ID NO: 7) was calculated based on the empirically determined structure for β2AR in Appendix I. The methods used for calculating the structure are as in Example 7. The PDB file for the resulting calculated structure is shown in Appendix VI. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 12

### The Calculated Structure of the GPCR EDG1 Based on the Empirically-Determined Structure for Human β₂AR

The structure of the GPCR EDG1 (SEQ ID NO: 8) was calculated based on the empirically determined structure for β2AR in Appendix I. The methods used for calculating the structure are as in Example 7. The PDB file for the resulting calculated structure is shown in Appendix VII. The residues important for ligand binding in the GPCR are shown in Table 5 using the Ballesteros-Weinstein numbering scheme described above. The residues important for binding to the CCM are shown in Table 6 using the Ballesteros-Weinstein numbering scheme described above.

### Example 13

### The Calculated Structure of the GPCR HTR1A Based on the Empirically-Determined Structure for Human β₂AR

The structure of the GPCR HTR1A (SEQ ID NO: 9) was calculated based on the empirically determined structure for β2AR in Appendix I. The methods used for calculating the structure are as in Example 7. The PDB file for the resulting calculated structure is shown in Appendix VIII. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 14

### The Calculated Structure of the GPCR MC2R Based on the Empirically-Determined Structure for Human β₂AR

The structure of the GPCR MC2R (SEQ ID NO: 10) was calculated based on the empirically determined structure for 132AR in Appendix I. The methods used for calculating the structure are as in Example 7. The PDB file for the resulting calculated structure is shown in Appendix IX. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 15

### The Calculated Structure of the GPCR NTSR1 Based on the Empirically-Determined Structure for Human β₂AR

The structure of the GPCR NTSR1 (SEQ ID NO: 11) was calculated based on the empirically determined structure for β2AR in Appendix I. The methods used for calculating the structure are as in Example 7. The PDB file for the resulting calculated structure is shown in Appendix X. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 16

### The Calculated Structure of the GPCR OXTR Based on the Empirically-Determined Structure for Human β₂AR

The structure of the GPCR OXTR (SEQ ID NO: 12) was calculated based on the empirically determined structure for β2AR in Appendix I. The methods used for calculating the structure are as in Example 7. The PDB file for the resulting calculated structure is shown in Appendix XI. The residues important for ligand binding to the GPCR are shown in Table 5 using the Ballesteros-Weinstein numbering scheme described above. The residues important for binding to the CCM are shown in Table 6 using the Ballesteros-Weinstein numbering scheme described above.

### Example 17

### Mutagenesis Studies

A series of in silico and in vitro mutagenesis studies was performed to determine the amino acid requirements of the CCM motif (Table 8.5). In silico mutagenesis coupled with calculation of enthalpy of interaction of cholesterol shows that most single mutations affect the calculated binding of cholesterol modestly. With the exception of the W158A and W158F mutants, all of the mutations retained the ability to bind cholesterol. These results were confirmed experimentally using a size exclusion chromatography assay sensitive to any stabilizing effect of cholesteryl hemisuccinate (“CHS”) on the protein. The construct used to generate the mutants (“wt” in Table 8.5) was a construct encoding β₂AR comprising the E122W mutation, as described herein.

To generate protein for the assay, baculovirus stocks were generated for each mutant and each used to infect a 250 mL culture of Spodoptera frugiperdia (Sf9) insect cells at a cell density of 2.00×10⁶ cells/mL. Protein was allowed to express for 48 hours following infection, at which time the Sf9 cells were collected by centrifugation and frozen at −80° C. The cultures were then processed individually as follows: The frozen cell paste was thawed and resuspended into 25 mL of lysis buffer containing 10 mM Hepes pH 7.5 10 mM MgCl₂ and 20 mM KCl. The β₂AR antagonist timolol was added to a concentration of 2 mM and allowed to bind for 30 minutes. The solution was then diluted two-fold with a 2× concentrated solubilization buffer to achieve a final concentration of 50 mM Hepes pH 7.5, 150 mM NaCl, 0.5% w/v Dodecyl maltoside (DDM) and 0.1% w/v cholesteryl hemisuccinate (CHS). The solubilization was allowed to incubate for two hours followed by a centrifugation at 70,000 rpm for 30 minutes in a Beckman Ti70 rotor. The resulting supernatant was collected and allowed to bind overnight to an α-FLAG sepharose resin (Sigma) which specifically binds the N-terminal FLAG epitope on each mutant. The next day the resin was washed with 10 column volumes (CV) of 50 mM Hepes pH 7.5, 300 mM NaCl, 0.05% w/v DDM, 0.01% w/v CHS and 1 mM timolol. The proteins were then eluted using 150 μg/mL of FLAG elution peptide (Sigma) and concentrated to 100 uL using a Vivaspin 100 kDa microconcentrator.

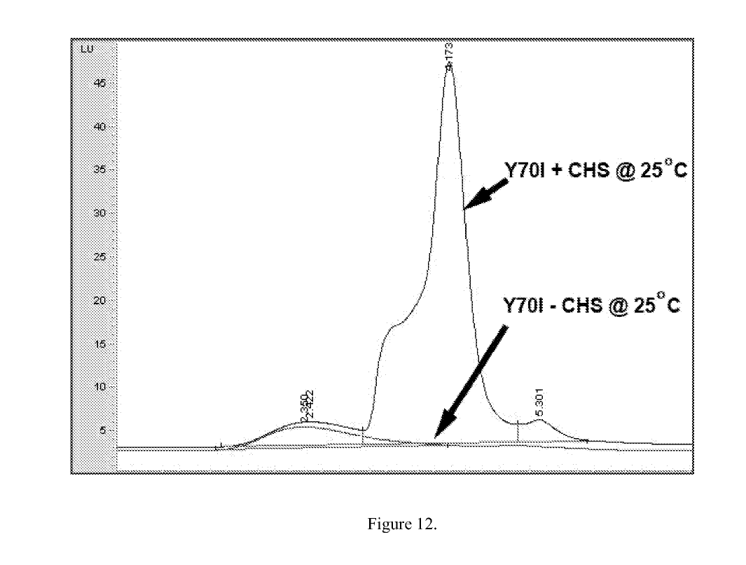

The ability of CHS to stabilize each purified β2AR mutant was then assessed by size exclusion chromatography (“SEC”) of the protein, comparing mobile phase without CHS (“−CHS”) to mobile phase with CHS (“+CHS”). Specifically, the SEC running buffer was warmed to 25 degrees and a 10 μL aliquot of purified protein containing the mutation of interest was injected. Denatured protein is retained by the SEC column's stationary phase indefinitely whereas folded protein elutes with a retention time of 4.2 minutes as shown. Protein elution is measured by tryptophan fluorescence using an excitation wavelength of 280 nM and an emission wavelength of 350 nM. An example of the results is shown in FIG. 12 for the Y701 mutant. The results for the analyzed mutants are compiled in Table 8.5. The results show a CHS-induced stabilization of the protein, i.e., where CHS is included in the running buffer β2AR is not denatured and elutes at a standard retention time, whereas when CHS is not included in the running buffer, the protein is retained on the column and is not seen.

This conclusion is supported by the experimental results.

TABLE 8.5
Mutagenesis of the CCM motif

| | relative | CHS | Modeled Cholesterol |
|---|---|---|---|
| | Expression | induced stability | binding (kcal) |
| WT | 1.00 | yes | −19 |
| Y70F | 1.30 | yes | −18.6 |
| Y70A | 0.28 | yes | −18.8 |
| Y70I | 1.13 | yes | −17.8 |
| R151V | 0.93 | yes | −17.5 |
| R151A | 1.74 | yes | −18.4 |
| R151K | 0.70 | yes | −18.8 |
| I154V | 0.70 | yes | −17.7 |
| I154L | 0.13 | yes | −17.9 |
| I154A | 0.48 | yes | −16.7 |
| I154S | 0.41 | yes | −15.4 |
| W158A | 0.47 | no | −15.6 |
| W158F | 0.33 | no | −17.2 |

The W158 mutations likely are further destabilized by loss of an important hydrogen bond with Ser74 on helix II, obscuring any stabilizing effect of cholesterol from detection according to this method. As noted above, all of the mutant proteins shown in Table 8.5 retained their ability to bind cholesterol.

Cholesterol binding by the CCM binding site is thus mainly due to steric complementarity, which is determined by the overall topology of the CCM site and delineated by the helical positions. Therefore, multiple simultaneous mutations will typically be necessary to achieve an appreciable loss of binding affinity. All 203 known human class A GPCRs are analzyed in a similar manner so that the complete set of residues that do not support cholesterol binding in this region are obtained.

### Example 18

### Small Molecule Docking Studies

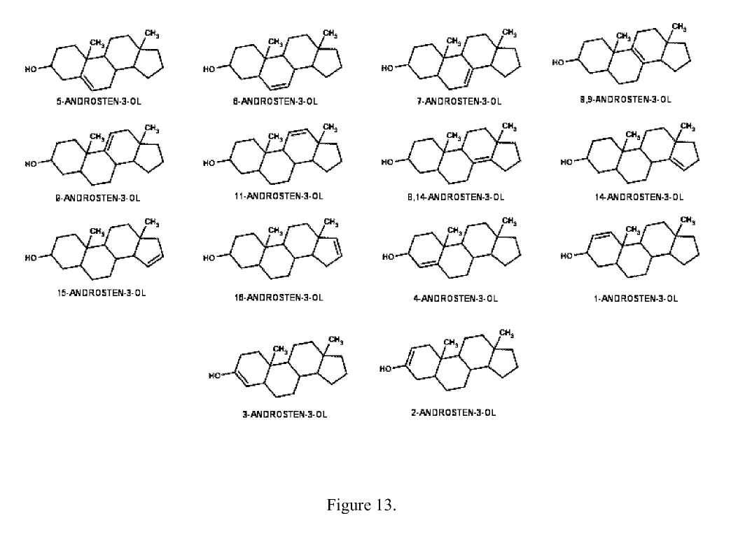

Based on the binding interactions, described herein, between β2AR and cholesterol in the crystal structure we hypothesize that steric complementarity between the sterol ring system and the protein is the largest contributor to the overall binding energy. This hypothesis is supported by the deletion of the hydrogen bond donor R151 with retention of cholesterol binding. Small molecule docking studies were therefore focused around androstenol based rather than cholesterol based sterol compounds (FIG. 13). Exploration of the docking requirements for binding of sterol rings in this area revealed that the docking algorithm, as implemented in ICM, can correctly place the molecule regardless of the presence of a single double bond at any position of the four ring sterol system. Likewise, the existence of two double bonds within the ring system should not significantly affect the ability of the sterol ring system to bind in this location. Planar aromatic species, however, will abrogate predicted binding interactions and thus should be avoided as replacements of any of the sterol rings in compound development.

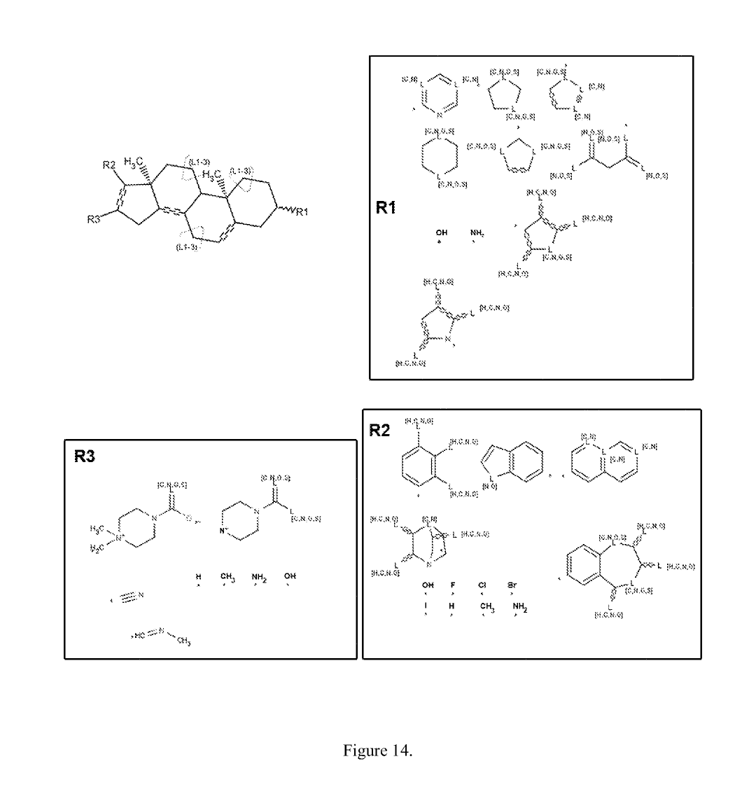

With the appropriate docking algorithm now implemented, the R group requirements are explored through Markusch enumeration and estimation of binding affinities, in addition to virtual ligand screening for new ring structures capable of binding in this or adjacent areas (FIG. 14).

While the invention has been particularly shown and described with reference to a preferred embodiment and several alternate embodiments, it will be understood by persons skilled in the relevant art that various changes in form and details can be made therein without departing from the spirit and scope of the invention.

It should be noted that the language used in the specification has been principally selected for readability and instructional purposes, and can not have been selected to delineate or circumscribe the inventive subject matter. Accordingly, the disclosure of the present invention is intended to be illustrative, but not limiting, of the scope of the invention, which is set forth in the claims.

All references, issued patents and patent applications cited within the body of the instant specification are hereby incorporated by reference in their entirety, for all purposes.

TABLE 9

| Name | Description | Author | Year |
|---|---|---|---|
| C-BOP | Coordinate-Based | E. Sandelin | 2005 |
| | Organization of Proteins | | |
| CAALIGN | Cα Align | T. J. Oldfield | 2007 |
| CBA | Consistency Based | J. Ebert | 2006 |
| | Alignment | | |
| CE/CE-MC | Combinatorial Extension -- | I. Shindyalov | 2000 |
| | Monte Carlo | | |
| CLEMAPS | Conformation-based | W-M. Zheng | 2007 |
| | alphabet alignments | | |
| CTSS | Protein Structure | T. Can | 2004 |
| | Alignment Using Local | | |
| | Geometrical Features | | |
| CURVE | NA | D. Zhi | 2006 |
| DaliLite | Distance Matrix | L. Holm | 1993 |
| | Alignment | | |
| DEJAVU | NA | GJ. Kleywegt | 1997 |
| EXPRESSO | Fast Multiple Structural | C. Notredame et | 2007 |
| | Alignment using T- | al. | |
| | Coffee and Sap | | |
| FAST | FAST Alignment and | J. Zhu | 2004 |
| | Search Tool | | |
| FATCAT | Flexible Structure | Y. Ye & A. Godzik | 2003 |
| | AlignmenT by Chaining | | |
| | Aligned Fragment Pairs | | |
| | Allowing Twists | | |
| FLASH | Fast aLignment | E. S. C. Shih & M-J Hwang | 2003 |
| | Algorithm for finding | | |
| | Structural Homology of | | |
| | proteins | | |
| FlexProt | Flexible Alignment of | M. Shatsky & H. Wolfson | 2002 |
| | Protein Structures | | |
| GANGSTA | Genetic Algorithm for | B. Kolbeck et al. | 2006 |
| | Nonsequential and | | |
| | Gapped STructural Alignment | | |
| KENOBI/K2 | NA | Z. Weng | 2000 |
| LGA | Local-Global Alignment | A. Zemla | 2003 |
| LOCK | Hierarchical protein | AP. Singh | 1997 |
| | structure superposition | | |
| LOCK 2 | Improvements over | J. Shapiro | 2003 |
| | LOCK | | |
| LOVOALIGN | Low Order Value | Andreani et al. | 2006 |
| | Optimization methods | | |
| | for Structural Alignment | | |
| MALECON | NA | S. Wodak | 2004 |
| MAMMOTH | MAtching Molecular | AR. Ortiz | 2002 |
| | Models Obtained from | | |
| | Theory | | |
| MAMMOTH- | MAMMOTH-based | D. Lupyan | 2005 |
| mult | multiple structure | | |
| | alignment | | |
| MASS | Multiple Alignment by | O. Dror & H. Wolfson | 2003 |
| | Secondary Structure | | |
| MatAlign | Protein Structure | Z. Aung & K. L. Tan | 2006 |
| | Comparison by Matrix | | |
| | Alignment | | |
| Matchprot | Comparison of protein | S. Bhattacharya | 2007 |
| | structures by growing | et al. | |
| | neighborhood alignments | | |
| Matras | MArkovian TRAnsition | K. Nishikawa | 2000 |
| | of protein Structure | | |
| Matt | Multiple Alignment with | M. Menke | 2008 |
| | Translations and Twists | | |
| MolCom | NA | S. D. O'Hearn | 2003 |
| MultiProt | Multiple Alignment of | M. Shatsky & H. Wolfson | 2004 |
| | Protein Structures | | |
| MUSTANG | MUltiple STructural | A. S. Konagurthu | 2005 |
| | AligNment AlGorithm | et al. | |
| POSA | Partial Order Structure | Y. Ye & A. Godzik | 2005 |
| | Alignment | | |
| PRIDE | PRobaility of IDEntity | S. Pongor | 2002 |
| PrISM | Protein Informatics | B. Honig | 2000 |
| | Systems for Modeling | | |
| ProFit | Protein least-squares | ACR. Martin | 1996 |
| | Fitting | | |
| Protein3Dfit | NA | D. Schomburg | 1994 |
| PyMOL | “super” command does | W. L. DeLano | 2007 |
| | sequence-independent | | |
| | 3D alignment | | |
| RAPIDO | Rapid Alignment of | R. Mosca & T. R. Schneider | 2008 |
| | Protein structures In the | | |
| | presence of Domain | | |
| | mOvements | | |
| SARF2 | Spatial ARrangements of | N. Alexandrov | 1996 |
| | Backbone Fragments | | |
| SCALI | Structural Core | C. Bystroff | 2004 |
| | ALIgnment of proteins | | |
| SHEBA | Structural Homology by | B. Lee | 2000 |
| | Environment-Based | | |
| | Alignment | | |
| SSAP | Sequential Structure | C. Orengo & W. Taylor | 1989 |
| | Alignment Program | | |
| SSGS | Secondary Structure | G. Wainreb et al. | 2006 |
| | Guided Superimposition | | |
| SSM | Secondary Structure | E. Krissinel | 2003 |
| | Matching | | |
| STAMP | STructural Alignment of | R. Russell & G. Barton | 1992 |
| | Multiple Proteins | | |
| STRAP | STRucture based | C. Gille | 2006 |
| | Alignment Program | | |
| TALI | Torsion Angle | X. Mioa | 2006 |
| | ALIgnment | | |
| TetraDA | Tetrahedral | J. Roach | 2005 |
| | Decomposition | | |
| | Alignment | | |
| TM-align | TM-score based protein | Y. Zhang & J. Skolnick | 2005 |
| | structure alignment | | |
| TopMatch | Protein structure | M. Sippl & M. Wiederstein | 2008 |
| | alignment and | | |
| | visualization of | | |
| | structural similarities | | |
| TOPOFIT | Alignment as a | VA. Ilyin | 2004 |
| | superimposition of | | |
| | common volumes at a | | |
| | topomax point | | |
| UCSF | see MatchMaker tool | E. Meng et al. | 2006 |
| Chimera | and “matchmaker” | | |
| | command | | |
| URMS | Unit-vector RMSD | K. Kedem | 2003 |
| VAST | Vector Alignment | S. Bryant | 1996 |
| | Search Tool | | |
| Vorolign | Fast structure alignment | F. Birzele et al. | 2007 |
| | using Voronoi contacts | | |
| YAKUSA | Internal Co-ordinates | M. Carpentier et | 2005 |
| | and BLAST type | al. algorithm | |

### REFERENCES CITED

Alexandrov, A. I., Mileni, M., Chien, E. Y. T., Hanson, M. A., and Stevens, R. C. (2008). Microscale Fluorescent Thermal Stability Assay for Membrane Proteins. Structure 16, 351-359.
Baker, J. G., Hall, I. P., and Hill, S. J. (2003). Agonist and inverse agonist actions of beta-blockers at the human beta 2-adrenoceptor provide evidence for agonist-directed signaling. Mol. Pharmacol. 64, 1357-1369.
Ballesteros, J. A., and Weinstein, H. (1995). Integrated methods for the construction of three dimensional models and computational probing of structure-function relations in G-protein coupled receptors. Methods Neurosci. 25, 366-428.
Burger, K., Gimpl, G., and Fahrenholz, F. (2000). Regulation of receptor function by cholesterol. Cell. Mol. Life. Sci. 57, 1577-1592.
Cherezov, V., and Caffrey, M. (2003). Nano-volume plates with excellent optical properties for fast, inexpensive crystallization screening of membrane proteins. J. Appl. Cryst. 36: 1372-1377.
Cherezov, V., Peddi, A., Muthusubramaniam, L., Zheng, Y. F., and Caffrey, M. (2004). A robotic system for crystallizing membrane and soluble proteins in lipidic mesophases. Acta Crystallogr D Biol Crystallogr 60, 1795-1807.
Cherezov, V., Rosenbaum, D. M., Hanson, M. A., Rasmussen, S. G., Thian, F. S., Kobilka, T. S., Choi, H. J., Kuhn, P., Weis, W. I., Kobilka, B. K., and Stevens, R. C. (2007). High-resolution crystal structure of an engineered human beta2-adrenergic G protein-coupled receptor. Science (New York, N.Y. 318, 1258-1265.
Chidiac, P., Hebert, T. E., Valiquette, M., Dennis, M., and Bouvier, M. (1994). Inverse agonist activity of beta-adrenergic antagonists. Mol. Pharmacol. 45, 490-499.
DeDecker, B. S., O'Brien, R., Fleming, P. J., Geiger, J. H., Jackson, S. P., and Sigler, P. B. (1996). The crystal structure of a hyperthermophilic archaeal TATA-box binding protein. J. Mol. Biol. 264, 1072-1084.
Eilers, M., Patel, A. B., Liu, W., and Smith, S. O. (2002). Comparison of helix interactions in membrane and soluble alpha-bundle proteins. Biophys. J. 82, 2720-2736.
Eilers, M., Shekar, S. C., Shieh, T., Smith, S. O., and Fleming, P. J. (2000). Internal packing of helical membrane proteins. Proc. Natl. Acad. Sci. U.S.A. 97, 5796-5801.
Epand, R. F., Thomas, A., Brasseur, R., Vishwanathan, S. A., Hunter, E., and Epand, R. M. (2006). Juxtamembrane protein segments that contribute to recruitment of cholesterol into domains. Biochemistry 45, 6105-6114.
Epand, R. M., Sayer, B. G., and Epand, R. F. (2005). Caveolin scaffolding region and cholesterol-rich domains in membranes. J. Mol. Biol. 345, 339-350.
Gimpl, G., Burger, K., and Fahrenholz, F. (1997). Cholesterol as modulator of receptor function. Biochemistry 36, 10959-10974.
Gimpl, G., and Fahrenholz, F. (2002). Cholesterol as stabilizer of the oxytocin receptor. Biochim. Biophys. Acta 1564, 384-392.
Green, S A, Spasoff A P, Coleman R A, Johnson M, and Liggett S B (1996). Sustained activation of a G protein-coupled receptor via “anchored” agonist binding. JBC. 271, 24029-24035.
Grossfield, A., Feller, S. E., and Pitman, M. C. (2006). A role for direct interactions in the modulation of rhodopsin by omega-3 polyunsaturated lipids. Proc. Natl. Acad. Sci. U.S.A. 103, 4888-4893.
Hannawacker, A., Krasel, C., and Lohse, M. J. (2002). Mutation of Asn293 to Asp in transmembrane helix VI abolishes agonist-induced but not constitutive activity of the beta(2)-adrenergic receptor. Mol. Pharmacol. 62, 1431-1437.
Jamin, N., Neumann, J. M., Ostuni, M. A., Vu, T. K., Yao, Z. X., Murail, S., Robert, J. C., Giatzakis, C., Papadopoulos, V., and Lacapere, J. J. (2005). Characterization of the cholesterol recognition amino acid consensus sequence of the peripheral-type benzodiazepine receptor. Mol. Endocrinol. 19, 588-594.
Lee, A. G. (2004). How lipids affect the activities of integral membrane proteins. Biochim. Biophys. Acta 1666, 62-87.
Li, H., and Papadopoulos, V. (1998). Peripheral-type benzodiazepine receptor function in cholesterol transport. Identification of a putative cholesterol recognition/interaction amino acid sequence and consensus pattern. Endocrinology 139, 4991-4997.
Marheineke, K., Grunewald, S., Christie, W., and Reilander, H. (1998). Lipid composition of Spodoptera frugiperda (Sf9) and Trichoplusia ni (Tn) insect cells used for baculovirus infection. FEBS letters 441, 49-52.
Milligan, G., Svoboda, P., and Brown, C. M. (1994). Why are there so many adrenoceptor subtypes? Biochem. Pharmacol. 48, 1059-1071.
Niu, S. L., Mitchell, D. C., and Litman, B. J. (2002). Manipulation of cholesterol levels in rod disk membranes by methyl-beta-cyclodextrin: effects on receptor activation. J. Biol. Chem. 277, 20139-20145.
Ostrom, R. S., Gregorian, C., Drenan, R. M., Xiang, Y., Regan, J. W., and Insel, P. A. (2001). Receptor number and caveolar co-localization determine receptor coupling efficiency to adenylyl cyclase. J. Biol. Chem. 276, 42063-42069.
Pang, L., Graziano, M., and Wang, S. (1999). Membrane cholesterol modulates galanin-GalR2 interaction. Biochemistry 38, 12003-12011.
Pattabiraman, N., Ward, K. B., and Fleming, P. J. (1995). Occluded molecular surface: analysis of protein packing J. Mol. Recognit. 8, 334-344.
Pucadyil, T. J., and Chattopadhyay, A. (2004). Cholesterol modulates ligand binding and G-protein coupling to serotonin1A receptors from bovine hippocampus. Biochim. Biophys. Acta 1663, 188-200.
Pucadyil, T. J., and Chattopadhyay, A. (2006). Role of cholesterol in the function and organization of G-protein coupled receptors. Prog. Lipid Res. 45, 295-333.
Rasmussen, S. G., Choi, H. J., Rosenbaum, D. M., Kobilka, T. S., Thian, F. S., Edwards, P. C., Burghammer, M., Ratnala, V. R., Sanishvili, R., Fischetti, R. F., et al. (2007). Crystal structure of the human beta2 adrenergic G-protein-coupled receptor. Nature 450, 383-387.
Rosenbaum, D. M., Cherezov, V., Hanson, M. A., Rasmussen, S. G., Thian, F. S., Kobilka, T. S., Choi, H. J., Yao, X. J., Weis, W. I., Stevens, R. C., and Kobilka, B. K. (2007). GPCR engineering yields high-resolution structural insights into beta2-adrenergic receptor function. Science (New York, N.Y. 318, 1266-1273.
Roth, C. B., Hanson, M. A., and Stevens, R. C. (2008). Stabilization of the human beta2-adrenergic receptor TM4-TM3-TM5 helix interface by mutagenesis of Glu122(3.41), a critical residue in GPCR structure. J. Mol. Biol. 376, 1305-1319.
Rybin, V. O., Xu, X., Lisanti, M. P., and Steinberg, S. F. (2000). Differential Targeting of beta-Adrenergic Receptor Subtypes and Adenylyl Cyclase to Cardiomyocyte Caveolae. A MECHANISM TO FUNCTIONALLY REGULATE THE cAMP SIGNALING PATHWAY. J. Biol. Chem. 275, 41447-41457.
Simons, K., and Ikonen, E. (2000). How cells handle cholesterol. Science (New York, N.Y. 290, 1721-1726.
Steinberg, S. F. (2004). [beta]2-Adrenergic receptor signaling complexes in cardiomyocyte caveolae/lipid rafts. J. Mol. Cell. Cardiol. 37, 407-415.
Takeda, S., Kadowaki, S., Haga, T., Takaesu, H., and Mitaku, S. (2002). Identification of G protein-coupled receptor genes from the human genome sequence. FEBS Lett. 520, 97-101.
Wieland, K., Zuurmond, H. M., Krasel, C., Ijzerman, A. P., and Lohse, M. J. (1996). Involvement of Asn-293 in stereospecific agonist recognition and in activation of the beta 2-adrenergic receptor. Proc. Natl. Acad. Sci. U.S.A. 93, 9276-9281.
Xiang, Y., and Kobilka, B. (2003). The PDZ-binding motif of the beta2-adrenoceptor is essential for physiologic signaling and trafficking in cardiac myocytes. Proc. Natl. Acad. Sci. U.S.A. 100, 10776-10781.
Yao, Z., and Kobilka, B. (2005). Using synthetic lipids to stabilize purified beta2 adrenoceptor in detergent micelles. Anal. Biochem. 343, 344-346.
Zimmerman, T. J., and Kaufman, H. E. (1977). Timolol. A beta-adrenergic blocking agent for the treatment of glaucoma. Arch. Ophthalmol. 95, 601-604.

### Informal Sequence Listing

| SEQ ID NO | DESCRIPTION | SEQUENCE |
|---|---|---|
| SEQ ID NO: 1 | Human β2 | MGQPGNGSAFLLAPNRSHAPDHDVTQQRDEVWVVGMGIVMSLIVLAIVFG |
| | adrenergic receptor; | NVLVITAIAKFERLQTVTNYFITSLACADLVMGLAVVPFGAAHILMKMWT |
| | protein sequence | FGNFWCEFWTSIDVLCVTASIETLCVIAVDRYFAITSPFKYQSLLTKNKA |
| | | RVIILMVWIVSGLTSFLPIQMHWYRATHQEAINCYANETCCDFFTNQAYA |
| | | IASSIVSFYVPLVIMVFVYSRVFQEAKRQLQKIDKSEGRFHVQNLSQVEQ |
| | | DGRTGHGLRRSSKFCLKEHKALKTLGIIMGTFTLCWLPFFIVNIVHVIQD |
| | | NLIRKEVYILLNWIGYVNSGFNPLIYCRSPDFRIAFQELLCLRRSSLKAY |
| | | GNGYSSNGNTGEQSGYHVEQEKENKLLCEDLPGTEDFVGHQGTVPSDNID |
| | | SQGRNCSTNDSLL |
| SEQ ID NO: 2 | Human β2 | gcacataacgggcagaacgcactgcgaagcggcttcttcagagcacgggc |
| | adrenergic receptor; | tggaactggcaggcaccgcgagcccctagcacccgacaagctgagtgtgc |
| | nucleotide sequence | aggacgagtccccaccacacccacaccacagccgctgaatgaggcttcca |
| | | ggcgtccgctcgcggcccgcagagccccgccgtgggtccgcccgctgagg |
| | | cgcccccagccagtgcgctcacctgccagactgcgcgccatggggcaacc |
| | | cgggaacggcagcgccttcttgctggcacccaatagaagccatgcgccgg |
| | | accacgacgtcacgcagcaaagggacgaggtgtgggtggtgggcatgggc |
| | | atcgtcatgtctctcatcgtcctggccatcgtgtttggcaatgtgctggt |
| | | catcacagccattgccaagttcgagcgtctgcagacggtcaccaactact |
| | | tcatcacttcactggcctgtgctgatctggtcatgggcctggcagtggtg |
| | | ccctttggggccgcccatattcttatgaaaatgtggacttttggcaactt |
| | | ctggtgcgagttttggacttccattgatgtgctgtgcgtcacggccagca |
| | | ttgagaccctgtgcgtgatcgcagtggatcgctactttgccattacttca |
| | | cctttcaagtaccagagcctgctgaccaagaataaggccagggtgatcat |
| | | tctgatggtgtggattgtgtcaggccttacctccttcttgcccattcaga |
| | | tgcactggtaccgggccacccaccaggaagccatcaactgctatgccaat |
| | | gagacctgctgtgacttcttcacgaaccaagcctatgccattgcctcttc |
| | | catcgtgtccttctacgttcccctggtgatcatggtcttcgtctactcca |
| | | gggtctttcaggaggccaaaaggcagctccagaagattgacaaatctgag |
| | | ggccgcttccatgtccagaaccttagccaggtggagcaggatgggcggac |
| | | ggggcatggactccgcagatcttccaagttctgcttgaaggagcacaaag |
| | | ccctcaagacgttaggcatcatcatgggcactttcaccctctgctggctg |
| | | cccttcttcatcgttaacattgtgcatgtgatccaggataacctcatccg |
| | | taaggaagtttacatcctcctaaattggataggctatgtcaattctggtt |
| | | tcaatccccttatctactgccggagcccagatttcaggattgccttccag |
| | | gagcttctgtgcctgcgcaggtcttctttgaaggcctatgggaatggcta |
| | | ctccagcaacggcaacacaggggagcagagtggatatcacgtggaacagg |
| | | agaaagaaaataaactgctgtgtgaagacctcccaggcacggaagacttt |
| | | gtgggccatcaaggtactgtgcctagcgataacattgattcacaagggag |
| | | gaattgtagtacaaatgactcactgctgtaaagcagtttttctactttta |
| | | aagacccccccccccaacagaacactaaacagactatttaacttgagggt |
| | | aataaacttagaataaaattgtaaaattgtatagagatatgcagaaggaa |
| | | gggcatccttctgccttttttatttttttaagctgtaaaaagagagaaaa |
| | | cttatttgagtgattatttgttatttgtacagttcagttcctctttgcat |
| | | ggaatttgtaagtttatgtctaaagagctttagtcctagaggacctgagt |
| | | ctgctatattttcatgacttttccatgtatctacctcactattcaagtat |
| | | taggggtaatatattgctgctggtaatttgtatctgaaggagattttcct |
| | | tcctacacccttggacttgaggattttgagtatctcggacctttcagctg |
| | | tgaacatggactcttcccccactcctcttatttgctcacacggggtattt |
| | | taggcagggatttgaggagcagcttcagttgttttcccgagcaaagtcta |
| | | aagtttacagtaaataaattgtttgaccatgcc |
| SEQ ID NO: 3 | TACR1; tachykinin | MDNVLPVDSDLSPNISTNTSEPNQFVQPAWQIVLWAAAYTVIVVTSVVGN |
| | receptor 1; | VVVMWIILAHKRMRTVTNYFLVNLAFAEASMAAFNTVVNFTYAVHNEWYY |
| | NM_001058; | GLFYCKFHNFFPIAAVFASIYSMTAVAFDRYMAIIHPLQPRLSATATKVV |
| | protein sequence | ICVIWVLALLLAFPQGYYSTTETMPSRVVCMIEWPEHPNKIYEKVYHICV |
| | | TVLIYFLPLLVIGYAYTVVGITLWASEIPGDSSDRYHEQVSAKRKVVKMM |
| | | IVVVCTFAICWLPFHIFFLLPYINPDLYLKKFIQQVYLAIMWLAMSSTMY |
| | | NPIIYCCLNDRFRLGFKHAFRCCPFISAGDYEGLEMKSTRYLQTQGSVYK |
| | | VSRLETTISTVVGAHEEEPEDGPKATPSSLDLTSNCSSRSDSKTMTESFS |
| | | FSSNVLS |
| SEQ ID NO: 4 | ADORA2A; | MPIMGSSVYITVELAIAVLAILGNVLVCWAVWLNSNLQNVTNYFVVSLAA |
| | adenosine A2a | ADIAVGVLAIPFAITISTGFCAACHGCLFIACFVLVLTQSSIFSLLAIAI |
| | receptor; | DRYIAIRIPLRYNGLVTGTRAKGIIAICWVLSFAIGLTPMLGWNNCGQPK |
| | NM_000675; | EGKNHSQGCGEGQVACLFEDVVPMNYMVYFNFFACVLVPLLLMLGVYLRI |
| | protein sequence | FLAARRQLKQMESQPLPGERARSTLQKEVHAAKSLAIIVGLFALCWLPLH |
| | | IINCFTFFCPDCSHAPLWLMYLAIVLSHTNSVVNPFIYAYRIREFRQTFR |
| | | KIIRSHVLRQQEPFKAAGTSARVLAAHGSDGEQVSLRLNGHPPGVWANGS |
| | | APHPERRPNGYALGLVSGGSAQESQGNTGLPDVELLSHELKGVCPEPPGL |
| | | DDPLAQDGAGVS |
| SEQ ID NO: 5 | ADRA1A; | MVFLSGNASDSSNCTQPPAPVNISKAILLGVILGGLILFGVLGNILVILS |
| | adrenergic, alpha- | VACHRHLHSVTHYYIVNLAVADLLLTSTVLPFSAIFEVLGYWAFGRVFCN |
| | 1A-, receptor; | IWAAVDVLCCTASIMGLCIISIDRYIGVSYPLRYPTIVTQRRGLMALLCV |
| | NM_000680; | WALSLVISIGPLFGWRQPAPEDETICQINEEPGYVLFSALGSFYLPLAII |
| | protein sequence | LVMYCRVYVVAKRESRGLKSGLKTDKSDSEQVTLRIHRKNAPAGGSGMAS |
| | | AKTKTHFSVRLLKFSREKKAAKTLGIVVGCFVLCWLPFFLVMPIGSFFPD |
| | | FKPSETVFKIVFWLGYLNSCINPIIYPCSSQEFKKAFQNVLRIQCLCRKQ |
| | | SSKHALGYTLHPPSQAVEGQHKDMVRIPVGSRETFYRISKTDGVCEWKFF |
| | | SSMPRGSARITVSKDQSSCTTARVRSKSFLQVCCCVGPSTPSLDKNHQVP |
| | | TIKVHTISLSENGEEV |
| SEQ ID NO: 6 | CHRM1; | MNTSAPPAVSPNITVLAPGKGPWQVAFIGITTGLLSLATVTGNLLVLISF |
| | cholinergic | KVNTELKTVNNYFLLSLACADLIIGTFSMNLYTTYLLMGHWALGTLACDL |
| | receptor, | WLALDYVASNASVMNLLLISFDRYFSVTRPLSYRAKRTPRRAALMIGLAW |
| | muscarinic 1; | LVSFVLWAPAILFWQYLVGERTVLAGQCYIQFLSQPIITFGTAMAAFYLP |
| | NM_000738; | VTVMCTLYWRIYRETENRARELAALQGSETPGKGGGSSSSSERSQPGAEG |
| | protein sequence | SPETPPGRCCRCCRAPRLLQAYSWKEEEEEDEGSMESLTSSEGEEPGSEV |
| | | VIKMPMVDPEAQAPTKQPPRSSPNTVKRPTKKGRDRAGKGQKPRGKEQLA |
| | | KRKTFSLVKEKKAARTLSAILLAFILTWTPYNIMVLVSTFCKDCVPETLW |
| | | ELGYWLCYVNSTINPMCYALCNKAFRDTFRLLLLCRWDKRRWRKIPKRPG |
| | | SVHRTPSRQC |
| SEQ ID NO: 7 | DRD2; dopamine | MDPLNLSWYDDDLERQNWSRPFNGSDGKADRPHYNYYATLLTLLIAVIVF |
| | receptor D2; | GNVLVCMAVSREKALQTTTNYLIVSLAVADLLVATLVMPWVVYLEVVGEW |
| | NM_000795; | KFSRIHCDIFVTLDVMMCTASILNLCAISIDRYTAVAMPMLYNTRYSSKR |
| | protein sequence | RVTVMISIVWVLSFTISCPLLFGLNNADQNECIIANPAFVVYSSIVSFYV |
| | | PFIVTLLVYIKIYIVLRRRRKRVNTKRSSRAFRAHLRAPLKGNCTHPEDM |
| | | KLCTVIMKSNGSFPVNRRRVEAARRAQELEMEMLSSTSPPERTRYSPIPP |
| | | SHHQLTLPDPSHHGLHSTPDSPAKPEKNGHAKDHPKIAKIFEIQTMPNGK |
| | | TRTSLKTMSRRKLSQQKEKKATQMLAIVLGVFIICWLPFFITHILNIHCD |
| | | CNIPPVLYSAFTWLGYVNSAVNPIIYTTFNIEFRKAFLKILHC |
| SEQ ID NO: 8 | EDG1; endothelial | MGPTSVPLVKAHRSSVSDYVNYDIIVRHYNYTGKLNISADKENSIKLTSV |
| | differentiation, | VFILICCFIILENIFVLLTIWKTKKFHRPMYYFIGNLALSDLLAGVAYTA |
| | sphingolipidG- | NLLLSGATTYKLTPAQWFLREGSMFVALSASVFSLLAIAIERYITMLKMK |
| | protein-coupled | LHNGSNNFRLFLLISACWVISLILGGLPIMGWNCISALSSCSTVLPLYHK |
| | receptor, 1; | HYILFCTTVFTLLLLSIVILYCRIYSLVRTRSRRLTFRKNISKASRSSEK |
| | NM_001400; | SLALLKTVIIVLSVFIACWAPLFILLLLDVGCKVKTCDILFRAEYFLVLA |
| | protein sequence | VLNSGTNPIIYTLTNKEMRRAFIRIMSCCKCPSGDSAGKFKRPIIAGMEF |
| | | SRSKSDNSSHPQKDEGDNPETIMSSGNVNSSS |
| SEQ ID NO: 9 | HTR1A; 5- | MDVLSPGQGNNTTSPPAPFETGGNTTGISDVTVSYQVITSLLLGTLIFCA |
| | hydroxytryptamine | VLGNACVVAAIALERSLQNVANYLIGSLAVTDLMVSVLVLPMAALYQVLN |
| | (serotonin) receptor | KWTLGQVTCDLFIALDVLCCTSSILHLCAIALDRYWAITDPIDYVNKRTP |
| | 1A; NM_000524; | RRAAALISLTWLIGFLISIPPMLGWRTPEDRSDPDACTISKDHGYTIYST |
| | protein sequence | FGAFYIPLLLMLVLYGRIFRAARFRIRKTVKKVEKTGADTRHGASPAPQP |
| | | KKSVNGESGSRNWRLGVESKAGGALCANGAVRQGDDGAALEVIEVHRVGN |
| | | SKEHLPLPSEAGPTPCAPASFERKNERNAEAKRKMALARERKTVKTLGII |
| | | MGTFILCWLPFFIVALVLPFCESSCHMPTLLGAIINWLGYSNSLLNPVIY |
| | | AYFNKDFQNAFKKIIKCKFCRQ |
| SEQ ID NO: 10 | MC2R; | MKHIINSYENINNTARNNSDCPRVVLPEEIFFTISIVGVLENLIVLLAVF |
| | melanocortin 2 | KNKNLQAPMYFFICSLAISDMLGSLYKILENILIILRNMGYLKPRGSFET |
| | receptor | TADDIIDSLFVLSLLGSIFSLSVIAADRYITIFHALRYHSIVTMRRTVVV |
| | (adrenocorticotropic | LTVIWTFCTGTGITMVIFSHHVPTVITFTSLFPLMLVFILCLYVHMFLLA |
| | hormone); | RSHTRKISTLPRANMKGAITLTILLGVFIFCWAPFVLHVLLMTFCPSNPY |
| | NM_000529; | CACYMSLFQVNGMLIMCNAVIDPFIYAFRSPELRDAFKKMIFCSRYW |
| | protein sequence | |
| SEQ ID NO: 11 | NTSR1; | MRLNSSAPGTPGTPAADPFQRAQAGLEEALLAPGFGNASGNASERVLAAP |
| | neurotensin | SSELDVNTDIYSKVLVTAVYLALFVVGTVGNTVTAFTLARKKSLQSLQST |
| | receptor 1; | VHYHLGSLALSDLLTLLLAMPVELYNFIWVHHPWAFGDAGCRGYYFLRDA |
| | NM_002531; | CTYATALNVASLSVERYLAICHPFKAKTLMSRSRTKKFISAIWLASALLA |
| | protein sequence | VPMLFTMGEQNRSADGQHAGGLVCTPTIHTATVKVVIQVNTFMSFIFPMV |
| | | VISVLNTIIANKLTVMVRQAAEQGQVCTVGGEHSTFSMAIEPGRVQALRH |
| | | GVRVLRAVVIAFVVCWLPYHVRRLMFCYISDEQWTPFLYDFYHYFYMVTN |
| | | ALFYVSSTINPILYNLVSANFRHIFLATLACLCPVWRRRRKRPAFSRKAD |
| | | SVSSNHTLSSNATRETLY |
| SEQ ID NO: 12 | OXTR; oxytocin | MEGALAANWSAEAANASAAPPGAEGNRTAGPPRRNEALARVEVAVLCLIL |
| | receptor; | LLALSGNACVLLALRTTRQKHSRLFFFMKHLSIADLVVAVFQVLPQLLWD |
| | NM_000916; | ITFRFYGPDLLCRLVKYLQVVGMFASTYLLLLMSLDRCLAICQPLRSLRR |
| | protein sequence | RTDRLAVLATWLGCLVASAPQVHIFSLREVADGVFDCWAVFIQPWGPKAY |
| | | ITWITLAVYIVPVIVLAACYGLISFKIWQNLRLKTAAAAAAEAPEGAAAG |
| | | DGGRVALARVSSVKLISKAKIRTVKMTFIIVLAFIVCWTPFFFVQMWSVW |
| | | DANAPKEASAFIIVMLLASLNSCCNPWIYMLFTGHLFHELVQRFLCCSAS |
| | | YLKGRRLGETSASKKSNSSSFVLSHRSSSQRSCSQPSTA |

1. A method of identifying a compound that binds to a cholesterol consensus motif (CCM) of a G protein coupled receptor (GPCR) membrane protein by comparing a set of three-dimensional structures representing a set of candidate compounds with a three-dimensional molecular model of said CCM, comprising:
receiving said three-dimensional model of said CCM, wherein said three-dimensional model of said CCM comprises atomic co-ordinates of three or more residues selected from the set consisting of Ballesteros-Weinstein indexed residues [4.39-4.43(R,K)]---[4.50(W,Y)]---[4.46(I,V,L)]---[2.41(F,Y)];
receiving a set of compound three-dimensional models representing said set of candidate compounds, wherein each three-dimensional model comprises atomic co-ordinates of a candidate compound of the set of candidate compounds;
determining, for each of the set of compound three-dimensional models, a plurality of distance values indicating distances between said atomic co-ordinates of said candidate compound of the set of candidate compounds and said atomic coordinates of said three or more residues;
determining, for each of the set of compound three-dimensional models, a binding strength value based on the plurality of distance values determined for the compound three-dimensional model, wherein the binding strength value indicates the stability of a complex formed by said GPCR membrane protein and a compound represented by the compound three-dimensional model; and
storing a set of results indicating whether each candidate compound binds to the three-dimensional model based on the binding strength values.

2. The method of claim 1, wherein said GPCR membrane protein is selected from the group consisting of a class A GPCR, a class B GPCR, a class C GPCR, a class D GPCR, a class E GPCR, and a class F GPCR.

3. The method of claim 1, wherein said set comprises one or more members.

4. The method of claim 1, further comprising generating said three-dimensional molecular model of said cholesterol consensus motif (CCM).

5. The method of claim 4, wherein generating said three-dimensional molecular model of said cholesterol consensus motif (CCM) comprises:
identifying an amino acid sequence of said G protein coupled receptor (GPCR) membrane protein;
identifying said three or more residues of said amino acid sequence from said set consisting of Ballesteros-Weinstein indexed residues [4.39-4.43(R,K)]---[4.50(W,Y)]---[4.46(I,V,L)]---[2.41(F,Y)];
generating a three-dimensional model of said G protein coupled receptor (GPCR) membrane protein, said three-dimensional model of said G protein coupled receptor (GPCR) comprising atomic co-ordinates of residues in said amino acid sequence; and
generating said three-dimensional molecular model of said cholesterol consensus motif (CCM) responsive to selecting said atomic co-ordinates of said three or more residues based on said generated three-dimensional model of said G protein coupled receptor (GPCR) membrane protein.

6. The method of claim 5, further comprising generating said three-dimensional model of said G protein coupled receptor (GPCR) membrane protein using x-ray crystallography, electron crystallography, nuclear magnetic resonance, ab initio modeling, or a combination thereof.

7. The method of claim 5, further comprising generating said three-dimensional model of said G protein coupled receptor (GPCR) membrane protein using computational protein structure modeling.

8. The method of claim 1, further comprising:
receiving a three-dimensional model of a ligand binding site on said GPCR membrane protein, wherein said three-dimensional model of said ligand binding site comprises atomic co-ordinates for a plurality of ligand-binding residues selected from a second set of Ballesteros-Weinstein indexed residues;
determining, for each of the set of compound three-dimensional models, a plurality of distance values indicating distances between said atomic co-ordinates of said candidate compound of the set of candidate compounds and said atomic coordinates of said ligand-binding residues comprising said ligand binding site;
determining, for each of the set of compound three-dimensional models, a second binding strength value based on the plurality of distance values determined for the compound three-dimensional model, wherein the second binding strength value indicates the stability of a complex formed by said GPCR membrane protein and a compound represented by the compound three-dimensional model; and
storing a set of results indicating whether each candidate compound binds to the three-dimensional model based on the binding strength and second binding strength values.

9. The method of claim 8, wherein said GPCR membrane protein is β₂AR, and said second set of Ballesteros-Weinstein indexed residues are [3.32(D)]---[5.42(S)]---[5.43(S)]---[5.46(S)]---[6.44(F)]---[6.51(F)]---[6.52(F)]---[7.43(Y)].

10-23. (canceled)

24. The method of claim 1, wherein said set of candidate compounds comprises one or more candidate compounds selected from the group consisting of: 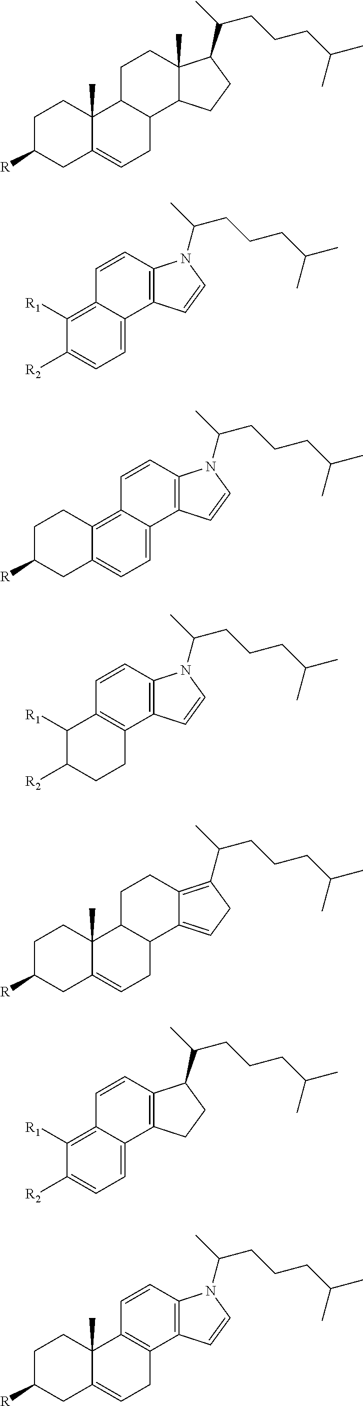 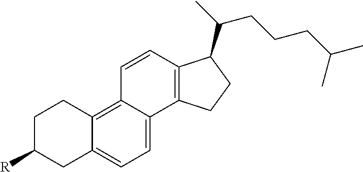
wherein R, R1, and R2 are independently selected from the group consisting of: hydrogen, acetate, aldehyde, benzoate, caproate, carboxylate, chloro, cyano, dichloroacetate, ethoxycarbonyl, ethyl ester, ethyleneketal, formate, hemisuccinate, hydrazone, oxime, phenylpropionate, proprionate, and sulphate.

25. The method of claim 1 or claim 8, further comprising the step of contacting said GPCR membrane protein with a molecule comprising an identified candidate compound.

26. The method of claim 25, wherein said molecule further comprises a moiety capable of competitively displacing a ligand from said GPCR membrane protein, wherein said ligand does not bind to said CCM.

27. The method of claim 26, wherein said ligand is selected from the group consisting of: timolol, isoproterenol, alprenolol, carazolol, and a ligand shown in Table 6.

28. The method of claim 25, further comprising characterizing a binding interaction between said GPCR membrane protein and said molecule comprising said identified candidate compound, and storing a result of said characterizing.

29. The method of claim 26, further comprising characterizing a binding interaction between said GPCR membrane protein and said molecule comprising said identified candidate compound, and storing a result of said characterizing.

30. The method of claim 28, wherein said characterization comprises determining an activation of a function of said GPCR membrane protein, an inhibition of a function of said GPCR membrane protein, an increase in expression of said GPCR membrane protein, a decrease in expression of said GPCR membrane protein, a displacement of a sterol bound to said CCM, or a stability measure for said GPCR membrane protein.

31. The method of claim 2, wherein said GPCR membrane protein is a class A GPCR membrane protein.

32. The method of claim 31, wherein said class A GPCR membrane protein is β₂AR.

33.-48. (canceled)

49. A crystalline form of β₂AR(E122W)-T4L having unit cell dimensions of a=40.0 Angstroms, b=75.7 Angstroms, and c=172.7 Angstroms.

50. The crystalline form of claim 49, wherein said space group of said crystalline form is P2₁2₁2₁.

51. The crystalline form of claim 49, wherein said crystalline form diffracts X-rays to resolution of 2.8 Angstroms.

52. A method of identifying a compound that binds to a ligand binding site of a G protein coupled receptor (GPCR) membrane protein by comparing a set of three-dimensional structures representing a set of candidate compounds with a three-dimensional molecular model of said ligand binding site, comprising:
receiving a three-dimensional model of a ligand binding site on said GPCR membrane protein, wherein said three-dimensional model of said ligand binding site comprises atomic co-ordinates for a plurality of ligand-binding residues selected from a set of Ballesteros-Weinstein indexed residues;
determining, for each of the set of compound three-dimensional models, a plurality of distance values indicating distances between said atomic co-ordinates of said candidate compound of the set of candidate compounds and said atomic coordinates of said ligand-binding residues comprising said ligand binding site;
determining, for each of the set of compound three-dimensional models, a binding strength value based on the plurality of distance values determined for the compound three-dimensional model, wherein the binding strength value indicates the stability of a complex formed by said GPCR membrane protein and a compound represented by the compound three-dimensional model; and
storing a set of results indicating whether each candidate compound binds to the three-dimensional model based on the binding strength values.

53.-79. (canceled)
